(19) United States
(12) Patent Application Publication
(10) Pub. No.: US 2004/0009478 A1
(43) Pub. Date: Jan. 15, 2004

(54) Human nucleic acid sequences from prostate tumor tissue

(75) Inventors: Thomas Specht, Berlin (DE); Bernd Hinzmann, Berlin (DE); Armin Schmitt, Berlin (DE); Christian Pilarsky, Schonfeld (DE); Edgar Dahl, Potsdam (DE); Andre Rosenthal, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Metagen Gesellschaft Fur Genomforschung MBH, Berlin (DE)

(21) Appl. No.: 10/131,487

(22) Filed: Apr. 25, 2002

Related U.S. Application Data
(62) Division of application No. 09/623,791, filed on Dec. 4, 2000.

(30) Foreign Application Priority Data
Mar. 10, 1998 (DE) 198 11 193.2

(51) Int. Cl. C12Q 1/68; G01N 33/574; C07H 21/04; C07K 14/47; C07K 16/30; C12P 21/02; C12N 5/06

(52) U.S. Cl. 435/6; 435/7.23; 435/69.1; 435/320.1; 435/325; 536/23.2; 530/350; 530/388.8

(57) ABSTRACT

Human nucleic acid sequences—mRNA, cDNA, genomic sequences—from tissue of prostate tumors, which code for gene products or parts thereof and their use, are described. In addition, the polypeptides that can be obtained by way of the sequences and their use are described.

The invention relates to human nucleic acid sequences—mRNA, cDNA, genomic sequences—from tissue of prostate tumors that code for gene products or parts thereof and their use.

The invention furthermore relates to polypeptides that can be obtained via the sequences and their use.

One very common type of cancer is prostate cancer, for control of which new therapies are necessary. Previously used therapies based on the blocking of hormone effects are very often ineffective after a few years since the tumor becomes independent of the hormone, i.e., it continues to grow and forms metastases without the action of hormones.

The cancer phenomenon often goes along with overexpression or underexpression of certain genes in degenerated cells, it still being unclear whether these altered expression rates are the cause or the result of the malignant transformation. Identification of these genes would be an important step for development of new therapies against cancer. Spontaneous formation of cancer is often preceded by a host of mutations. They can have the most varied effects on the expression pattern in the affected tissue, such as, e.g., underexpression or overexpression, but also expression of shortened genes. Several such changes due to these mutation cascades can ultimately lead to malignant degeneration. The complexity of these relationships makes an experimental approach very difficult.

A database that consists of so-called ESTs is used to look for tumor-related candidate genes, i.e., genes that can be regarded as the cause or the result of malignant degeneration of normal human tissue. ESTs (expressed sequence tags) are sequences of cDNAs, i.e., mRNAs transcribed in reverse, therefore molecules that reflect gene expression. The EST sequences are determined for normal and degenerated tissue. These databases are offered to some extent commercially by various companies. The ESTs of the LifeSeq database, which is used here, are generally between 150 and 350 nucleotides long. They represent a pattern that is unmistakable for a certain gene, although this gene is normally very much longer (>2000 nucleotides). By comparison of the expression patterns of normal and tumor tissue, ESTs can be identified that are important for tumor formation and proliferation (see FIG. 1). There is, however, the following problem: Since the EST sequences found can belong to different regions of an unknown gene due to different constructions of cDNA libraries, in this case a completely incorrect ratio of the occurrence of these ESTs in the respective tissue would arise. This would only be noticed when the complete gene is known and thus ESTs can be assigned to the same gene.

It has now been found that this error possibility can be reduced if all ESTs from the respective tissue type are assembled beforehand, before the expression patterns are compared to one another. Therefore, overlapping ESTs of the same gene were combined into longer sequences (see FIG. 1, FIG. 2a and FIG. 3). This lengthening and thus coverage of an essentially larger gene area in each of the respective bases are intended to largely avoid the above-described error. Since there were no existing software products for this purpose, programs for assembling genomic sections were employed which were used modified and to which our own programs were added. A flowchart of the assembly procedure is shown in FIG. 2b1-2b4.

Nucleic acid sequences Seq. ID No. 3, 4, 6, 7, 10, 12, 13, 15, 17-24, 26, 27, 29, 31-34, 36, 37, 39, 40, 44-53; 142, 144-164 which play a part as candidate genes in prostate cancer have now been found.

Nucleic acid sequences Seq. ID No. 3, 4, 6; 10, 12, 15, 17-24, 27, 29, 31-34, 36, 39, 44-53; 142, 144-164 are of special interest.

The invention thus relates to nucleic acid sequences that code a gene product or a part thereof, comprising

a) a nucleic acid sequence selected from the group of nucleic acid sequences Seq. ID No. 3, 4, 6; 10, 12, 15, 17-24, 27, 29, 31-34, 36, 39, 44-53; 142, 144-164.

b) an allelic variation of the nucleic acid sequences named under a) or

c) a nucleic acid sequence that is complementary to the nucleic acid sequences named under a) or b).

The invention furthermore relates to a nucleic acid sequence according to one of the sequences Seq. ID No. 3, 4, 6; 10, 12, 15, 17-24, 27, 29, 31-34, 36, 39, 44-53; 142, 144-164 or a complementary or allelic version thereof and the nucleic acid sequences thereof that have 90% to 95% homology to a human nucleic acid sequence.

The invention also relates to nucleic acid sequences Seq. ID No. 3-53; 142, 144-164, which are expressed elevated in the prostate tumor tissue.

The invention furthermore relates to nucleic acid sequences comprising part of the aforementioned nucleic acid sequences in such a sufficient amount that they hybridize with sequences Seq. ID No. 3, 4, 6; 10, 12, 15, 17-24, 27, 29, 31-34, 36, 39, 44-53; 142, 144-164.

The nucleic acid sequences according to the invention generally have a length of at least 50 to 2000 bp, preferably a length of at least 150 to 1700 bp.

With the partial sequences Seq. ID No. 3, 4, 6; 10, 12, 15, 17-24, 27, 29, 31-34, 36, 39, 44-53; 142, 144-164 according to the invention, expression cassettes can also be built using current process practice, whereby on the cassette at least one of the nucleic acid sequences according to the invention is combined with at least one control or regulator sequence known to one skilled in the art, such as, e.g., a suitable promoter. The sequences according to the invention can be inserted in a sense or antisense orientation.

A large number of expression cassettes or vectors and promoters, which can be used, are known in the literature.

Expression cassettes or vectors are defined as the following: 1. bacterial, such as, e.g., phagescript, pBs, ΦX174, pBluescript SK, pBs KS, pNH8a, pNH16a, pNH18a, pNH46a (Stratagene), pTrc99A, pKK223-3, pKK233-3, pDR540, pRIT5 (Pharmacia), 2. eukaryotic, such as, e.g., pWLneo, pSV2cat, pOG44, pXT1, pSG (Stratagene), pSVK3, pBPV, pMSG, pSVL (Pharmacia).

A control or regulatory sequence is defined as suitable promotors. Here two preferred vectors are the pKK232-8 and the PCM7 vector. In particular the following promoters are intended: lacI, lacZ, T3, T7, gpt, lambda P_{R}, trc, CMV, HSV thymidine-kinase, SV40, LTRs from retrovirus and mouse metallothionein-I.

The DNA sequences on the expression cassette can code a fusion protein, which comprises a known protein and a bioactive polypeptide fragment.

The expression cassettes are likewise the subject matter of this invention.

The nucleic acid fragments according to the invention can be used to produce full-length genes. The obtainable genes are likewise the subject matter of this invention.

The invention also relates to the use of the nucleic acid sequences according to the invention and the gene fragments obtainable from use.

The nucleic acid sequences according to the invention can be moved into host cells with suitable vectors in which as the heterologous part the genetic information which is expressed and which is contained on the nucleic acid fragments is located.

The host cells containing the nucleic acid fragments are likewise the subject matter of this invention.

Suitable host cells are, e.g., prokaryotic cell systems such as E. coli or eukaryotic cell systems such as animal or human cells or yeasts.

The nucleic acid sequences according to the invention can be used in the sense or antisense form.

Production of polypeptides or their fragments is done by cultivation of the host cells according to current cultivation methods and subsequent isolation and purification of the peptides or fragments, likewise using current methods. The invention furthermore relates to nucleic acid sequences, which code at least a partial sequence of a bioactive polypeptide.

Furthermore, this invention relates to polypeptide partial sequences, so-called ORF (open-reading-frame) peptides according to the sequence protocols Seq. ID No. 57-61, 64, 66-68, 70-71, 73-83, 85-87, 89-90, 92, 94, 95, 97, 101-141, 143 and 165-199.

The invention furthermore relates to polypeptide sequences that have at least 80% homology, especially 90% homology, to the polypeptide partial sequences according to the invention of Seq. ID No. 57-61, 64, 66-68, 70-71, 73-83, 85-87, 89-90, 92, 94, 95, 97, 101-141, 143 and 165-199.

The invention also relates to antibodies that are directed against a polypeptide or a fragment, which are coded by the nucleic acids of sequences Seq. ID No. 3, 4, 6; 10, 12, 15, 17-24, 27, 29, 31-34, 36, 39, 44-53; 142, 144-164 according to the invention.

Antibodies are defined especially as monoclonal antibodies.

The invention also relates to phage display proteins, which are directed against a polypeptide or a fragment, and which are coded by the nucleic acids of sequences Seq. ID No. 57-61, 64, 66-68, 70-71, 73-83, 85-87, 89-90, 92, 94, 95, 97, 101-141, 143 and 165-199 according to the invention.

The polypeptides of sequences Seq. ID No. 57-61, 64, 66-68, 70-71, 73-83, 85-87, 89-90, 92, 94, 95, 97, 101-141, 143 and 165-199 according to the invention can also be used as tools for finding active agents against prostate cancer; this is likewise the subject matter of this invention.

Likewise the subject matter of this invention is the use of nucleic acid sequences according to sequences Seq. ID No. 3, 4, 6; 10, 12, 15, 17-24, 27, 29, 31-34, 36, 39, 44-53; 142, 144-164 for expression of polypeptides, which can also be used as tools for finding active agents against prostate cancer.

The invention also relates to the use of the found polypeptide partial sequences Seq. ID No. 57-61, 64, 66-68, 70-71, 73-83, 85-87, 89-90, 92, 94, 95, 97, 101-141, 143 and 165-199 as pharmaceutical agents in gene therapy for treatment of prostate cancer, or to produce a pharmaceutical agent for treatment of prostate cancer.

The invention also relates to pharmaceutical agents that contain at least one polypeptide partial sequence of Seq. ID No. 57-61, 64, 66-68, 70-71, 73-83, 85-87, 89-90, 92, 94, 95, 97, 101-141, 143 and 165-199.

The nucleic acid sequences found according to the invention can also be genomic or mRNA sequences.

The invention also relates to genomic genes, their promoters, enhancers, silencers, exon structure, intron structure and their splice variants, obtainable from cDNAs of sequences Seq. ID No. 3, 4, 6; 10, 12, 15, 17-24, 27, 29, 31-34, 36, 39, 44-53; 142, 144-164 and their use together with suitable regulatory elements, such as suitable promoters and/or enhancers.

With the nucleic acids according to the invention (cDNA sequences), genomic BAC, PAC and Cosmid libraries are screened and specifically human clones are isolated via complementary base pairing (hybridization). The BAC, PAC and Cosmid clones isolated in this way are hybridized using fluorescence-in-situ hybridization on metaphase chromosomes and the corresponding chromosome sections on which the corresponding genomic genes lie are identified. BAC, PAC and Cosmid clones are sequenced in order to clarify the corresponding genomic genes in their complete structure (promoters, enhancers, silencers, exons and introns). BAC, PAC and Cosmid clones can be used as independent molecules for gene transfer (see FIG. 5).

The invention also relates to BAC, PAC and Cosmid clones containing functional genes and their chromosomal localization according to sequences Seq. ID No. 3, 4, 6; 10, 12, 15, 17-24, 27, 29, 31-34, 36, 39, 44-53; 142, 144-164 for use as vehicles for gene transfer.

Meanings of Technical Terms and Abbreviations

| | |
|---|---|
| Nucleic acids = | Nucleic acids in this invention are defined as: |
| | mRNA, partial cDNA, full-length cDNA and genomic |
| | genes (chromosomes). |
| ORF = | Open Reading Frame, a defined sequence of amino |
| | acids that can be derived from the cDNA sequence. |
| Contig = | A set of DNA sequences that can be combined as a |
| | result of very great similarities into one |
| | sequence (consensus). |
| Singleton = | A contig that contains only one sequence. |
| Module = | Domain of a protein with a defined sequence that |
| | represents one structural unit and that occurs in |
| | different proteins |
| N = | selectively the nucleotide A, T, G or C |
| X = | selectively one of the 20 naturally occurring |
| | amino acids |

Explanation of the Alignment Parameters

minimal initial match=minimal initial identity area

maximum pads per read=maximum number of insertions

maximum percent mismatch=maximum deviation in %

### EXPLANATION OF FIGURES

FIG. 1 shows a systematic gene search in the Incyte LifeSeq database.

FIG. 2a shows the principle of EST assembling

FIG. 2b1-2b4 shows the entire principle of EST assembling

FIG. 3 shows the in-silico subtraction of gene expression in different tissues

FIG. 4a shows the determination of tissue-specific expression via electronic Northern

FIG. 4b shows the electronic Northern

FIG. 5 shows the isolation of genomic BAC and PAC clones.

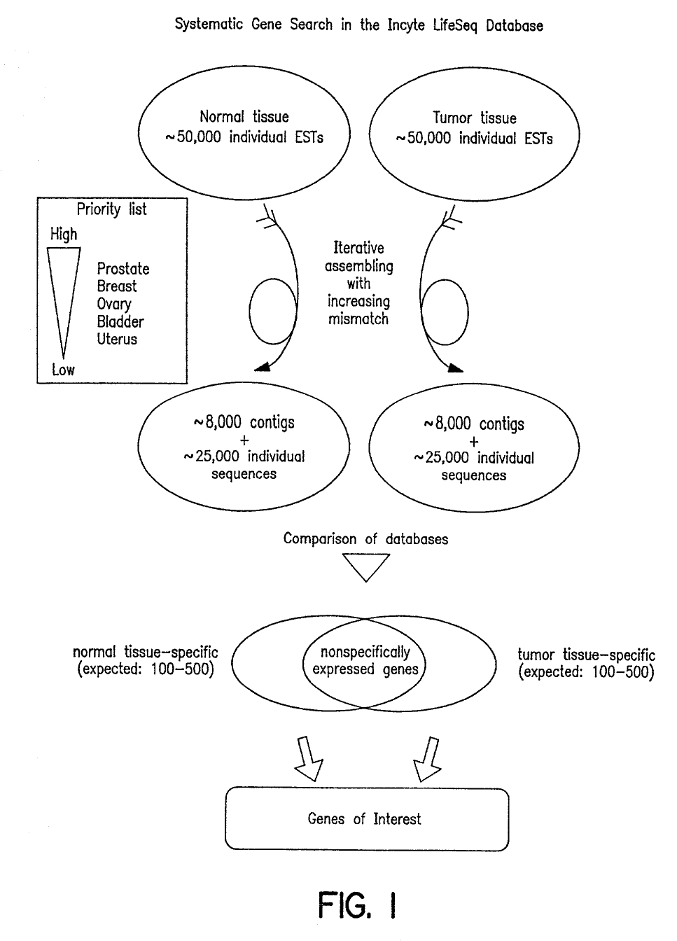

The following examples explain the production of the nucleic acid sequences according to the invention without limiting the invention to these examples and nucleic acid sequences.

### EXAMPLE 1

### Search for Tumor-Related Candidate Genes

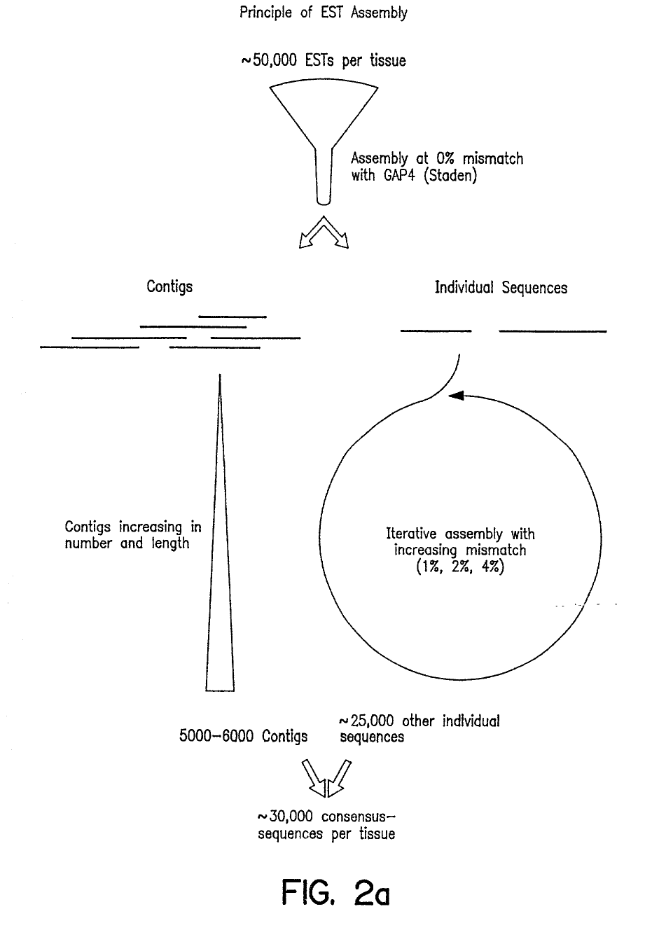

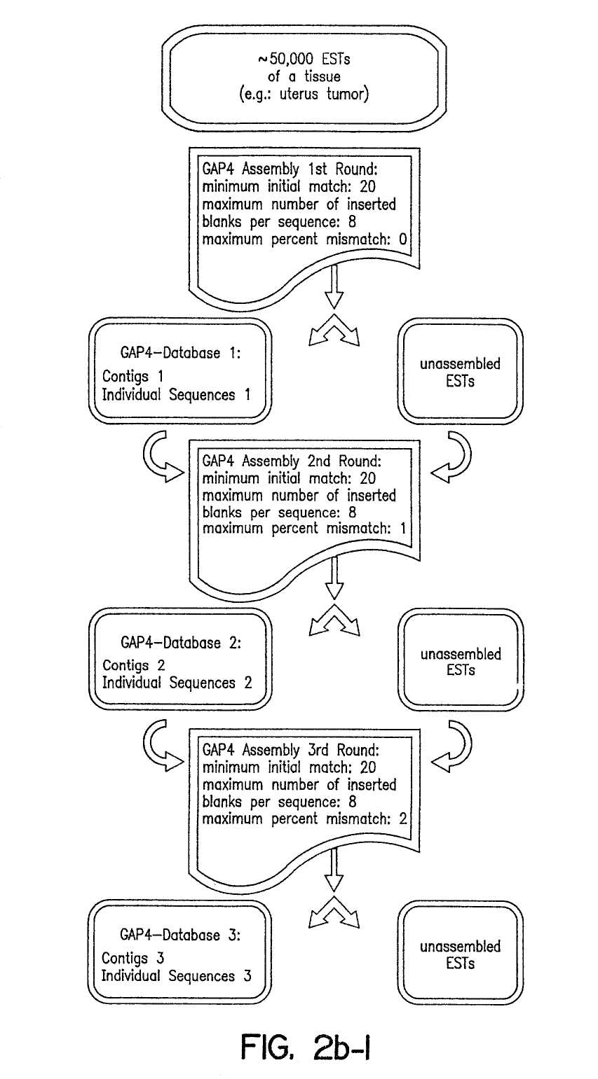

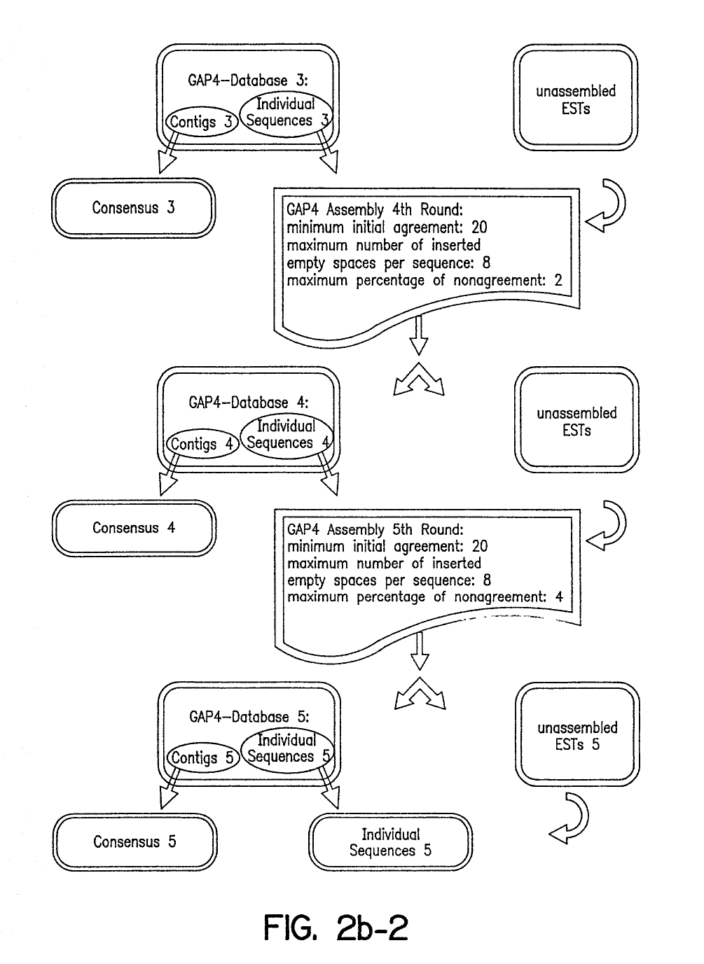

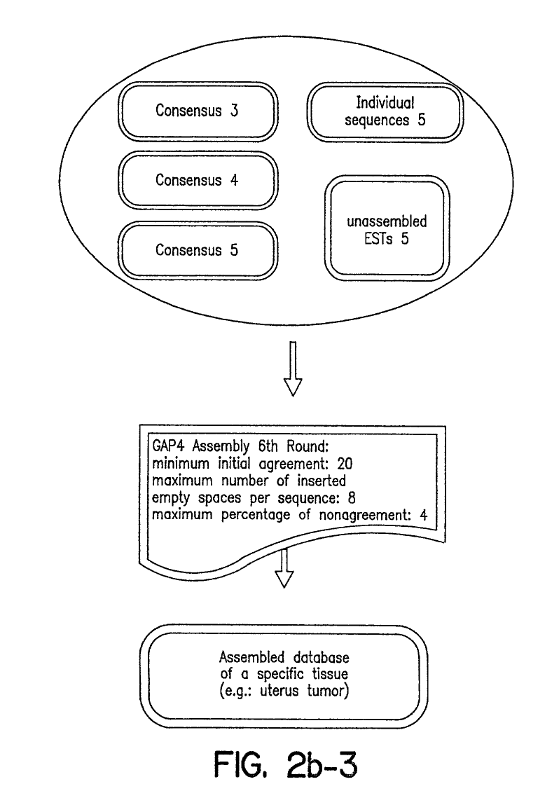

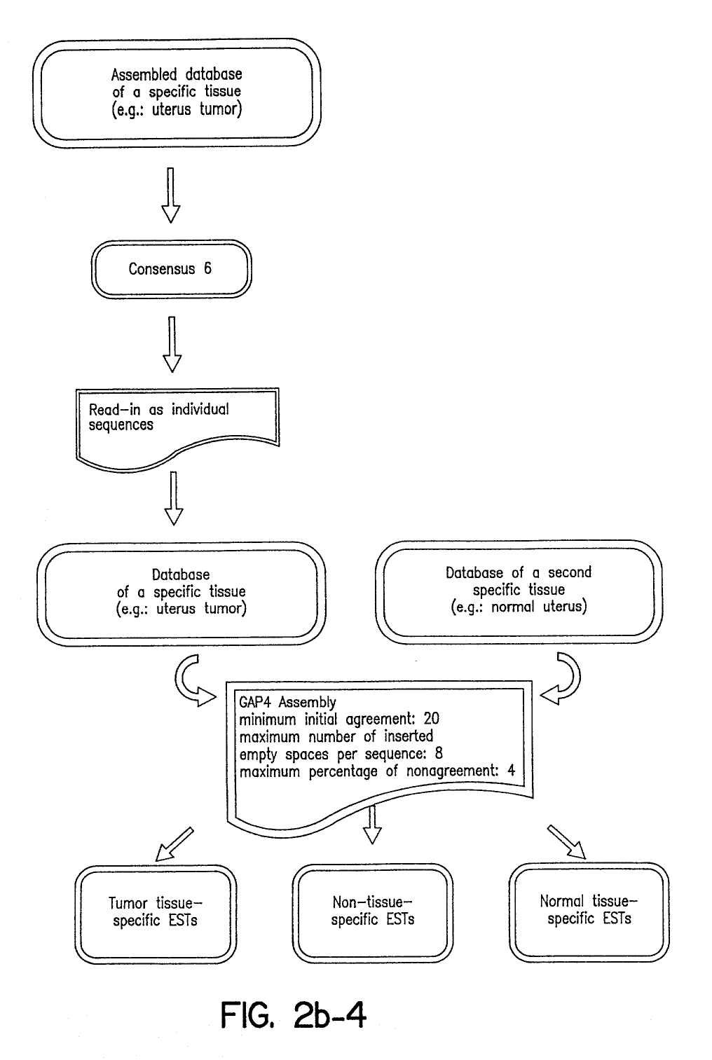

First, all ESTs of the corresponding tissue from the LifeSeq database (from October 1997) were extracted. They were then assembled by means of the GAP4 program of the Staden package with the parameters 0% mismatch, 8 pads per read and a minimal match of 20. The sequences (fails) not recorded in the GAP4 database were assembled first at 1% mismatch and then again at 2% mismatch with the database. Consensus sequences were computed from the contigs of the database, which consisted of more than one sequence (see FIG. 2a and 2b1-2b4).

The singletons of the database, which consisted of only one sequence, were re-assembled at 2% mismatch with the sequences not recorded in the GAP4 database. In turn, for the contigs the consensus sequences were determined. All other ESTs were re-assembled at 4% mismatch. The consensus sequences were extracted once again and finally assembled with the previous consensus sequences and the singletons and the sequences not recorded in the database at 4% mismatch. The consensus sequences were formed and used with the singletons and fails as the initial basis for tissue comparisons. This procedure ensured that among the parameters used all sequences represented gene regions independent of one another.

FIG. 2b1-2b4 illustrates the lengthening of the prostate tumor ESTs.

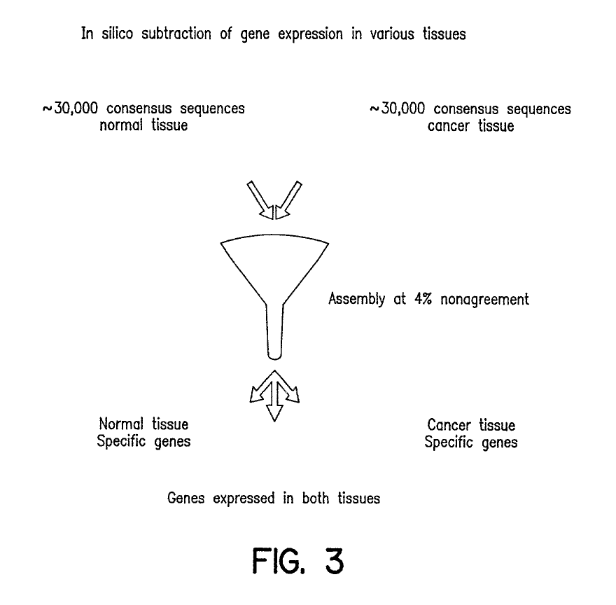

The sequences of the respective tissue assembled in this way were then compared to one another by means of the same program (see FIG. 3). To do this, first, all sequences of the first tissue were input into the database. (Therefore it was important that they were independent of one another).

Then all sequences of the second tissue were compared to all those of the first. The result was sequences that were specific to the first or the second tissue as well as those which occurred in both. In the latter, the ratio of the frequency of occurrence in the respective tissue was evaluated.

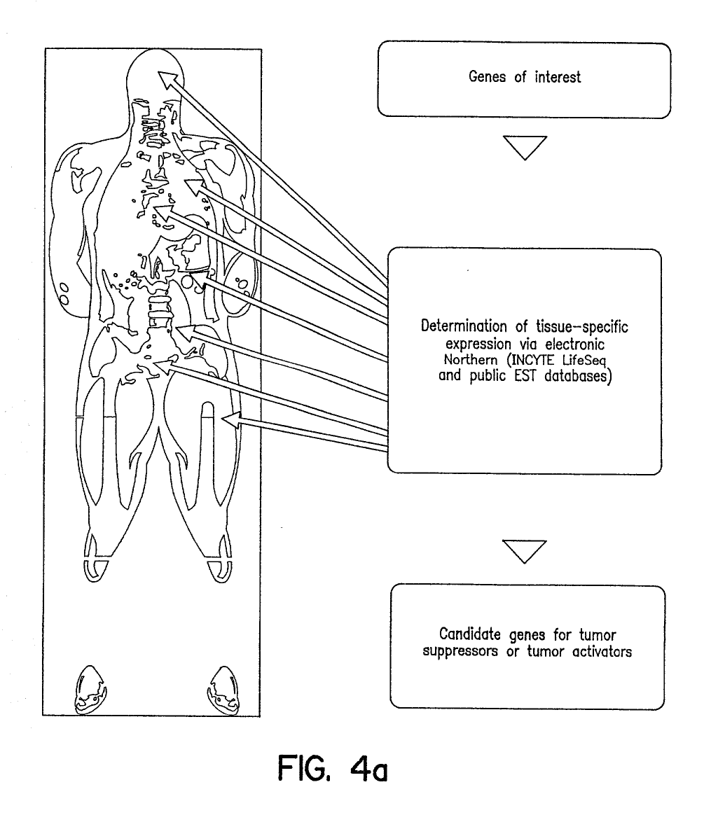

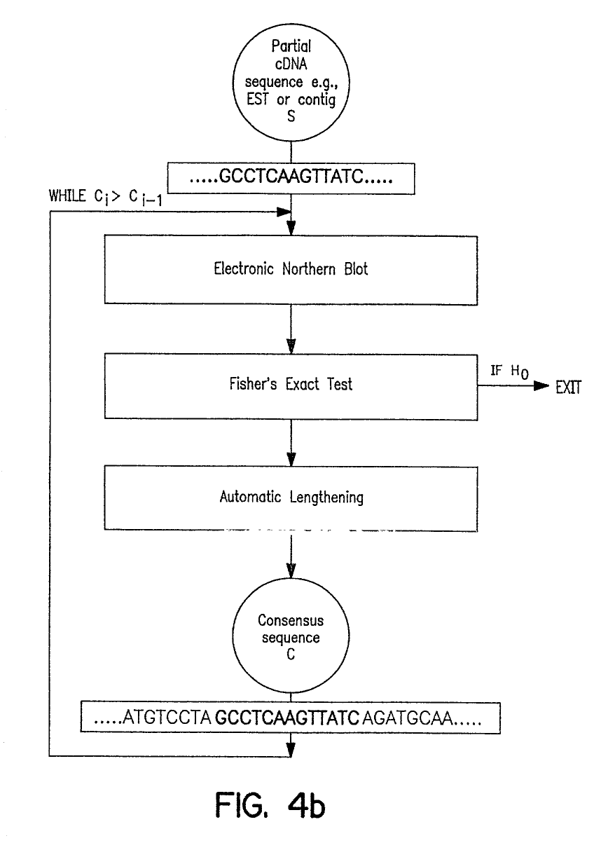

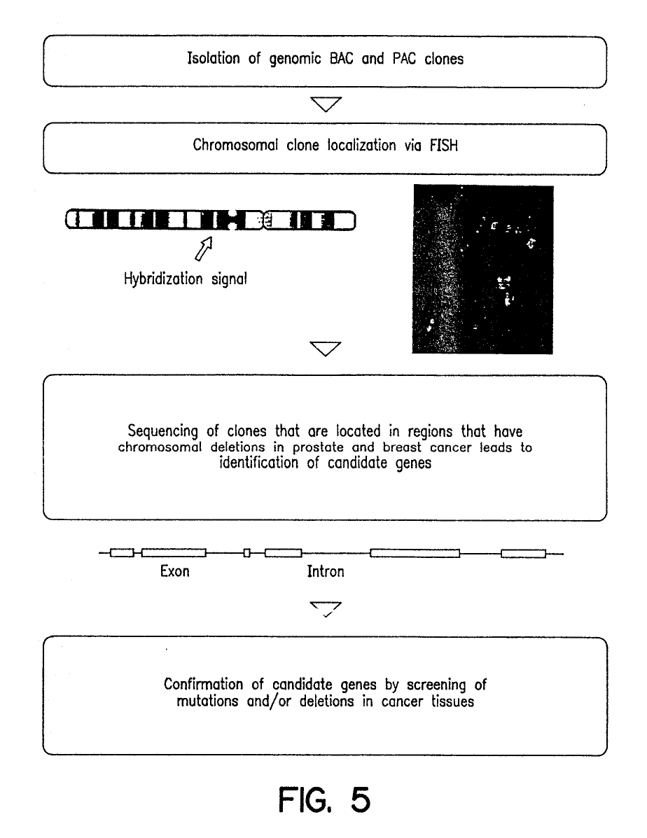

All sequences that occurred more than four times in one of the compared tissues in each case and all that occurred at least five times as often in one of the two tissues were further studied. These sequences were subjected to an electronic Northern (see Example 2.1), by which the distribution in all tumor and normal tissues was studied (see FIG. 4a and FIG. 4b). The relevant candidates were then lengthened using all Incyte ESTs and all ESTs of public databases (see example 3). Then the sequences and their translation into possible proteins were compared to all nucleotide and protein databases, and studied for possible regions that code for proteins.

### EXAMPLE 2

### Algorithm for Identification and Lengthening of Partial cDNA Sequences with Altered Expression Pattern

An algorithm for finding overexpressed or underexpressed genes will be explained below. The individual steps are also summarized in a flowchart for the sake of clarity (see FIG. 4b).

2.1. Electronic Northern Blot

By means of a standard program for homology search, e.g., BLAST (Altschul, S. F., Gish W., Miller, W., Myers, E. W. and Lipman, D. J. (1990) J. Mol. Biol. 215, 403-410), BLAST2 (Altschul S. F., Madden, T. L., Schaeffer, A. A., Zhang, J., Zhang, Z., Miller, W., and Lipman, D. J. (1997) Nucleic Acids Research 25 3389-3402) or FASTA (Pearson, W. R. and Lipman, D. J. (1988) Proc. Natl. Acad. Sci. USA 85 2444-2448) the homologous sequences in different EST libraries (private or public) arranged by tissues were determined for a partial DNA sequence S, e.g., an individual EST or a contig of ESTs. The (relative or absolute) tissue-specific occurrence frequencies of this partial sequence S which were determined in this way are called electronic Northern Blot.

2.1.1

Analogously to the procedure described under 2.1, the EST of the sequence Seq. ID No. 36 was found which occurs 5× more heavily in prostate tumor tissue than in the corresponding normal tissue.

The possible function of this gene region relates to the glutamate-binding subunit of the NMDA receptor.

The result is as follows:

| | NORMAL | TUMOR | RATIOS | |
|---|---|---|---|---|
| | % freq. | % freq. | N/T | T/N |
| Bladder | 0.0465 | 0.0204 | 2.2731 | 0.4399 |
| Breast | 0.0373 | 0.0283 | 1.3172 | 0.7592 |
| Ovary | 0.0091 | 0.0078 | 1.1686 | 0.8557 |
| Endocrine | 0.0292 | 0.0163 | 1.7861 | 0.5599 |
| tissue | | | | |
| Gastrointestinal | 0.0039 | 0.0286 | 0.1357 | 7.3686 |
| Brain | 0.0195 | 0.0350 | 0.5564 | 1.7973 |
| Hematopoetic | 0.0112 | 0.0000 | undef | 0.0000 |
| Skin | 0.0249 | 0.0000 | undef | 0.0000 |
| Hepatic | 0.0000 | 0.0000 | undef | undef |
| Heart | 0.0064 | 0.0137 | 0.4624 | 2.1624 |
| Testicles | 0.0061 | 0.0000 | undef | 0.0000 |
| Lungs | 0.0324 | 0.0142 | 2.2824 | 0.4381 |
| Stomach- | 0.0097 | 0.0307 | 0.3150 | 3.1748 |
| esophagus | | | | |
| Muscle- | 0.0069 | 0.0000 | undef | 0.0000 |
| skeleton | | | | |
| Kidneys | 0.0059 | 0.0274 | 0.2171 | 4.6066 |
| Pancreas | 0.0076 | 0.0166 | 0.4571 | 2.1876 |
| Penis | 0.0150 | 0.0800 | 0.1872 | 5.3424 |
| Prostate | 0.0071 | 0.0341 | 0.2097 | 4.7677 |
| Uterus | 0.0066 | 0.0000 | undef | 0.0000 |
| Breast- | 0.0036 | | | |
| hyperplasia | | | | |
| Small intestine | 0.0062 | | | |
| Prostatic | 0.0208 | | | |
| hyperplasia | | | | |
| Seminal vesicles | 0.0089 | | | |
| Sensory organs | 0.0000 | | | |
| White blood cells | 0.0131 | | | |

| | FETUS |
|---|---|
| | % freq. |
| Development | 0.0000 |
| Gastrointenstinal | 0.0092 |
| Brain | 0.0313 |
| Hematopoetic | 0.0000 |
| Heart blood vessels | 0.0000 |
| Lungs | 0.0111 |
| Kidneys | 0.0124 |
| Prostate | 0.0249 |
| Sensory organs | 0.0000 |
| | STANDARDIZED/ |
| | SUBTRACTED LIBRARIES |
| | % freq. |
| Breast | 0.0748 |
| Ovary uterus | 0.0388 |
| Endocrine tissue | 0.0000 |
| Fetal | 0.0012 |
| Gastrointestinal | 0.0244 |
| Hematopoetic | 0.0057 |
| Skin muscle | 0.0032 |
| Testicles | 0.0156 |
| Lungs | 0.0000 |
| Nerves | 0.0131 |
| Prostate | 0.0256 |
| Sensory organs | 0.0077 |

2.1.3

Analogously to the procedure described under 2.1, the EST of the sequence Seq. ID No. 39 was found which occurs 4.5× more heavily in prostate tumor tissue than in the corresponding normal tissue.

The possible function of this gene region relates to protein control (protein trafficking) in the endoplasmatic reticulum (ER)

The result is as follows:

| | NORMAL | TUMOR | RATIOS | |
|---|---|---|---|---|
| | % freq. | % freq. | N/T | T/N |
| Bladder | 0.0046 | 0.0051 | 0.9092 | 1.0998 |
| Breast | 0.0067 | 0.0022 | 3.0579 | 0.3270 |
| Ovary | 0.0091 | 0.0026 | 3.5059 | 0.2852 |
| Endocrine | 0.0036 | 0.0054 | 0.6698 | 1.4930 |
| tissue | | | | |
| Gastrointestinal | 0.0058 | 0.0095 | 0.6107 | 1.6375 |
| Brain | 0.0034 | 0.0033 | 1.0321 | 0.9689 |
| Hematopoetic | 0.0056 | 0.0000 | undef | 0.0000 |
| Skin | 0.0050 | 0.0000 | undef | 0.0000 |
| Hepatic | 0.0099 | 0.0000 | undef | 0.0000 |
| Heart | 0.0021 | 0.0000 | undef | 0.0000 |
| Testicles | 0.0061 | 0.0000 | undef | 0.0000 |
| Lungs | 0.0062 | 0.0047 | 1.3168 | 0.7594 |
| Stomach | 0.0193 | 0.0000 | undef | 0.0000 |
| esophagus | | | | |
| Muscle- | 0.0051 | 0.0060 | 0.8565 | 1.1675 |
| skeleton | | | | |
| Kidneys | 0.0000 | 0.0000 | undef | undef |
| Pancreas | 0.0038 | 0.0055 | 0.6857 | 1.4584 |
| Penis | 0.0000 | 0.0000 | undef | undef |
| Prostate | 0.0024 | 0.0106 | 0.2237 | 4.4697 |
| Uterus | 0.0017 | 0.0000 | undef | 0.0000 |
| Breast- | 0.0000 | | | |
| hyperplasia | | | | |
| Small | 0.0093 | | | |
| intestine | | | | |
| Prostatic | 0.0089 | | | |
| hyperplasia | | | | |
| Seminal- | 0.0000 | | | |
| vesicles | | | | |
| Sensory organs | 0.0000 | | | |
| White blood | 0.0026 | | | |
| cells | | | | |

| | FETUS |
|---|---|
| | % freq. |
| Development | 0.0000 |
| Gastrointenstinal | 0.0062 |
| Brain | 0.0000 |
| Hematopoetic | 0.0079 |
| Heart blodd vessels | 0.0000 |
| Lungs | 0.0074 |
| Kidneys | 0.0247 |
| Prostate | 0.0000 |
| Sensory organs | 0.0000 |
| | STANDARDIZED/ |
| | SUBTRACTED LIBRARIES |
| | % freq. |
| Breast | 0.0000 |
| Ovary uterus | 0.0091 |
| Endocrine tissue | 0.0000 |
| Fetal | 0.0017 |
| Gastrointestinal | 0.0122 |
| Hematopoetic | 0.0000 |
| Skin muscle | 0.0162 |
| Testicles | 0.0078 |
| Lungs | 0.0082 |
| Nerves | 0.0020 |
| Prostate | 0.0128 |
| Sensory organs | 0.0000 |

2.1.4

Analogously to the procedure described under 2.1, the EST of the sequence Seq. ID No. 40 was found which occurs 8× more heavily in prostate tumor tissue than in the corresponding normal tissue.

The possible function of this gene region relates to the regulation of protein kinase.

The result is as follows:

| | NORMAL | TUMOR | RATIOS | |
|---|---|---|---|---|
| | % freq. | % freq. | N/T | T/N |
| Bladder | 0.0000 | 0.0000 | undef | undef |
| Breast | 0.0120 | 0.0763 | 0.1573 | 6.3588 |
| Ovary | 0.0608 | 0.0234 | 2.5969 | 0.3851 |
| Endocrine | 0.0073 | 0.0245 | 0.2977 | 3.3593 |
| tissue | | | | |
| Gastrointestinal | 0.0872 | 0.1000 | 0.8724 | 1.1462 |
| Brain | 0.0017 | 0.0000 | undef | 0.0000 |
| Hematopoetic | 0.0014 | 0.0000 | undef | 0.0000 |
| Skin | 0.0000 | 0.0000 | undef | undef |
| Hepatic | 0.0149 | 0.0388 | 0.3826 | 2.6139 |
| Heart | 0.0011 | 0.0000 | undef | 0.0000 |
| Testicles | 0.0000 | 0.0000 | undef | undef |
| Lungs | 0.0050 | 0.0355 | 0.1405 | 7.1196 |
| Stomach | 0.0000 | 0.0230 | 0.0000 | undef |
| esophagus | | | | |
| Muscle- | 0.0000 | 0.0000 | undef | undef |
| skeleton | | | | |
| Kidneys | 0.0000 | 0.0000 | undef | undef |
| Pancreas | 0.0000 | 0.0166 | 0.0000 | undef |
| Penis | 0.0000 | 0.0000 | undef | undef |
| Prostate | 0.0119 | 0.0958 | 0.1243 | 8.0455 |
| Uterus | 0.0017 | 0.0214 | 0.0774 | 12.9263 |
| Breast- | 0.0073 | | | |
| hyperplasia | | | | |
| Small- | 0.0436 | | | |
| intestine | | | | |
| Prostatic | 0.0119 | | | |
| hyperplasia | | | | |
| Seminal- | 0.0089 | | | |
| vesicles | | | | |
| Sensory organs | 0.0470 | | | |
| White blood | 0.0009 | | | |
| cells | | | | |

| | FETUS |
|---|---|
| | % freq. |
| Development | 0.0000 |
| Gastrointenstinal | 0.0247 |
| Brain | 0.0000 |
| Hematopoetic | 0.0000 |
| Heart blood vessels | 0.0000 |
| Lungs | 0.0000 |
| Kidneys | 0.0000 |
| Prostate | 0.0000 |
| Sensory organs | 0.0000 |
| | STANDARDIZED/ |
| | SUBTRACTED LIBRARIES |
| | % freq. |
| Breast | 0.0000 |
| Ovary uterus | 0.0205 |
| Endocrine tissue | 0.0000 |
| Fetal | 0.0052 |
| Gastrointestinal | 0.0366 |
| Hematopoetic | 0.0000 |
| Skin muscle | 0.0000 |
| Testicles | 0.0000 |
| Lungs | 0.0000 |
| Nerves | 0.0000 |
| Prostate | 0.0321 |
| Sensory organs | 0.0000 |

The following northerns were also found in a similar procedure:

Electronic Northern Blot for SEQ. ID. NO: 3

| | NORMAL | TUMOR | RATIOS | |
|---|---|---|---|---|
| | % freq. | % freq. | N/T | T/N |
| Bladder | 0.0046 | 0.0128 | 0.3637 | 2.7495 |
| Breast | 0.0293 | 0.0087 | 3.3637 | 0.2973 |
| Ovary | 0.0122 | 0.0416 | 0.2922 | 3.4228 |
| Endocrine | 0.0201 | 0.0245 | 0.8186 | 1.2216 |
| tissue | | | | |
| Gastrointestinal | 0.0252 | 0.0571 | 0.4411 | 2.2673 |
| Brain | 0.0161 | 0.0131 | 1.2257 | 0.8159 |
| Hematopoetic | 0.0084 | 0.1135 | 0.0739 | 13.5274 |
| Skin | 0.0099 | 0.0000 | undef | 0.0000 |
| Hepatic | 0.0000 | 0.0388 | 0.0000 | undef |
| Heart | 0.0180 | 0.0000 | undef | 0.0000 |
| Testicles | 0.0122 | 0.0000 | undef | 0.0000 |
| Lungs | 0.0162 | 0.0189 | 0.8559 | 1.1683 |
| Stomach- | 0.0000 | 0.0153 | 0.0000 | undef |
| esophogas | | | | |
| Muscle- | 0.0069 | 0.0000 | undef | 0.0000 |
| skeleton | | | | |
| Kidneys | 0.0149 | 0.0342 | 0.4342 | 2.3033 |
| Pancreas | 0.0265 | 0.0276 | 0.9599 | 1.0417 |
| Penis | 0.0180 | 0.0267 | 0.6739 | 1.4840 |
| Prostate | 0.0048 | 0.0149 | 0.3196 | 3.1288 |
| Uterus | 0.0066 | 0.0142 | 0.4642 | 2.1544 |
| Breast- | 0.0291 | | | |
| hyperplasia | | | | |
| Small- | 0.0093 | | | |
| intestine | | | | |
| Prostate- | 0.0089 | | | |
| hyperplasia | | | | |
| Seminal- | 0.0178 | | | |
| vesicles | | | | |
| Sensory organs | 0.0000 | | | |
| White blood | 0.0148 | | | |
| cells | | | | |

| | FETUS |
|---|---|
| | % freq. |
| Development | 0.0307 |
| Gastrointestinal | 0.0062 |
| Brain | 0.0375 |
| Hematopoetic | 0.0079 |
| Heart blood vessels | 0.0164 |
| Lungs | 0.0037 |
| Kidneys | 0.0062 |
| Prostate | 0.0499 |
| Sensory organs | 0.0000 |
| | STANDARDIZED/ |
| | SUBTRACTED LIBRARIES |
| | % freq. |
| Breast | 0.0136 |
| Ovary uterus | 0.0000 |
| Endocrine tissue | 0.0000 |
| Fetal | 0.0064 |
| Gastrointestinal | 0.0244 |
| Hematopoetic | 0.0114 |
| Skin muscle | 0.0000 |
| Testicles | 0.0000 |
| Lungs | 0.0328 |
| Nerves | 0.0120 |
| Prostate | 0.0064 |
| Sensory organs | 0.0000 |

Electronic Northern Blot for SEQ. ID. NO: 4

| | NORMAL | TUMOR | RATIOS | |
|---|---|---|---|---|
| | % freq. | % freq. | N/T | T/N |
| Bladder | 0.0046 | 0.0153 | 0.3031 | 3.2995 |
| Breast | 0.0027 | 0.0044 | 0.6116 | 1.6351 |
| Ovary | 0.0030 | 0.0026 | 1.1686 | 0.8557 |
| Endocrine- | 0.0018 | 0.0082 | 0.2233 | 4.4791 |
| tissue | | | | |
| Gastrointestinal | 0.0000 | 0.0048 | 0.0000 | undef |
| Brain | 0.0000 | 0.0099 | 0.0000 | undef |
| Hematopoetic | 0.0070 | 0.0000 | undef | 0.0000 |
| Skin | 0.0050 | 0.0000 | undef | 0.0000 |
| Hepatic | 0.0000 | 0.0000 | undef | undef |
| Heart | 0.0042 | 0.0000 | undef | 0.0000 |
| Testicles | 0.0061 | 0.0000 | undef | 0.0000 |
| Lungs | 0.0037 | 0.0024 | 1.5801 | 0.6329 |
| Stomach- | 0.0097 | 0.0000 | undef | 0.0000 |
| esophagus | | | | |
| Muscle- | 0.0034 | 0.0120 | 0.2855 | 3.5025 |
| skeleton | | | | |
| Kidneys | 0.0059 | 0.0068 | 0.8683 | 1.1517 |
| Pancreas | 0.0019 | 0.0055 | 0.3428 | 2.9168 |
| Penis | 0.0000 | 0.0267 | 0.0000 | undef |
| Prostate | 0.0048 | 0.0192 | 0.2486 | 4.0228 |
| Uterus | 0.0083 | 0.0000 | undef | 0.0000 |
| Breast- | 0.0000 | | | |
| hyperplasia | | | | |
| Small- | 0.0000 | | | |
| intestine | | | | |
| Prostate- | 0.0030 | | | |
| hyperplasia | | | | |
| Seminal- | 0.0267 | | | |
| vesicles | | | | |
| Sensory-organs | 0.0000 | | | |
| White blood | 0.0000 | | | |
| cells | | | | |

| | FETUS |
|---|---|
| | % freq. |
| Development | 0.0000 |
| Gastrointestinal | 0.0000 |
| Brain | 0.0000 |
| Hematopoetic | 0.0000 |
| Heart blood | 0.0000 |
| vessels | |
| Lungs | 0.0000 |
| Kidneys | 0.0000 |
| Prostate | 0.0000 |
| Sensory organs | 0.0000 |
| | STANDARDIZED/ |
| | SUBTRACTED LIBRARIES |
| | % freq. |
| Breast | 0.0000 |
| Ovary uterus | 0.0068 |
| Endocrine tissue | 0.0000 |
| Fetal | 0.0006 |
| Gastrointestinal | 0.0000 |
| Hematopoetic | 0.0000 |
| Skin muscle | 0.0000 |
| Testicles | 0.0000 |
| Lungs | 0.0082 |
| Nerves | 0.0040 |
| Prostate | 0.0256 |
| Sensory organs | 0.0000 |

Electronic Northern Blot for SEQ. ID. NO: 6

| | NORMAL | TUMOR | RATIOS | |
|---|---|---|---|---|
| | % freq. | % freq. | N/T | T/N |
| Bladder | 0.0046 | 0.0026 | 1.8185 | 0.5499 |
| Breast | 0.0027 | 0.0000 | undef | 0.0000 |
| Ovary | 0.0000 | 0.0078 | 0.0000 | undef |
| Endocrine | 0.0000 | 0.0027 | 0.0000 | undef |
| tissue | | | | |
| Gastrointestinal | 0.0039 | 0.0000 | undef | 0.0000 |
| Brain | 0.0025 | 0.0000 | undef | 0.0000 |
| Hematopoetic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lungs | 0.0012 | 0.0000 | undef | 0.0000 |
| Stomach- | 0.0193 | 0.0077 | 2.5198 | 0.3968 |
| esophagus | | | | |
| Muscle- | 0.0000 | 0.0000 | undef | undef |
| skeleton | | | | |
| Kidneys | 0.0059 | 0.0000 | undef | 0.0000 |
| Pancreas | 0.0000 | 0.0000 | undef | undef |
| Penis | 0.0030 | 0.0000 | undef | 0.0000 |
| Prostate | 0.0024 | 0.0170 | 0.1398 | 7.1516 |
| Uterus | 0.0066 | 0.0000 | undef | 0.0000 |
| Breast- | 0.0036 | | | |
| hyperplasia | | | | |
| Small- | 0.0000 | | | |
| Intestine | | | | |
| Prostate- | 0.0030 | | | |
| hyperplasia | | | | |
| Seminal- | 0.0000 | | | |
| vesicles | | | | |
| Sensory organs | 0.0000 | | | |
| White blood | 0.0000 | | | |
| cells | | | | |

| | FETUS |
|---|---|
| | % freq. |
| Development | 0.0000 |
| Gastrointestinal | 0.0000 |
| Brain | 0.0000 |
| Hematopoetic | 0.0039 |
| Heart blood vessels | 0.0000 |
| Lungs | 0.0000 |
| Kidneys | 0.0124 |
| Prostate | 0.0000 |
| Sensory organs | 0.0000 |
| | STANDARDIZED/ |
| | SUBTRACTED LIBRARIES |
| | % freq. |
| Breast | 0.0000 |
| Ovary uterus | 0.0023 |
| Endocrine tissue | 0.0000 |
| Fetal | 0.0000 |
| Gastrointestinal | 0.0000 |
| Hematopoetic | 0.0000 |
| Skin muscle | 0.0000 |
| Testicles | 0.0000 |
| Lungs | 0.0000 |
| Nerves | 0.0000 |
| Prostate | 0.0064 |
| Sensory organs | 0.0000 |

Electronic Northern Blot for SEQ. ID. NO: 7

| | NORMAL | TUMOR | RATIOS | |
|---|---|---|---|---|
| | % freq. | % freq. | N/T | T/N |
| Bladder | 0.0000 | 0.0153 | 0.0000 | undef |
| Breast | 0.0027 | 0.0109 | 0.2446 | 4.0878 |
| Ovary | 0.0000 | 0.0000 | undef | undef |
| Endocrine- | 0.0036 | 0.0027 | 1.3396 | 0.7465 |
| tissue | | | | |
| Gastrointestinal | 0.0097 | 0.0000 | undef | 0.0000 |
| Brain | 0.0008 | 0.0011 | 0.7741 | 1.2918 |
| Hematopoetic | 0.0028 | 0.0000 | undef | 0.0000 |
| Skin | 0.0000 | 0.0000 | undef | undef |
| Hepatic | 0.0099 | 0.0129 | 0.7651 | 1.3069 |
| Heart | 0.0011 | 0.0000 | undef | 0.0000 |
| Testicles | 0.0000 | 0.0000 | undef | undef |
| Lungs | 0.0012 | 0.0000 | undef | 0.0000 |
| Stomach- | 0.0000 | 0.0000 | undef | undef |
| esophagus | | | | |
| Muscle- | 0.0000 | 0.0000 | undef | undef |
| skeleton | | | | |
| Kidneys | 0.0000 | 0.0000 | undef | undef |
| Pancreas | 0.0019 | 0.0055 | 0.3428 | 2.9168 |
| Penis | 0.0000 | 0.0000 | undef | undef |
| Prostate | 0.0000 | 0.0064 | 0.0000 | undef |
| Uterus | 0.0017 | 0.0000 | undef | 0.0000 |
| Breast- | 0.0036 | | | |
| hyperplasia | | | | |
| Small- | 0.0031 | | | |
| intestine | | | | |
| Prostate- | 0.0030 | | | |
| hyperplasia | | | | |
| Seminal- | 0.0000 | | | |
| vesicles | | | | |
| Sensory organs | 0.0000 | | | |
| White blood | 0.0009 | | | |
| cells | | | | |

| | FETUS |
|---|---|
| | % freq. |
| Development | 0.0000 |
| Gastrointestinal | 0.0062 |
| Brain | 0.0063 |
| Hematopoetic | 0.0000 |
| Heart blood vessels | 0.0041 |
| Lungs | 0.0037 |
| Kidneys | 0.0124 |
| Prostate | 0.0000 |
| Sensory organs | 0.0000 |
| | STANDARDIZED/ |
| | SUBTRACTED LIBRARIES |
| | % freq. |
| Breast | 0.0000 |
| Ovary uterus | 0.0023 |
| Endocrine tissue | 0.0000 |
| Fetal | 0.0029 |
| Gastrointestinal | 0.0000 |
| Hematopoetic | 0.0228 |
| Skin muscle | 0.0000 |
| Testicles | 0.0000 |
| Lungs | 0.0000 |
| Nerves | 0.0010 |
| Prostate | 0.0000 |
| Sensory organs | 0.0000 |

Electronic Northern Blot for SEQ. ID. NO: 10

| | NORMAL | TUMOR | RATIOS | |
|---|---|---|---|---|
| | % freq. | % freq. | N/T | T/N |
| Bladder | 0.0093 | 0.0051 | 1.8185 | 0.5499 |
| Breast | 0.0027 | 0.0000 | undef | 0.0000 |
| Ovary | 0.0000 | 0.0026 | 0.0000 | undef |
| Endocrine- | 0.0000 | 0.0000 | undef | undef |
| tissue | | | | |
| Gastrointestinal | 0.0039 | 0.0000 | undef | 0.0000 |
| Brain | 0.0076 | 0.0066 | 1.1612 | 0.8612 |
| Hematopoetic | 0.0042 | 0.0000 | undef | 0.0000 |
| Skin | 0.0050 | 0.0000 | undef | 0.0000 |
| Hepatic | 0.0000 | 0.0000 | undef | undef |
| Heart | 0.0011 | 0.0275 | 0.0385 | 25.9489 |
| Testicles | 0.0000 | 0.0000 | undef | undef |
| Lungs | 0.0000 | 0.0047 | 0.0000 | undef |
| Stomach- | 0.0000 | 0.0000 | undef | undef |
| esophagus | | | | |
| Muscle- | 0.0017 | 0.0000 | undef | 0.0000 |
| skeleton | | | | |
| Kidneys | 0.0030 | 0.0000 | undef | 0.0000 |
| Pancreas | 0.0038 | 0.0055 | 0.6857 | 1.4584 |
| Penis | 0.0000 | 0.0000 | undef | undef |
| Prostate | 0.0048 | 0.0106 | 0.4475 | 2.2349 |
| Uterus | 0.0017 | 0.0000 | undef | 0.0000 |
| Breast- | 0.0000 | | | |
| hyperplasia | | | | |
| Small- | 0.0156 | | | |
| intestine | | | | |
| Prostate- | 0.0000 | | | |
| hyperplasia | | | | |
| Seminal- | 0.0089 | | | |
| vesicles | | | | |
| Sensory organs | 0.0118 | | | |
| White blood | 0.0000 | | | |
| cells | | | | |

| | FETUS |
|---|---|
| | % freq. |
| Development | 0.0000 |
| Gastrointestinal | 0.0031 |
| Brain | 0.0000 |
| Hematopoetic | 0.0000 |
| Heart blood vessels | 0.0000 |
| Lungs | 0.0000 |
| Kidneys | 0.0000 |
| Prostate | 0.0000 |
| Sensory organs | 0.0000 |
| | STANDARDIZED/ |
| | SUBTRACTED LIBRARIES |
| | % freq. |
| Breast | 0.0000 |
| Ovary uterus | 0.0160 |
| Endocrine tissue | 0.0000 |
| Fetal | 0.0058 |
| Gastrointestinal | 0.0000 |
| Hematopoetic | 0.0114 |
| Skin muscle | 0.0000 |
| Testicles | 0.0000 |
| Lungs | 0.0000 |
| Nerves | 0.0060 |
| Prostate | 0.0000 |
| Sensory organs | 0.0155 |

Electronic Northern Blot for SEQ. ID. NO: 12

| | NORMAL | TUMOR | RATIOS | |
|---|---|---|---|---|
| | % freq. | % freq. | N/T | T/N |
| Bladder | 0.0046 | 0.0128 | 0.3637 | 2.7495 |
| Breast | 0.0080 | 0.0022 | 3.6695 | 0.2725 |
| Ovary | 0.0061 | 0.0026 | 2.3372 | 0.4279 |
| Endocrine- | 0.0036 | 0.0082 | 0.4465 | 2.2395 |
| tissue | | | | |
| Gastrointestinal | 0.0058 | 0.0048 | 1.2214 | 0.8187 |
| Brain | 0.0025 | 0.0077 | 0.3318 | 3.0142 |
| Hematopoetic | 0.0084 | 0.0000 | undef | 0.0000 |
| Skin | 0.0000 | 0.0000 | undef | undef |
| Hepatic | 0.0000 | 0.0000 | undef | undef |
| Heart | 0.0042 | 0.0137 | 0.3083 | 3.2436 |
| Testicles | 0.0000 | 0.0000 | undef | undef |
| Lungs | 0.0050 | 0.0071 | 0.7023 | 1.4239 |
| Stomach- | 0.0097 | 0.0077 | 1.2599 | 0.7937 |
| esophagus | | | | |
| Muscle- | 0.0017 | 0.0300 | 0.0571 | 17.5127 |
| skeleton | | | | |
| Kidneys | 0.0059 | 0.0068 | 0.8683 | 1.1517 |
| Pancreas | 0.0057 | 0.0055 | 1.0285 | 0.9723 |
| Penis | 0.0000 | 0.0267 | 0.0000 | undef |
| Prostate | 0.0024 | 0.0170 | 0.1398 | 7.1516 |
| Uterus | 0.0066 | 0.0000 | undef | 0.0000 |
| Breast- | 0.0036 | | | |
| hyperplasia | | | | |
| Small- | 0.0000 | | | |
| intestine | | | | |
| Prostate- | 0.0119 | | | |
| hyperplasia | | | | |
| Seminal- | 0.0267 | | | |
| vesicles | | | | |
| Sensory organs | 0.0000 | | | |
| White blood | 0.0009 | | | |
| cells | | | | |

| | FETUS |
|---|---|
| | % freq. |
| Development | 0.0000 |
| Gastrointestinal | 0.0031 |
| Brain | 0.0000 |
| Hematopoetic | 0.0000 |
| Heart blood- | 0.0041 |
| vessels | |
| Lungs | 0.0000 |
| Kidneys | 0.0000 |
| Prostate | 0.0000 |
| Sensory organs | 0.0000 |
| | STANDARDIZED/ |
| | SUBTRACTED LIBRARIES |
| | % freq. |
| Breast | 0.0000 |
| Ovary uterus | 0.0068 |
| Endocrine tissue | 0.0245 |
| Fetal | 0.0035 |
| Gastrointestinal | 0.0122 |
| Hematopoetic | 0.0114 |
| Skin muscle | 0.0000 |
| Testicles | 0.0000 |
| Lungs | 0.0164 |
| Nerves | 0.0070 |
| Prostate | 0.0256 |
| Sensory organs | 0.0000 |

Electronic Northern Blot for SEQ. ID. NO: 13

| | NORMAL | TUMOR | RATIOS | |
|---|---|---|---|---|
| | % freq. | % freq. | N/T | T/N |
| Bladder | 0.0000 | 0.0077 | 0.0000 | undef |
| Breast | 0.0200 | 0.0240 | 0.8340 | 1.1991 |
| Ovary | 0.0000 | 0.0000 | undef | undef |
| Endocrine- | 0.0146 | 0.0191 | 0.7655 | 1.3064 |
| tissue | | | | |
| Gastrointestinal | 0.0000 | 0.0000 | undef | undef |
| Brain | 0.0008 | 0.0000 | undef | 0.0000 |
| Hematopoetic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lungs | 0.0012 | 0.0000 | undef | 0.0000 |
| Stomach- | 0.0000 | 0.0000 | undef | undef |
| esophagus | | | | |
| Muscle- | 0.0000 | 0.0000 | undef | undef |
| skeleton | | | | |
| Kidneys | 0.0089 | 0.0000 | undef | 0.0000 |
| Pancreas | 0.0000 | 0.0000 | undef | undef |
| Penis | 0.0000 | 0.0000 | undef | undef |
| Prostate | 0.0000 | 0.0064 | 0.0000 | undef |
| Uterus | 0.0000 | 0.0000 | undef | undef |
| Breast- | 0.0000 | | | |
| hyperplasia | | | | |
| Small- | 0.0000 | | | |
| intestine | | | | |
| Prostate- | 0.0000 | | | |
| hyperplasia | | | | |
| Seminal- | 0.0000 | | | |
| vesicles | | | | |
| Sensory organs | 0.0000 | | | |
| White blood | 0.0017 | | | |
| cells | | | | |

| | FETUS |
|---|---|
| | % freq. |
| Development | 0.0000 |
| Gastrointestinal | 0.0000 |
| Brain | 0.0000 |
| Hematopoetic | 0.0039 |
| Heart blood vessels | 0.0000 |
| Lungs | 0.0000 |
| Kidneys | 0.0124 |
| Prostate | 0.0000 |
| Sensory organs | 0.0419 |
| | STANDARDIZED/ |
| | SUBTRACTED LIBRARIES |
| | % freq. |
| Breast | 0.0068 |
| Ovary uterus | 0.0000 |
| Endocrine tissue | 0.0000 |
| Fetal | 0.0175 |
| Gastrointestinal | 0.0000 |
| Hematopoetic | 0.0000 |
| Skin muscle | 0.0000 |
| Testicles | 0.0000 |
| Lungs | 0.0000 |
| Nerves | 0.0000 |
| Prostate | 0.0000 |
| Sensory organs | 0.0000 |

Electronic Northern Blot for SEQ. ID. NO: 15

| | NORMAL | TUMOR | RATIOS | |
|---|---|---|---|---|
| | % freq. | % freq. | N/T | T/N |
| Bladder | 0.0046 | 0.0128 | 0.3637 | 2.7495 |
| Breast | 0.0027 | 0.0087 | 0.3058 | 3.2702 |
| Ovary | 0.0000 | 0.0052 | 0.0000 | undef |
| Endocrine- | 0.0073 | 0.0245 | 0.2977 | 3.3593 |
| tissue | | | | |
| Gastrointestinal | 0.0019 | 0.0048 | 0.4071 | 2.4562 |
| Brain | 0.0127 | 0.0208 | 0.6111 | 1.6363 |
| Hematopoetic | 0.0098 | 0.0000 | undef | 0.0000 |
| Skin | 0.0000 | 0.0000 | undef | undef |
| Hepatic | 0.0000 | 0.0129 | 0.0000 | undef |
| Heart | 0.0127 | 0.0000 | undef | 0.0000 |
| Testicles | 0.0061 | 0.0234 | 0.2612 | 3.8288 |
| Lungs | 0.0112 | 0.0047 | 2.3702 | 0.4219 |
| Stomach- | 0.0000 | 0.0077 | 0.0000 | undef |
| esophagus | | | | |
| Muscle- | 0.0051 | 0.0000 | undef | 0.0000 |
| skeleton | | | | |
| Kidneys | 0.0238 | 0.0068 | 3.4733 | 0.2879 |
| Pancreas | 0.0095 | 0.0055 | 1.7142 | 0.5834 |
| Penis | 0.0030 | 0.0000 | undef | 0.0000 |
| Prostate | 0.0071 | 0.0277 | 0.2581 | 3.8738 |
| Uterus | 0.0017 | 0.0071 | 0.2321 | 4.3088 |
| Breast- | 0.0145 | | | |
| hyperplasia | | | | |
| Small- | 0.0031 | | | |
| intestine | | | | |
| Prostate- | 0.0178 | | | |
| hyperplasia | | | | |
| Seminal- | 0.0356 | | | |
| vesicles | | | | |
| Sensory organs | 0.0000 | | | |
| White blood | 0.0017 | | | |
| cells | | | | |

| | FETUS |
|---|---|
| | % freq. |
| Development | 0.0000 |
| Gastrointestinal | 0.0000 |
| Brain | 0.0063 |
| Hematopoetic | 0.0079 |
| Heart blood vessels | 0.0041 |
| Lungs | 0.0111 |
| Kidneys | 0.0185 |
| Prostate | 0.0000 |
| Sensory organs | 0.0000 |
| | STANDARDIZED/ |
| | SUBTRACTED LIBRARIES |
| | % freq. |
| Breast | 0.0068 |
| Ovary uterus | 0.0205 |
| Endocrine tissue | 0.0490 |
| Fetal | 0.0017 |
| Gastrointestinal | 0.0000 |
| Hematopoetic | 0.0000 |
| Skin muscle | 0.0130 |
| Testicles | 0.0156 |
| Lungs | 0.0000 |
| Nerves | 0.0020 |
| Prostate | 0.0128 |
| Sensory organs | 0.0000 |

Electronic Northern Blot for SEQ. ID. NO: 17

| | NORMAL | TUMOR | RATIOS | |
|---|---|---|---|---|
| | % freq. | % freq. | N/T | T/N |
| Bladder | 0.0000 | 0.0000 | undef | undef |
| Breast | 0.0000 | 0.0000 | undef | undef |
| Ovary | 0.0000 | 0.0000 | undef | undef |
| Endocrine- | 0.0000 | 0.0000 | undef | undef |
| tissue | | | | |
| Gastrointestinal | 0.0000 | 0.0000 | undef | undef |
| Brain | 0.0000 | 0.0011 | 0.0000 | undef |
| Hematopoetic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lungs | 0.0000 | 0.0000 | undef | undef |
| Stomach- | 0.0000 | 0.0000 | undef | undef |
| esophagus | | | | |
| Muscle- | 0.0000 | 0.0000 | undef | undef |
| skeleton | | | | |
| Kidneys | 0.0030 | 0.0000 | undef | 0.0000 |
| Pancreas | 0.0000 | 0.0000 | undef | undef |
| Penis | 0.0000 | 0.0000 | undef | undef |
| Prostate | 0.0000 | 0.0064 | 0.0000 | undef |
| Uterus | 0.0000 | 0.0000 | undef | undef |
| Breast- | 0.0000 | | | |
| hyperplasia | | | | |
| Small- | 0.0000 | | | |
| intestine | | | | |
| Prostate- | 0.0000 | | | |
| hyperplasia | | | | |
| Seminal- | 0.0000 | | | |
| vesicles | | | | |
| Sensory organs | 0.0000 | | | |
| White blood | 0.0017 | | | |
| cells | | | | |

| | FETUS |
|---|---|
| | % freq. |
| Development | 0.0000 |
| Gastrointestinal | 0.0000 |
| Brain | 0.0000 |
| Hematopoetic | 0.0079 |
| Heart blood vessels | 0.0000 |
| Lungs | 0.0000 |
| Kidneys | 0.0000 |
| Prostate | 0.0000 |
| Sensory organs | 0.0000 |
| | STANDARDIZED/ |
| | SUBTRACTED LIBRARIES |
| | % freq. |
| Breast | 0.0000 |
| Ovary uterus | 0.0023 |
| Endocrine tissue | 0.0000 |
| Fetal | 0.0006 |
| Gastrointestinal | 0.0122 |
| Hematopoetic | 0.0057 |
| Skin muscle | 0.0032 |
| Testicles | 0.0000 |
| Lungs | 0.0000 |
| Nerves | 0.0040 |
| Prostate | 0.0000 |
| Sensory organs | 0.0077 |

Electronic Northern Blot for SEQ. ID. NO: 18

| | NORMAL | TUMOR | RATIOS | |
|---|---|---|---|---|
| | % freq. | % freq. | N/T | T/N |
| Bladder | 0.0000 | 0.0000 | undef | undef |
| Breast | 0.0000 | 0.0000 | undef | undef |
| Ovary | 0.0000 | 0.0000 | undef | undef |
| Endocrine- | 0.0000 | 0.0000 | undef | undef |
| tissue | | | | |
| Gastrointestinal | 0.0000 | 0.0000 | undef | undef |
| Brain | 0.0000 | 0.0000 | undef | undef |
| Hematopoetic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0183 | 0.0000 | undef | 0.0000 |
| Lungs | 0.0000 | 0.0000 | undef | undef |
| Stomach- | 0.0000 | 0.0000 | undef | undef |
| esophagus | | | | |
| Muscle- | 0.0000 | 0.0000 | undef | undef |
| skeleton | | | | |
| Kidneys | 0.0000 | 0.0000 | undef | undef |
| Pancreas | 0.0000 | 0.0000 | undef | undef |
| Penis | 0.0000 | 0.0000 | undef | undef |
| Prostate | 0.0000 | 0.0085 | 0.0000 | undef |
| Uterus | 0.0000 | 0.0000 | undef | undef |
| Breast- | 0.0000 | | | |
| hyperplasia | | | | |
| Small- | 0.0000 | | | |
| intestine | | | | |
| Prostate- | 0.0000 | | | |
| hyperplasia | | | | |
| Seminal- | 0.0000 | | | |
| vesicles | | | | |
| Sensory organs | 0.0000 | | | |
| White blood | 0.0000 | | | |
| cells | | | | |

| | FETUS |
|---|---|
| | % freq. |
| Development | 0.0000 |
| Gastrointestinal | 0.0000 |
| Brain | 0.0000 |
| Hematopoetic | 0.0000 |
| Heart blood vessels | 0.0000 |
| Lungs | 0.0000 |
| Kidneys | 0.0000 |
| Prostate | 0.0000 |
| Sensory organs | 0.0000 |
| | STANDARDIZED/ |
| | SUBTRACTED LIBRARIES |
| | % freq. |
| Breast | 0.0000 |
| Ovary uterus | 0.0000 |
| Endocrine tissue | 0.0000 |
| Fetal | 0.0000 |
| Gastrointestinal | 0.0000 |
| Hematopoetic | 0.0000 |
| Skin muscle | 0.0000 |
| Testicles | 0.0156 |
| Lungs | 0.0000 |
| Nerves | 0.0000 |
| Prostate | 0.0000 |
| Sensory organs | 0.0000 |

Electronic Northern Blot for SEQ. ID. NO: 19

| | NORMAL | TUMOR | RATIOS | |
|---|---|---|---|---|
| | % freq. | % freq. | N/T | T/N |
| Bladder | 0.0000 | 0.0000 | undef | undef |
| Breast | 0.0000 | 0.0022 | 0.0000 | undef |
| Ovary | 0.0000 | 0.0000 | undef | undef |
| Endocrine- | 0.0000 | 0.0000 | undef | undef |
| tissue | | | | |
| Gastrointestinal | 0.0000 | 0.0000 | undef | undef |
| Brain | 0.0008 | 0.0000 | undef | 0.0000 |
| Hematopoetic | 0.0028 | 0.0000 | undef | 0.0000 |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lungs | 0.0000 | 0.0000 | undef | undef |
| Stomach- | 0.0000 | 0.0000 | undef | undef |
| esophagus | | | | |
| Muscle- | 0.0000 | 0.0000 | undef | undef |
| skeleton | | | | |
| Kidneys | 0.0000 | 0.0000 | undef | undef |
| Pancreas | 0.0000 | 0.0000 | undef | undef |
| Penis | 0.0000 | 0.0000 | undef | undef |
| Prostate | 0.0000 | 0.0064 | 0.0000 | undef |
| Uterus | 0.0000 | 0.0000 | undef | undef |
| Breast- | 0.0000 | | | |
| hyperplasia | | | | |
| Small- | 0.0000 | | | |
| intestine | | | | |
| Prostate- | 0.0000 | | | |
| hyperplasia | | | | |
| Seminal- | 0.0000 | | | |
| vesicles | | | | |
| Sensory organs | 0.0000 | | | |
| White blood | 0.0000 | | | |
| cells | | | | |

| | FETUS |
|---|---|
| | % freq. |
| Development | 0.0000 |
| Gastrointestinal | 0.0000 |
| Brain | 0.0000 |
| Hematopoetic | 0.0000 |
| Heart blood vessels | 0.0000 |
| Lungs | 0.0000 |
| Kidneys | 0.0000 |
| Prostate | 0.0000 |
| Sensory organs | 0.0000 |
| | STANDARDIZED/ |
| | SUBTRACTED LIBRARIES |
| | % freq. |
| Breast | 0.0000 |
| Ovary uterus | 0.0000 |
| Endocrine tissue | 0.0000 |
| Fetal | 0.0000 |
| Gastrointestinal | 0.0000 |
| Hematopoetic | 0.0000 |
| Skin muscle | 0.0000 |
| Testicles | 0.0000 |
| Lungs | 0.0000 |
| Nerves | 0.0000 |
| Prostate | 0.0000 |
| Sensory organs | 0.0000 |

Electronic Northern Blot for SEQ. ID. NO: 20

| | NORMAL | TUMOR | RATIOS | |
|---|---|---|---|---|
| | % freq. | % freq. | N/T | T/N |
| Bladder | 0.0046 | 0.0102 | 0.4546 | 2.1996 |
| Breast | 0.0000 | 0.0000 | undef | undef |
| Ovary | 0.0091 | 0.0000 | undef | 0.0000 |
| Endocrine- | 0.0036 | 0.0054 | 0.6698 | 1.4930 |
| tissue | | | | |
| Gastrointestinal | 0.0000 | 0.0048 | 0.0000 | undef |
| Brain | 0.0017 | 0.0044 | 0.3871 | 2.5836 |
| Hematopoetic | 0.0070 | 0.0000 | undef | 0.0000 |
| Skin | 0.0000 | 0.0000 | undef | undef |
| Hepatic | 0.0000 | 0.0065 | 0.0000 | undef |
| Heart | 0.0011 | 0.0000 | undef | 0.0000 |
| Testicles | 0.0061 | 0.0000 | undef | 0.0000 |
| Lungs | 0.0050 | 0.0024 | 2.1069 | 0.4746 |
| Stomach- | 0.0000 | 0.0000 | undef | undef |
| esophagus | | | | |
| Muscle- | 0.0000 | 0.0000 | undef | undef |
| skeleton | | | | |
| Kidneys | 0.0089 | 0.0000 | undef | 0.0000 |
| Pancreas | 0.0000 | 0.0000 | undef | undef |
| Penis | 0.0000 | 0.0000 | undef | undef |
| Prostate | 0.0000 | 0.0043 | 0.0000 | undef |
| Uterus | 0.0017 | 0.0000 | undef | 0.0000 |
| Breast- | 0.0073 | | | |
| hyperplasia | | | | |
| Small- | 0.0031 | | | |
| intestine | | | | |
| Prostate- | 0.0030 | | | |
| hyperplasia | | | | |
| Seminal- | 0.0000 | | | |
| vesicles | | | | |
| Sensory organs | 0.0000 | | | |
| White blood | 0.0035 | | | |
| cells | | | | |

| | FETUS |
|---|---|
| | % freq. |
| Development | 0.0000 |
| Gastrointestinal | 0.0062 |
| Brain | 0.0125 |
| Hematopoetic | 0.0039 |
| Heart blood vessels | 0.0000 |
| Lungs | 0.0037 |
| Kidneys | 0.0124 |
| Prostate | 0.0000 |
| Sensory organs | 0.0000 |
| | STANDARDIZED/ |
| | SUBTRACTED LIBRARIES |
| | % freq. |
| Breast | 0.0204 |
| Ovary uterus | 0.0068 |
| Endocrine tissue | 0.0000 |
| Fetal | 0.0000 |
| Gastrointestinal | 0.0000 |
| Hematopoetic | 0.0000 |
| Skin muscle | 0.0000 |
| Testicles | 0.0000 |
| Lungs | 0.0000 |
| Nerves | 0.0040 |
| Prostate | 0.0192 |
| Sensory organs | 0.0000 |

Electronic Northern Blot for SEQ. ID. NO: 21

| | NORMAL | TUMOR | RATIOS | |
|---|---|---|---|---|
| | % freq. | % freq. | N/T | T/N |
| Bladder | 0.0000 | 0.0000 | undef | undef |
| Breast | 0.0000 | 0.0000 | undef | undef |
| Ovary | 0.0000 | 0.0000 | undef | undef |
| Endocrine- | 0.0000 | 0.0000 | undef | undef |
| tissue | | | | |
| Gastrointestinal | 0.0000 | 0.0000 | undef | undef |
| Brain | 0.0000 | 0.0000 | undef | undef |
| Hematopoetic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lungs | 0.0000 | 0.0000 | undef | undef |
| Stomach- | 0.0000 | 0.0000 | undef | undef |
| esophagus | | | | |
| Muscle- | 0.0000 | 0.0000 | undef | undef |
| skeleton | | | | |
| Kidneys | 0.0000 | 0.0000 | undef | undef |
| Pancreas | 0.0000 | 0.0000 | undef | undef |
| Penis | 0.0000 | 0.0000 | undef | undef |
| Prostate | 0.0000 | 0.0043 | 0.0000 | undef |
| Uterus | 0.0000 | 0.0000 | undef | undef |
| Breast- | 0.0000 | | | |
| hyperplasia | | | | |
| Small- | 0.0000 | | | |
| intestine | | | | |
| Prostate- | 0.0000 | | | |
| hyperplasia | | | | |
| Seminal- | 0.0000 | | | |
| vesicles | | | | |
| Sensory organs | 0.0000 | | | |
| White blood | 0.0009 | | | |
| cells | | | | |

| | FETUS |
|---|---|
| | % freq. |
| Development | 0.0000 |
| Gastrointestinal | 0.0000 |
| Brain | 0.0000 |
| Hematopoetic | 0.0000 |
| Heart blood vessels | 0.0000 |
| Lungs | 0.0000 |
| Kidneys | 0.0000 |
| Prostate | 0.0000 |
| Sensory organs | 0.0000 |
| | STANDARDIZED/ |
| | SUBTRACTED LIBRARIES |
| | % freq. |
| Breast | 0.0000 |
| Ovary uterus | 0.0000 |
| Endocrine tissue | 0.0000 |
| Fetal | 0.0000 |
| Gastrointestinal | 0.0000 |
| Hematopoetic | 0.0000 |
| Skin muscle | 0.0000 |
| Testicles | 0.0000 |
| Lungs | 0.0000 |
| Nerves | 0.0000 |
| Prostate | 0.0000 |
| Sensory organs | 0.0000 |

Electronic Northern Blot for SEQ. ID. NO: 22

| | NORMAL | TUMOR | RATIOS | |
|---|---|---|---|---|
| | % freq. | % freq. | N/T | T/N |
| Bladder | 0.0000 | 0.0000 | undef | undef |
| Breast | 0.0000 | 0.0000 | undef | undef |
| Ovary | 0.0000 | 0.0000 | undef | undef |
| Endocrine- | 0.0000 | 0.0000 | undef | undef |
| tissue | | | | |
| Gastrointestinal | 0.0000 | 0.0000 | undef | undef |
| Brain | 0.0000 | 0.0011 | 0.0000 | undef |
| Hematopoetic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lungs | 0.0000 | 0.0000 | undef | undef |
| Stomach- | 0.0000 | 0.0000 | undef | undef |
| esophagus | | | | |
| Muscle- | 0.0000 | 0.0000 | undef | undef |
| skeleton | | | | |
| Kidneys | 0.0000 | 0.0000 | undef | undef |
| Pancreas | 0.0000 | 0.0000 | undef | undef |
| Penis | 0.0000 | 0.0000 | undef | undef |
| Prostate | 0.0000 | 0.0064 | 0.0000 | undef |
| Uterus | 0.0000 | 0.0000 | undef | undef |
| Breast- | 0.0000 | | | |
| hyperplasia | | | | |
| Small- | 0.0000 | | | |
| intestine | | | | |
| Prostate- | 0.0000 | | | |
| hyperplasia | | | | |
| Seminal- | 0.0000 | | | |
| vesicles | | | | |
| Sensory organs | 0.0000 | | | |
| White blood | 0.0009 | | | |
| cells | | | | |

| | FETUS |
|---|---|
| | % freq. |
| Development | 0.0000 |
| Gastrointestinal | 0.0000 |
| Brain | 0.0000 |
| Hematopoetic | 0.0000 |
| Heart blood vessels | 0.0000 |
| Lungs | 0.0000 |
| Kidneys | 0.0000 |
| Prostate | 0.0000 |
| Sensory organs | 0.0000 |
| | STANDARDIZED/ |
| | SUBTRACTED LIBRARIES |
| | % freq. |
| Breast | 0.0000 |
| Ovary uterus | 0.0000 |
| Endocrine tissue | 0.0000 |
| Fetal | 0.0017 |
| Gastrointestinal | 0.0000 |
| Hematopoetic | 0.0057 |
| Skin muscle | 0.0032 |
| Testicles | 0.0000 |
| Lungs | 0.0000 |
| Nerves | 0.0000 |
| Prostate | 0.0000 |
| Sensory organs | 0.0000 |

Electronic Northern Blot for SEQ. ID. NO: 23

| | NORMAL | TUMOR | RATIOS | |
|---|---|---|---|---|
| | % freq. | % freq. | N/T | T/N |
| Bladder | 0.0000 | 0.0000 | undef | undef |
| Breast | 0.0000 | 0.0000 | undef | undef |
| Ovary | 0.0000 | 0.0000 | undef | undef |
| Endocrine- | 0.0000 | 0.0000 | undef | undef |
| tissue | | | | |
| Gastrointestinal | 0.0000 | 0.0000 | undef | undef |
| Brain | 0.0000 | 0.0000 | undef | undef |
| Hematopoetic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lungs | 0.0000 | 0.0000 | undef | undef |
| Stomach- | 0.0000 | 0.0000 | undef | undef |
| esophagus | | | | |
| Muscle- | 0.0000 | 0.0000 | undef | undef |
| skeleton | | | | |
| Kidneys | 0.0000 | 0.0000 | undef | undef |
| Pancreas | 0.0000 | 0.0000 | undef | undef |
| Penis | 0.0000 | 0.0000 | undef | undef |
| Prostate | 0.0000 | 0.0149 | 0.0000 | undef |
| Uterus | 0.0000 | 0.0000 | undef | undef |
| Breast- | 0.0000 | | | |
| hyperplasia | | | | |
| Small- | 0.0000 | | | |
| intestine | | | | |
| Prostate- | 0.0059 | | | |
| hyperplasia | | | | |
| Seminal | 0.0000 | | | |
| vesicles | | | | |
| Sensory organs | 0.0000 | | | |
| White blood | 0.0000 | | | |
| cells | | | | |

| | FETUS |
|---|---|
| | % freq. |
| Development | 0.0000 |
| Gastrointestinal | 0.0000 |
| Brain | 0.0000 |
| Hematopoetic | 0.0000 |
| Heart blood vessels | 0.0000 |
| Lungs | 0.0000 |
| Kidneys | 0.0000 |
| Prostate | 0.0000 |
| Sensory organs | 0.0000 |
| | STANDARDIZED/ |
| | SUBTRACTED LIBRARIES |
| | % freq. |
| Breast | 0.0000 |
| Ovary uterus | 0.0000 |
| Endocrine tissue | 0.0000 |
| Fetal | 0.0000 |
| Gastrointestinal | 0.0000 |
| Hematopoetic | 0.0000 |
| Skin muscle | 0.0000 |
| Testicles | 0.0000 |
| Lungs | 0.0000 |
| Nerves | 0.0000 |
| Prostate | 0.0000 |
| Sensory organs | 0.0000 |

Electronic Northern Blot for SEQ. ID. NO: 24

| | NORMAL | TUMOR | RATIOS | |
|---|---|---|---|---|
| | % freq. | % freq. | N/T | T/N |
| Bladder | 0.0000 | 0.0000 | undef | undef |
| Breast | 0.0040 | 0.0000 | undef | 0.0000 |
| Ovary | 0.0030 | 0.0052 | 0.5843 | 1.7114 |
| Endocrine- | 0.0000 | 0.0000 | undef | undef |
| tissue | | | | |
| Gastrointestinal | 0.0019 | 0.0000 | undef | 0.0000 |
| Brain | 0.0000 | 0.0011 | 0.0000 | undef |
| Hematopoetic | 0.0000 | 0.0000 | undef | undef |
| Skin | 0.0000 | 0.0000 | undef | undef |
| Hepatic | 0.0000 | 0.0065 | 0.0000 | undef |
| Heart | 0.0011 | 0.0000 | undef | 0.0000 |
| Testicles | 0.0122 | 0.0000 | undef | 0.0000 |
| Lungs | 0.0025 | 0.0000 | undef | 0.0000 |
| Stomach- | 0.0000 | 0.0077 | 0.0000 | undef |
| esophagus | | | | |
| Muscle- | 0.0017 | 0.0000 | undef | 0.0000 |
| skeleton | | | | |
| Kidneys | 0.0149 | 0.0000 | undef | 0.0000 |
| Pancreas | 0.0019 | 0.0000 | undef | 0.0000 |
| Penis | 0.0000 | 0.0000 | undef | undef |
| Prostate | 0.0048 | 0.0106 | 0.4475 | 2.2349 |
| Uterus | 0.0017 | 0.0071 | 0.2321 | 4.3088 |
| Breast- | 0.0036 | | | |
| hyperplasia | | | | |
| Small- | 0.0031 | | | |
| intestine | | | | |
| Prostate- | 0.0000 | | | |
| hyperplasia | | | | |
| Seminal- | 0.0000 | | | |
| vesicles | | | | |
| Sensory organs | 0.0000 | | | |
| White blood | 0.0017 | | | |
| cells | | | | |

| | FETUS |
|---|---|
| | % freq. |
| Development | 0.0154 |
| Gastrointestinal | 0.0031 |
| Brain | 0.0000 |
| Hematopoetic | 0.0039 |
| Heart blood vessels | 0.0000 |
| Lungs | 0.0037 |
| Kidneys | 0.0062 |
| Prostate | 0.0000 |
| Sensory organs | 0.0000 |
| | STANDARDIZED/ |
| | SUBTRACTED LIBRARIES |
| | % freq. |
| Breast | 0.0068 |
| Ovary uterus | 0.0046 |
| Endocrine tissue | 0.0000 |
| Fetal | 0.0012 |
| Gastrointestinal | 0.0000 |
| Hematopoetic | 0.0057 |
| Skin muscle | 0.0032 |
| Testicles | 0.0078 |
| Lungs | 0.0000 |
| Nerves | 0.0000 |
| Prostate | 0.0128 |
| Sensory organs | 0.0000 |

Electronic Northern Blot for SEQ. ID. NO: 26

| | NORMAL | TUMOR | RATIOS | |
|---|---|---|---|---|
| | % freq. | % freq. | N/T | T/N |
| Bladder | 0.0000 | 0.0000 | undef | undef |
| Breast | 0.0000 | 0.0000 | undef | undef |
| Ovary | 0.0000 | 0.0000 | undef | undef |
| Endocrine- | 0.0000 | 0.0000 | undef | undef |
| tissue | | | | |
| Gastrointestinal | 0.0000 | 0.0000 | undef | undef |
| Brain | 0.0000 | 0.0000 | undef | undef |
| Hematopoetic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lungs | 0.0000 | 0.0000 | undef | undef |
| Stomach- | 0.0000 | 0.0000 | undef | undef |
| esophagus | | | | |
| Muscle- | 0.0000 | 0.0000 | undef | undef |
| skeleton | | | | |
| Kidneys | 0.0000 | 0.0000 | undef | undef |
| Pancreas | 0.0000 | 0.0000 | undef | undef |
| Penis | 0.0000 | 0.0000 | undef | undef |
| Prostate | 0.0000 | 0.0064 | 0.0000 | undef |
| Uterus | 0.0000 | 0.0000 | undef | undef |
| Breast- | 0.0000 | | | |
| hyperplasia | | | | |
| Small- | 0.0000 | | | |
| intestine | | | | |
| Prostate- | 0.0000 | | | |
| hyperplasia | | | | |
| Seminal- | 0.0000 | | | |
| vesicles | | | | |
| Sensory organs | 0.0000 | | | |
| White blood | 0.0000 | | | |
| cells | | | | |

| | FETUS |
|---|---|
| | % freq. |
| Development | 0.0000 |
| Gastrointestinal | 0.0000 |
| Brain | 0.0000 |
| Hematopoetic | 0.0000 |
| Heart blood vessels | 0.0000 |
| Lungs | 0.0000 |
| Kidneys | 0.0000 |
| Prostate | 0.0000 |
| Sensory organs | 0.0000 |
| | STANDARDIZED/ |
| | SUBTRACTED LIBRARIES |
| | % freq. |
| Breast | 0.0000 |
| Ovary uterus | 0.0000 |
| Endocrine tissue | 0.0000 |
| Fetal | 0.0000 |
| Gastrointestinal | 0.0000 |
| Hematopoetic | 0.0000 |
| Skin muscle | 0.0000 |
| Testicles | 0.0000 |
| Lungs | 0.0000 |
| Nerves | 0.0000 |
| Prostate | 0.0000 |
| Sensory organs | 0.0000 |

Electronic Northern Blot for SEQ. ID. NO: 27

| | NORMAL | TUMOR | RATIOS | |
|---|---|---|---|---|
| | % freq. | % freq. | N/T | T/N |
| Bladder | 0.0000 | 0.0026 | 0.0000 | undef |
| Breast | 0.0000 | 0.0000 | undef | undef |
| Ovary | 0.0000 | 0.0000 | undef | undef |
| Endocrine- | 0.0000 | 0.0000 | undef | undef |
| tissue | | | | |
| Gastrointestinal | 0.0000 | 0.0000 | undef | undef |
| Brain | 0.0000 | 0.0000 | undef | undef |
| Hematopoetic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lungs | 0.0037 | 0.0000 | undef | 0.0000 |
| Stomach- | 0.0000 | 0.0000 | undef | undef |
| esophagus | | | | |
| Muscle- | 0.0000 | 0.0000 | undef | undef |
| skeleton | | | | |
| Kidneys | 0.0000 | 0.0068 | 0.0000 | undef |
| Pancreas | 0.0000 | 0.0000 | undef | undef |
| Penis | 0.0000 | 0.0000 | undef | undef |
| Prostate | 0.0000 | 0.0064 | 0.0000 | undef |
| Uterus | 0.0017 | 0.0000 | undef | 0.0000 |
| Breast- | 0.0000 | | | |
| hyperplasia | | | | |
| Small- | 0.0000 | | | |
| intestine | | | | |
| Prostate- | 0.0059 | | | |
| hyperplasia | | | | |
| Seminal- | 0.0000 | | | |
| vesicles | | | | |
| Sensory organs | 0.0000 | | | |
| White blood | 0.0000 | | | |
| cells | | | | |

| | FETUS |
|---|---|
| | % freq. |
| Development | 0.0000 |
| Gastrointestinal | 0.0000 |
| Brain | 0.0000 |
| Hematopoetic | 0.0000 |
| Heart blood vessels | 0.0000 |
| Lungs | 0.0000 |
| Kidneys | 0.0000 |
| Prostate | 0.0000 |
| Sensory organs | 0.0000 |
| | STANDARDIZED/ |
| | SUBTRACTED LIBRARIES |
| | % freq. |
| Breast | 0.0000 |
| Ovary uterus | 0.0000 |
| Endocrine tissue | 0.0000 |
| Fetal | 0.0000 |
| Gastrointestinal | 0.0000 |
| Hematopoetic | 0.0000 |
| Skin muscle | 0.0000 |
| Testicles | 0.0000 |
| Lungs | 0.0000 |
| Nerves | 0.0000 |
| Prostate | 0.0192 |
| Sensory organs | 0.0000 |

Electronic Northern Blot for SEQ. ID. NO: 29

| | NORMAL | TUMOR | RATIOS | |
|---|---|---|---|---|
| | % freq. | % freq. | N/T | T/N |
| Bladder | 0.0000 | 0.0000 | undef | undef |
| Breast | 0.0000 | 0.0000 | undef | undef |
| Ovary | 0.0000 | 0.0000 | undef | undef |
| Endocrine- | 0.0000 | 0.0027 | 0.0000 | undef |
| tissue | | | | |
| Gastrointestinal | 0.0000 | 0.0000 | undef | undef |
| Brain | 0.0000 | 0.0000 | undef | undef |
| Hematopoetic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lungs | 0.0012 | 0.0000 | undef | 0.0000 |
| Stomach- | 0.0000 | 0.0000 | undef | undef |
| esophagus | | | | |
| Muscle- | 0.0000 | 0.0000 | undef | undef |
| skeleton | | | | |
| Kidneys | 0.0000 | 0.0000 | undef | undef |
| Pancreas | 0.0000 | 0.0000 | undef | undef |
| Penis | 0.0000 | 0.0000 | undef | undef |
| Prostate | 0.0000 | 0.0106 | 0.0000 | undef |
| Uterus | 0.0000 | 0.0000 | undef | undef |
| Breast- | 0.0000 | | | |
| hyperplasia | | | | |
| Small- | 0.0000 | | | |
| intestine | | | | |
| Prostate | 0.0000 | | | |
| hyperplasia | | | | |
| Seminal- | 0.0000 | | | |
| vesicles | | | | |
| Sensory organs | 0.0000 | | | |
| White blood | 0.0000 | | | |
| cells | | | | |

| | FETUS |
|---|---|
| | % freq. |
| Development | 0.0000 |
| Gastrointestinal | 0.0000 |
| Brain | 0.0000 |
| Hematopoetic | 0.0000 |
| Heart blood vessels | 0.0000 |
| Lungs | 0.0000 |
| Kidneys | 0.0000 |
| Prostate | 0.0000 |
| Sensory organs | 0.0000 |
| | STANDARDIZED/ |
| | SUBTRACTED LIBRARIES |
| | % freq. |
| Breast | 0.0000 |
| Ovary uterus | 0.0000 |
| Endocrine tissue | 0.0000 |
| Fetal | 0.0000 |
| Gastrointestinal | 0.0000 |
| Hematopoetic | 0.0000 |
| Skin muscle | 0.0000 |
| Testicles | 0.0000 |
| Lungs | 0.0000 |
| Nerves | 0.0000 |
| Prostate | 0.0000 |
| Sensory organs | 0.0000 |

Electronic Northern Blot for SEQ. ID. NO: 31

| | NORMAL | TUMOR | RATIOS | |
|---|---|---|---|---|
| | % freq. | % freq. | N/T | T/N |
| Bladder | 0.0046 | 0.0000 | undef | 0.0000 |
| Breast | 0.0027 | 0.0044 | 0.6116 | 1.6351 |
| Ovary | 0.0000 | 0.0000 | undef | undef |
| Endocrine- | 0.0018 | 0.0027 | 0.6698 | 1.4930 |
| tissue | | | | |
| Gastrointestinal | 0.0000 | 0.0000 | undef | undef |
| Brain | 0.0042 | 0.0088 | 0.4838 | 2.0669 |
| Hematopoetic | 0.0000 | 0.0000 | undef | undef |
| Skin | 0.0000 | 0.0000 | undef | undef |
| Hepatic | 0.0050 | 0.0000 | undef | 0.0000 |
| Heart | 0.0021 | 0.0137 | 0.1541 | 6.4872 |
| Testicles | 0.0000 | 0.0000 | undef | undef |
| Lungs | 0.0012 | 0.0000 | undef | 0.0000 |
| Stomach- | 0.0000 | 0.0000 | undef | undef |
| esophagus | | | | |
| Muscle- | 0.0000 | 0.0120 | 0.0000 | undef |
| skeleton | | | | |
| Kidneys | 0.0000 | 0.0000 | undef | undef |
| Pancreas | 0.0000 | 0.0166 | 0.0000 | undef |
| Penis | 0.0000 | 0.0000 | undef | undef |
| Prostate | 0.0000 | 0.0128 | 0.0000 | undef |
| Uterus | 0.0017 | 0.0071 | 0.2321 | 4.3088 |
| Breast- | 0.0000 | | | |
| hyperplasia | | | | |
| Small- | 0.0000 | | | |
| intestine | | | | |
| Prostate- | 0.0059 | | | |
| hyperplasia | | | | |
| Seminal- | 0.0000 | | | |
| vesicles | | | | |
| Sensory organs | 0.0000 | | | |
| White blood | 0.0000 | | | |
| cells | | | | |

| | FETUS |
|---|---|
| | % freq. |
| Development | 0.0154 |
| Gastrointestinal | 0.0000 |
| Brain | 0.0000 |
| Hematopoetic | 0.0157 |
| Heart blood vessels | 0.0041 |
| Lungs | 0.0000 |
| Kidneys | 0.0000 |
| Prostate | 0.0249 |
| Sensory organs | 0.0000 |
| | STANDARDIZED/ |
| | SUBTRACTED LIBRARIES |
| | % freq. |
| Breast | 0.0068 |
| Ovary uterus | 0.0091 |
| Endocrine tissue | 0.0000 |
| Fetal | 0.0000 |
| Gastrointestinal | 0.0000 |
| Hematopoetic | 0.0000 |
| Skin muscle | 0.0000 |
| Testicles | 0.0000 |
| Lungs | 0.0000 |
| Nerves | 0.0060 |
| Prostate | 0.0000 |
| Sensory organs | 0.0000 |

Electronic Northern Blot for SEQ. ID. NO: 33

| | NORMAL | TUMOR | RATIOS | |
|---|---|---|---|---|
| | % freq. | % freq. | N/T | T/N |
| Bladder | 0.0000 | 0.0026 | 0.0000 | undef |
| Breast | 0.0013 | 0.0000 | undef | 0.0000 |
| Ovary | 0.0030 | 0.0078 | 0.3895 | 2.5671 |
| Endocrine- | 0.0000 | 0.0027 | 0.0000 | undef |
| tissue | | | | |
| Gastrointestinal | 0.0019 | 0.0000 | undef | 0.0000 |
| Brain | 0.0017 | 0.0000 | undef | 0.0000 |
| Hematopoetic | 0.0000 | 0.0000 | undef | undef |
| Skin | 0.0000 | 0.0000 | undef | undef |
| Hepatic | 0.0000 | 0.0000 | undef | undef |
| Heart | 0.0011 | 0.0000 | undef | 0.0000 |
| Testicles | 0.0000 | 0.0234 | 0.0000 | undef |
| Lungs | 0.0000 | 0.0000 | undef | undef |
| Stomach- | 0.0000 | 0.0000 | undef | undef |
| esophagus | | | | |
| Muscle- | 0.0017 | 0.0000 | undef | 0.0000 |
| skeleton | | | | |
| Kidneys | 0.0000 | 0.0068 | 0.0000 | undef |
| Pancreas | 0.0000 | 0.0000 | undef | undef |
| Penis | 0.0030 | 0.0000 | undef | 0.0000 |
| Prostate | 0.0000 | 0.0064 | 0.0000 | undef |
| Uterus | 0.0017 | 0.0000 | undef | 0.0000 |
| Breast- | 0.0000 | | | |
| hyperplasia | | | | |
| Small- | 0.0000 | | | |
| intestine | | | | |
| Prostate | 0.0000 | | | |
| hyperplasia | | | | |
| Seminal | 0.0000 | | | |
| vesicles | | | | |
| Sensory organs | 0.0000 | | | |
| White blood | 0.0009 | | | |
| cells | | | | |

| | FETUS |
|---|---|
| | % freq. |
| Development | 0.0000 |
| Gastrointestinal | 0.0000 |
| Brain | 0.0000 |
| Hematopoetic | 0.0000 |
| Heart blood vessels | 0.0000 |
| Lungs | 0.0000 |
| Kidneys | 0.0000 |
| Prostate | 0.0000 |
| Sensory organs | 0.0000 |
| | STANDARDIZED/ |
| | SUBTRACTED LIBRARIES |
| | % freq. |
| Breast | 0.0000 |
| Ovary uterus | 0.0000 |
| Endocrine tissue | 0.0000 |
| Fetal | 0.0017 |
| Gastrointestinal | 0.0000 |
| Hematopoetic | 0.0057 |
| Skin muscle | 0.0000 |
| Testicles | 0.0000 |
| Lungs | 0.0000 |
| Nerves | 0.0050 |
| Prostate | 0.0000 |
| Sensory organs | 0.0000 |

Electronic Northern Blot for SEQ. ID. NO: 34

| | NORMAL | TUMOR | RATIOS | |
|---|---|---|---|---|
| | % freq. | % freq. | N/T | T/N |
| Bladder | 0.0000 | 0.0077 | 0.0000 | undef |
| Breast | 0.0067 | 0.0065 | 1.0193 | 0.9811 |
| Ovary | 0.0122 | 0.0052 | 2.3372 | 0.4279 |
| Endocrine- | 0.0073 | 0.0000 | undef | 0.0000 |
| tissue | | | | |
| Gastrointestinal | 0.0019 | 0.0048 | 0.4071 | 2.4562 |
| Brain | 0.0102 | 0.0033 | 3.0964 | 0.3230 |
| Hematopoetic | 0.0028 | 0.0000 | undef | 0.0000 |
| Skin | 0.0000 | 0.0000 | undef | undef |
| Hepatic | 0.0050 | 0.0065 | 0.7651 | 1.3069 |
| Heart | 0.0011 | 0.0000 | undef | 0.0000 |
| Testicles | 0.0061 | 0.0000 | undef | 0.0000 |
| Lungs | 0.0025 | 0.0000 | undef | 0.0000 |
| Stomach- | 0.0097 | 0.0153 | 0.6300 | 1.5874 |
| esophagus | | | | |
| Muscle- | 0.0086 | 0.0060 | 1.4275 | 0.7005 |
| skeleton | | | | |
| Kidneys | 0.0030 | 0.0137 | 0.2171 | 4.6066 |
| Pancreas | 0.0000 | 0.0000 | undef | undef |
| Penis | 0.0030 | 0.0000 | undef | 0.0000 |
| Prostate | 0.0000 | 0.0128 | 0.0000 | undef |
| Uterus | 0.0033 | 0.0142 | 0.2321 | 4.3088 |
| Breast- | 0.0000 | | | |
| hyperplasia | | | | |
| Small- | 0.0031 | | | |
| intestine | | | | |
| Prostate- | 0.0059 | | | |
| hyperplasia | | | | |
| Seminal- | 0.0000 | | | |
| Sensory organs | 0.0235 | | | |
| White blood | 0.0017 | | | |
| cells | | | | |

| | FETUS |
|---|---|
| | % freq. |
| Development | 0.0000 |
| Gastrointestinal | 0.0062 |
| Brain | 0.0563 |
| Hematopoetic | 0.0118 |
| Heart blood vessels | 0.0082 |
| Lungs | 0.0111 |
| Kidneys | 0.0124 |
| Prostate | 0.0000 |
| Sensory organs | 0.0000 |
| | STANDARDIZED/ |
| | SUBTRACTED LIBRARIES |
| | % freq. |
| Breast | 0.0544 |
| Ovary uterus | 0.0091 |
| Endocrine tissue | 0.0979 |
| Fetal | 0.0058 |
| Gastrointestinal | 0.0122 |
| Hematopoetic | 0.0000 |
| Skin muscle | 0.0097 |
| Testicles | 0.0000 |
| Lungs | 0.0000 |
| Nerves | 0.0161 |
| Prostate | 0.0064 |
| Sensory organs | 0.0000 |

Electronic Northern Blot for SEQ. ID. NO: 37

| | NORMAL | TUMOR | RATIOS | |
|---|---|---|---|---|
| | % freq. | % freq. | N/T | T/N |
| Bladder | 0.0000 | 0.0000 | undef | undef |
| Breast | 0.0027 | 0.0000 | undef | 0.0000 |
| Ovary | 0.0030 | 0.0000 | undef | 0.0000 |
| Endocrine | 0.0000 | 0.0000 | undef | undef |
| tissue | | | | |
| Gastrointestinal | 0.0000 | 0.0000 | undef | undef |
| Brain | 0.0025 | 0.0011 | 2.3223 | 0.4306 |
| Hematopoetic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0117 | 0.0000 | undef |
| Lungs | 0.0087 | 0.0000 | undef | 0.0000 |
| Stomach- | 0.0000 | 0.0000 | undef | undef |
| esophagus | | | | |
| Muscle- | 0.0000 | 0.0000 | undef | undef |
| skeleton | | | | |
| Kidneys | 0.0000 | 0.0000 | undef | undef |
| Pancreas | 0.0000 | 0.0000 | undef | undef |
| Penis | 0.0000 | 0.0000 | undef | undef |
| Prostate | 0.0024 | 0.0128 | 0.1864 | 5.3637 |
| Uterus | 0.0000 | 0.0000 | undef | undef |
| Breast- | 0.0000 | | | |
| hyperplasia | | | | |
| Small- | 0.0000 | | | |
| intestine | | | | |
| Prostate- | 0.0030 | | | |
| hyperplasia | | | | |
| Seminal- | 0.0000 | | | |
| vesicles | | | | |
| Sensory organs | 0.0000 | | | |
| White blood | 0.0000 | | | |
| cells | | | | |

| | FETUS |
|---|---|
| | % freq. |
| Development | 0.0154 |
| Gastrointestinal | 0.0031 |
| Brain | 0.0063 |
| Hematopoetic | 0.0000 |
| Heart blood vessels | 0.0000 |
| Lungs | 0.0037 |
| Kidneys | 0.0000 |
| Prostate | 0.0000 |
| Sensory organs | 0.0140 |
| | STANDARDIZED/ |
| | SUBTRACTED LIBRARIES |
| | % freq. |
| Breast | 0.0000 |
| Ovary uterus | 0.0000 |
| Endocrine tissue | 0.0000 |
| Fetal | 0.0076 |
| Gastrointestinal | 0.0000 |
| Hematopoetic | 0.0000 |
| Skin muscle | 0.0097 |
| Testicles | 0.0000 |
| Lungs | 0.0082 |
| Nerves | 0.0030 |
| Prostate | 0.0064 |
| Sensory organs | 0.0000 |

Electronic Northern Blot for SEQ. ID. NO: 44

| | NORMAL | TUMOR | RATIOS | |
|---|---|---|---|---|
| | % freq. | % freq. | N/T | T/N |
| Bladder | 0.0000 | 0.0128 | 0.0000 | undef |
| Breast | 0.0013 | 0.0044 | 0.3058 | 3.2702 |
| Ovary | 0.0030 | 0.0052 | 0.5843 | 1.7114 |
| Endocrine | 0.0018 | 0.0054 | 0.3349 | 2.9861 |
| tissue | | | | |
| Gastrointestinal | 0.0000 | 0.0000 | undef | undef |
| Brain | 0.0000 | 0.0033 | 0.0000 | undef |
| Hematopoetic | 0.0000 | 0.0378 | 0.0000 | undef |
| Skin | 0.0050 | 0.1693 | 0.0294 | 34.0525 |
| Hepatic | 0.0000 | 0.0000 | undef | undef |
| Heart | 0.0064 | 0.0000 | undef | 0.0000 |
| Testicles | 0.0061 | 0.0117 | 0.5224 | 1.9144 |
| Lungs | 0.0025 | 0.0000 | undef | 0.0000 |
| Stomach- | 0.0000 | 0.0000 | undef | undef |
| esophagus | | | | |
| Muscle- | 0.0086 | 0.0000 | undef | 0.0000 |
| skeleton | | | | |
| Kidneys | 0.0089 | 0.0000 | undef | 0.0000 |
| Pancreas | 0.0000 | 0.0000 | undef | undef |
| Penis | 0.0030 | 0.0000 | undef | 0.0000 |
| Prostate | 0.0024 | 0.0128 | 0.1864 | 5.3637 |
| Uterus | 0.0017 | 0.0000 | undef | 0.0000 |
| Breast- | 0.0000 | | | |
| hyperplasia | | | | |
| Small- | 0.0031 | | | |
| intestine | | | | |
| Prostate- | 0.0000 | | | |
| hyperplasia | | | | |
| Seminal- | 0.0000 | | | |
| vesicles | | | | |
| Sensory organs | 0.0000 | | | |
| White blood | 0.0052 | | | |
| cells | | | | |

| | FETUS |
|---|---|
| | % freq. |
| Development | 0.0000 |
| Gastrointestinal | 0.0000 |
| Brain | 0.0000 |
| Hematopoetic | 0.0039 |
| Heart blood vessels | 0.0041 |
| Lungs | 0.0037 |
| Kidneys | 0.0000 |
| Prostate | 0.0000 |
| Sensory organs | 0.0000 |
| | STANDARDIZED/ |
| | SUBTRACTED LIBRARIES |
| | % freq. |
| Breast | 0.0000 |
| Ovary uterus | 0.0000 |
| Endocrine tissue | 0.0000 |
| Fetal | 0.0041 |
| Gastrointestinal | 0.0122 |
| Hematopoetic | 0.0114 |
| Skin muscle | 0.0065 |
| Testicles | 0.0000 |
| Lungs | 0.0000 |
| Nerves | 0.0030 |
| Prostate | 0.0064 |
| Sensory organs | 0.0000 |

Electronic Northern Blot for SEQ. ID. NO: 45

| | NORMAL | TUMOR | RATIOS | |
|---|---|---|---|---|
| | % freq. | % freq. | N/T | T/N |
| Bladder | 0.0000 | 0.0179 | 0.0000 | undef |
| Breast | 0.0013 | 0.0044 | 0.3058 | 3.2702 |
| Ovary | 0.0030 | 0.0026 | 1.1686 | 0.8557 |
| Endocrine | 0.0018 | 0.0000 | undef | 0.0000 |
| tissue | | | | |
| Gastrointestinal | 0.0097 | 0.0000 | undef | 0.0000 |
| Brain | 0.0000 | 0.0022 | 0.0000 | undef |
| Hematopoetic | 0.0014 | 0.0000 | undef | 0.0000 |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lungs | 0.0012 | 0.0000 | undef | 0.0000 |
| Stomach- | 0.0000 | 0.0077 | 0.0000 | undef |
| esophagus | | | | |
| Muscle- | 0.0000 | 0.0000 | undef | undef |
| skeleton | | | | |
| Kidneys | 0.0000 | 0.0068 | 0.0000 | undef |
| Pancreas | 0.0000 | 0.0055 | 0.0000 | undef |
| Penis | 0.0000 | 0.0000 | undef | undef |
| Prostate | 0.0024 | 0.0447 | 0.0533 | 18.7729 |
| Uterus | 0.0000 | 0.0071 | 0.0000 | undef |
| Breast- | 0.0109 | | | |
| hyperplasia | | | | |
| Small- | 0.0000 | | | |
| intestine | | | | |
| Prostate- | 0.0030 | | | |
| hyperplasia | | | | |
| Seminal- | 0.0089 | | | |
| vesicles | | | | |
| Sensory organs | 0.0000 | | | |
| White blood | 0.0000 | | | |
| cells | | | | |

| | FETUS |
|---|---|
| | % freq. |
| Development | 0.0000 |
| Gastrointestinal | 0.0000 |
| Brain | 0.0000 |
| Hematopoetic | 0.0000 |
| Heart blood vessels | 0.0000 |
| Lungs | 0.0037 |
| Kidneys | 0.0062 |
| Prostate | 0.0000 |
| Sensory organs | 0.0000 |
| | STANDARDIZED/ |
| | SUBTRACTED LIBRARIES |
| | % freq. |
| Breast | 0.0000 |
| Ovary uterus | 0.0000 |
| Endocrine tissue | 0.0000 |
| Fetal | 0.0000 |
| Gastrointestinal | 0.0000 |
| Hematopoetic | 0.0000 |
| Skin muscle | 0.0000 |
| Testicles | 0.0000 |
| Lungs | 0.0082 |
| Nerves | 0.0010 |
| Prostate | 0.0064 |
| Sensory organs | 0.0000 |

Electronic Northern Blot for SEQ. ID. NO: 46

| | NORMAL | TUMOR | RATIOS | |
|---|---|---|---|---|
| | % freq. | % freq. | N/T | T/N |
| Bladder | 0.0000 | 0.0051 | 0.0000 | undef |
| Breast | 0.0053 | 0.0153 | 0.3495 | 2.8614 |
| Ovary | 0.0091 | 0.0182 | 0.5008 | 1.9967 |
| Endocrine- | 0.0055 | 0.0191 | 0.2870 | 3.4837 |
| tissue | | | | |
| Gastrointestinal | 0.0252 | 0.0238 | 1.0585 | 0.9447 |
| Brain | 0.0068 | 0.0153 | 0.4423 | 2.2607 |
| Hematopoetic | 0.0042 | 0.0000 | undef | 0.0000 |
| Skin | 0.0000 | 0.0000 | undef | undef |
| Hepatic | 0.0099 | 0.0065 | 1.5303 | 0.6535 |
| Heart | 0.0000 | 0.0412 | 0.0000 | undef |
| Testicles | 0.0000 | 0.0000 | undef | undef |
| Lungs | 0.0025 | 0.0095 | 0.2634 | 3.7971 |
| Stomach- | 0.0290 | 0.0000 | undef | 0.0000 |
| esophagus | | | | |
| Muscle- | 0.0103 | 0.0060 | 1.7130 | 0.5838 |
| skeleton | | | | |
| Kidneys | 0.0059 | 0.0068 | 0.8683 | 1.1517 |
| Pancreas | 0.0038 | 0.0221 | 0.1714 | 5.8337 |
| Penis | 0.0060 | 0.0000 | undef | 0.0000 |
| Prostate | 0.0262 | 0.0532 | 0.4922 | 2.0317 |
| Uterus | 0.0033 | 0.0142 | 0.2321 | 4.3088 |
| Breast- | 0.0036 | | | |
| hyperplasia | | | | |
| Small- | 0.0125 | | | |
| intestine | | | | |
| Prostate- | 0.0119 | | | |
| hyperplasia | | | | |
| Seminal- | 0.0000 | | | |
| vesicles | | | | |
| Sensory organs | 0.0000 | | | |
| White blood | 0.0009 | | | |
| cells | | | | |

| | FETUS |
|---|---|
| | % freq. |
| Development | 0.0154 |
| Gastrointestinal | 0.0031 |
| Brain | 0.0125 |
| Hematopoetic | 0.0000 |
| Heart blood vessels | 0.0041 |
| Lungs | 0.0000 |
| Kidneys | 0.0000 |
| Prostate | 0.0000 |
| Sensory organs | 0.0000 |
| | STANDARDIZED/ |
| | SUBTRACTED LIBRARIES |
| | % freq. |
| Breast | 0.0136 |
| Ovary uterus | 0.0137 |
| Endocrine tissue | 0.0000 |
| Fetal | 0.0070 |
| Gastrointestinal | 0.0122 |
| Hematopoetic | 0.0000 |
| Skin muscle | 0.0097 |
| Testicles | 0.0000 |
| Lungs | 0.0082 |
| Nerves | 0.0110 |
| Prostate | 0.0321 |
| Sensory organs | 0.0000 |

Electronic Northern Blot for SEQ. ID. NO: 47

| | NORMAL | TUMOR | RATIOS | |
|---|---|---|---|---|
| | % freq. | % freq. | N/T | T/N |
| Bladder | 0.0046 | 0.0051 | 0.9092 | 1.0998 |
| Breast | 0.0080 | 0.0087 | 0.9174 | 1.0901 |
| Ovary | 0.0061 | 0.0000 | undef | 0.0000 |
| Endocrine- | 0.0073 | 0.0054 | 1.3396 | 0.7465 |
| tissue | | | | |
| Gastrointestinal | 0.0000 | 0.0000 | undef | undef |
| Brain | 0.0051 | 0.0088 | 0.5806 | 1.7224 |
| Hematopoetic | 0.0070 | 0.0000 | undef | 0.0000 |
| Skin | 0.0000 | 0.0000 | undef | undef |
| Hepatic | 0.0000 | 0.0000 | undef | undef |
| Heart | 0.0021 | 0.0000 | undef | 0.0000 |
| Testicles | 0.0000 | 0.0000 | undef | undef |
| Lungs | 0.0062 | 0.0024 | 2.6336 | 0.3797 |
| Stomach- | 0.0000 | 0.0077 | 0.0000 | undef |
| esophagus | | | | |
| Muscle- | 0.0086 | 0.0000 | undef | 0.0000 |
| skeleton | | | | |
| Kidneys | 0.0059 | 0.0068 | 0.8683 | 1.1517 |
| Pancreas | 0.0057 | 0.0000 | undef | 0.0000 |
| Penis | 0.0090 | 0.0000 | undef | 0.0000 |
| Prostate | 0.0048 | 0.0128 | 0.3729 | 2.6818 |
| Uterus | 0.0083 | 0.0071 | 1.1604 | 0.8618 |
| Breast- | 0.0000 | | | |
| hyperplasia | | | | |
| Small- | 0.0125 | | | |
| intestine | | | | |
| Prostate- | 0.0059 | | | |
| hyperplasia | | | | |
| Seminal- | 0.0000 | | | |
| vesicles | | | | |
| Sensory organs | 0.0000 | | | |
| White blood | 0.0061 | | | |
| cells | | | | |

| | FETUS |
|---|---|
| | % freq. |
| Development | 0.0000 |
| Gastrointestinal | 0.0031 |
| Brain | 0.0000 |
| Hematopoetic | 0.0000 |
| Heart blood vessels | 0.0041 |
| Lungs | 0.0037 |
| Kidneys | 0.0185 |
| Prostate | 0.0000 |
| Sensory organs | 0.0000 |
| | STANDARDIZED/ |
| | SUBTRACTED LIBRARIES |
| | % freq. |
| Breast | 0.0000 |
| Ovary uterus | 0.0068 |
| Endocrine tissue | 0.0000 |
| Fetal | 0.0012 |
| Gastrointestinal | 0.0122 |
| Hematopoetic | 0.0000 |
| Skin muscle | 0.0000 |
| Testicles | 0.0000 |
| Lungs | 0.0328 |
| Nerves | 0.0080 |
| Prostate | 0.0064 |
| Sensory organs | 0.0000 |

Electronic Northern Blot for SEQ. ID. NO: 48

| | NORMAL | TUMOR | RATIOS | |
|---|---|---|---|---|
| | % freq. | % freq. | N/T | T/N |
| Bladder | 0.0000 | 0.0153 | 0.0000 | undef |
| Breast | 0.0040 | 0.0044 | 0.9174 | 1.0901 |
| Ovary | 0.0000 | 0.0104 | 0.0000 | undef |
| Endocrine- | 0.0018 | 0.0000 | undef | 0.0000 |
| tissue | | | | |
| Gastrointestinal | 0.0019 | 0.0095 | 0.2036 | 4.9124 |
| Brain | 0.0000 | 0.0055 | 0.0000 | undef |
| Hematopoetic | 0.0098 | 0.0000 | undef | 0.0000 |
| Skin | 0.0050 | 0.0000 | undef | 0.0000 |
| Hepatic | 0.0000 | 0.0194 | 0.0000 | undef |
| Heart | 0.0021 | 0.0000 | undef | 0.0000 |
| Testicles | 0.0000 | 0.0000 | undef | undef |
| Lungs | 0.0012 | 0.0047 | 0.2634 | 3.7971 |
| Stomach- | 0.0097 | 0.0077 | 1.2599 | 0.7937 |
| esophagus | | | | |
| Muscle- | 0.0000 | 0.0000 | undef | undef |
| skeleton | | | | |
| Kidneys | 0.0059 | 0.0068 | 0.8683 | 1.1517 |
| Pancreas | 0.0057 | 0.0000 | undef | 0.0000 |
| Penis | 0.0000 | 0.0267 | 0.0000 | undef |
| Prostate | 0.0048 | 0.0298 | 0.1598 | 6.2576 |
| Uterus | 0.0033 | 0.0071 | 0.4642 | 2.1544 |
| Breast- | 0.0000 | | | |
| hyperplasia | | | | |
| Small- | 0.0031 | | | |
| intestine | | | | |
| Prostate- | 0.0238 | | | |
| hyperplasia | | | | |
| Seminal- | 0.0000 | | | |
| vesicles | | | | |
| Sensory organs | 0.0000 | | | |
| White blood | 0.0026 | | | |
| cells | | | | |

| | FETUS |
|---|---|
| | % freq. |
| Development | 0.0154 |
| Gastrointestinal | 0.0154 |
| Brain | 0.0063 |
| Hematopoetic | 0.0000 |
| Heart blood vessels | 0.0041 |
| Lungs | 0.0111 |
| Kidneys | 0.0000 |
| Prostate | 0.0499 |
| Sensory organs | 0.0000 |
| | STANDARDIZED/ |
| | SUBTRACTED LIBRARIES |
| | % freq. |
| Breast | 0.0000 |
| Ovary uterus | 0.0160 |
| Endocrine tissue | 0.0000 |
| Fetal | 0.0041 |
| Gastrointestinal | 0.0000 |
| Hematopoetic | 0.0000 |
| Skin muscle | 0.0000 |
| Testicles | 0.0000 |
| Lungs | 0.0000 |
| Nerves | 0.0030 |
| Prostate | 0.0128 |
| Sensory organs | 0.0000 |

Electronic Northern Blot for SEQ. ID. NO: 49

| | NORMAL | TUMOR | RATIOS | |
|---|---|---|---|---|
| | % freq. | % freq. | N/T | T/N |
| Bladder | 0.0000 | 0.0026 | 0.0000 | undef |
| Breast | 0.0067 | 0.0065 | 1.0193 | 0.9811 |
| Ovary | 0.0061 | 0.0052 | 1.1686 | 0.8557 |
| Endocrine- | 0.0055 | 0.0027 | 2.0093 | 0.4977 |
| tissue | | | | |
| Gastrointestinal | 0.0019 | 0.0048 | 0.4071 | 2.4562 |
| Brain | 0.0008 | 0.0044 | 0.1935 | 5.1673 |
| Hematopoetic | 0.0042 | 0.0000 | undef | 0.0000 |
| Skin | 0.0050 | 0.0000 | undef | 0.0000 |
| Hepatic | 0.0099 | 0.0000 | undef | 0.0000 |
| Heart | 0.0064 | 0.0275 | 0.2312 | 4.3248 |
| Testicles | 0.0061 | 0.0117 | 0.5224 | 1.9144 |
| Lungs | 0.0087 | 0.0071 | 1.2290 | 0.8137 |
| Stomach- | 0.0097 | 0.0077 | 1.2599 | 0.7937 |
| esophagus | | | | |
| Muscle- | 0.0086 | 0.0060 | 1.4275 | 0.7005 |
| skeleton | | | | |
| Kidneys | 0.0059 | 0.0068 | 0.8683 | 1.1517 |
| Pancreas | 0.0000 | 0.0000 | undef | undef |
| Penis | 0.0000 | 0.0533 | 0.0000 | undef |
| Prostate | 0.0024 | 0.0170 | 0.1398 | 7.1516 |
| Uterus | 0.0066 | 0.0142 | 0.4642 | 2.1544 |
| Breast- | 0.0000 | | | |
| hyperplasia | | | | |
| Small- | 0.0062 | | | |
| intestine | | | | |
| Prostate- | 0.0000 | | | |
| hyperplasia | | | | |
| Seminal- | 0.0000 | | | |
| vesicles | | | | |
| Sensory organs | 0.0118 | | | |
| White blood | 0.0000 | | | |
| cells | | | | |

| | FETUS |
|---|---|
| | % freq. |
| Development | 0.0000 |
| Gastrointestinal | 0.0092 |
| Brain | 0.0000 |
| Hematopoetic | 0.0118 |
| Heart blood vessels | 0.0204 |
| Lungs | 0.0185 |
| Kidneys | 0.0247 |
| Prostate | 0.0249 |
| Sensory organs | 0.0000 |
| | STANDARDIZED/ |
| | SUBTRACTED LIBRARIES |
| | % freq. |
| Breast | 0.0136 |
| Ovary uterus | 0.0023 |
| Endocrine tissue | 0.0000 |
| Fetal | 0.0111 |
| Gastrointestinal | 0.0000 |
| Hematopoetic | 0.0057 |
| Skin muscle | 0.0194 |
| Testicles | 0.0000 |
| Lungs | 0.0328 |
| Nerves | 0.0020 |
| Prostate | 0.0064 |
| Sensory organs | 0.0000 |

Electronic Northern Blot for SEQ. ID. NO: 50

| | NORMAL | TUMOR | RATIOS | |
|---|---|---|---|---|
| | % freq. | % freq. | N/T | T/N |
| Bladder | 0.0000 | 0.0000 | undef | undef |
| Breast | 0.0000 | 0.0000 | undef | undef |
| Ovary | 0.0000 | 0.0000 | undef | undef |
| Endocrine- | 0.0000 | 0.0000 | undef | undef |
| tissue | | | | |
| Gastrointestinal | 0.0000 | 0.0000 | undef | undef |
| Brain | 0.0000 | 0.0000 | undef | undef |
| Hematopoetic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lungs | 0.0000 | 0.0000 | undef | undef |
| Stomach- | 0.0000 | 0.0000 | undef | undef |
| esophagus | | | | |
| Muscle- | 0.0000 | 0.0000 | undef | undef |
| skeleton | | | | |
| Kidneys | 0.0000 | 0.0000 | undef | undef |
| Pancreas | 0.0000 | 0.0000 | undef | undef |
| Penis | 0.0000 | 0.0000 | undef | undef |
| Prostate | 0.0024 | 0.0255 | 0.0932 | 10.7274 |
| Uterus | 0.0000 | 0.0000 | undef | undef |
| Breast- | 0.0000 | | | |
| hyperplasia | | | | |
| Small- | 0.0000 | | | |
| intestine | | | | |
| Prostate- | 0.0030 | | | |
| hyperplasia | | | | |
| Seminal- | 0.0000 | | | |
| vesicles | | | | |
| Sensory organs | 0.0000 | | | |
| White blood | 0.0000 | | | |
| cells | | | | |

| | FETUS |
|---|---|
| | % freq. |
| Development | 0.0000 |
| Gastrointestinal | 0.0000 |
| Brain | 0.0000 |
| Hematopoetic | 0.0000 |
| Heart blood vessels | 0.0000 |
| Lungs | 0.0000 |
| Kidneys | 0.0000 |
| Prostate | 0.0000 |
| Sensory organs | 0.0000 |
| | STANDARDIZED/ |
| | SUBTRACTED LIBRARIES |
| | % freq. |
| Breast | 0.0000 |
| Ovary uterus | 0.0000 |
| Endocrine tissue | 0.0000 |
| Fetal | 0.0000 |
| Gastrointestinal | 0.0000 |
| Hematopoetic | 0.0000 |
| Skin muscle | 0.0000 |
| Testicles | 0.0000 |
| Lungs | 0.0000 |
| Nerves | 0.0000 |
| Prostate | 0.0000 |
| Sensory organs | 0.0000 |

Electronic Northern Blot for SEQ. ID. NO: 51

| | NORMAL | TUMOR | RATIOS | |
|---|---|---|---|---|
| | % freq. | % freq. | N/T | T/N |
| Bladder | 0.0000 | 0.0077 | 0.0000 | undef |
| Breast | 0.0000 | 0.0022 | 0.0000 | undef |
| Ovary | 0.0000 | 0.0000 | undef | undef |
| Endocrine | 0.0000 | 0.0000 | undef | undef |
| tissue | | | | |
| Gastrointestinal | 0.0000 | 0.0000 | undef | undef |
| Brain | 0.0008 | 0.0033 | 0.2580 | 3.8754 |
| Hematopoetic | 0.0000 | 0.0000 | undef | undef |
| Skin | 0.0000 | 0.0000 | undef | undef |
| Hepatic | 0.0000 | 0.0065 | 0.0000 | undef |
| Heart | 0.0000 | 0.0000 | undef | undef |
| Testicles | 0.0061 | 0.0000 | undef | 0.0000 |
| Lungs | 0.0000 | 0.0000 | undef | undef |
| Stomach- | 0.0097 | 0.0000 | undef | 0.0000 |
| esophagus | | | | |
| Muscle- | 0.0000 | 0.0000 | undef | undef |
| skeleton | | | | |
| Kidneys | 0.0000 | 0.0000 | undef | undef |
| Pancreas | 0.0000 | 0.0000 | undef | undef |
| Penis | 0.0000 | 0.0267 | 0.0000 | undef |
| Prostate | 0.0048 | 0.0192 | 0.2486 | 4.0228 |
| Uterus | 0.0017 | 0.0142 | 0.1160 | 8.6176 |
| Breast- | 0.0000 | | | |
| hyperplasia | | | | |
| Small- | 0.0000 | | | |
| intestine | | | | |
| Prostate- | 0.0030 | | | |
| hyperplasia | | | | |
| Seminal- | 0.0000 | | | |
| vesicles | | | | |
| Sensory organs | 0.0000 | | | |
| White blood | 0.0000 | | | |
| cells | | | | |

| | FETUS |
|---|---|
| | % freq. |
| Development | 0.0000 |
| Gastrointestinal | 0.0031 |
| Brain | 0.0000 |
| Hematopoetic | 0.0039 |
| Heart blood vessels | 0.0000 |
| Lungs | 0.0000 |
| Kidneys | 0.0000 |
| Prostate | 0.0000 |
| Sensory organs | 0.0000 |
| | STANDARDIZED/ |
| | SUBTRACTED LIBRARIES |
| | % freq. |
| Breast | 0.0000 |
| Ovary uterus | 0.0000 |
| Endocrine tissue | 0.0000 |
| Fetal | 0.0012 |
| Gastrointestinal | 0.0000 |
| Hematopoetic | 0.0000 |
| Skin muscle | 0.0000 |
| Testicles | 0.0000 |
| Lungs | 0.0082 |
| Nerves | 0.0000 |
| Prostate | 0.0128 |
| Sensory organs | 0.0000 |

Electronic Northern Blot for SEQ. ID. NO: 52

| | NORMAL | TUMOR | RATIOS | |
|---|---|---|---|---|
| | % freq. | % freq. | N/T | T/N |
| Bladder | 0.0046 | 0.0077 | 0.6062 | 1.6497 |
| Breast | 0.0027 | 0.0022 | 1.2232 | 0.8176 |
| Ovary | 0.0030 | 0.0026 | 1.1686 | 0.8557 |
| Endocrine | 0.0000 | 0.0000 | undef | undef |
| tissue | | | | |
| Gastrointestinal | 0.0039 | 0.0000 | undef | 0.0000 |
| Brain | 0.0076 | 0.0022 | 3.4835 | 0.2871 |
| Hematopoetic | 0.0042 | 0.0000 | undef | 0.0000 |
| Skin | 0.0000 | 0.0000 | undef | undef |
| Hepatic | 0.0000 | 0.0065 | 0.0000 | undef |
| Heart | 0.0000 | 0.0000 | undef | undef |
| Testicles | 0.0000 | 0.0000 | undef | undef |
| Lungs | 0.0000 | 0.0071 | 0.0000 | undef |
| Stomach- | 0.0000 | 0.0000 | undef | undef |
| esophagus | | | | |
| Muscle- | 0.0034 | 0.0060 | 0.5710 | 1.7513 |
| skeleton | | | | |
| Kidneys | 0.0030 | 0.0000 | undef | 0.0000 |
| Pancreas | 0.0000 | 0.0055 | 0.0000 | undef |
| Penis | 0.0000 | 0.0000 | undef | undef |
| Prostate | 0.0048 | 0.0149 | 0.3196 | 3.1288 |
| Uterus | 0.0017 | 0.0000 | undef | 0.0000 |
| Breast- | 0.0036 | | | |
| hyperplasia | | | | |
| Small- | 0.0062 | | | |
| intestine | | | | |
| Prostate- | 0.0059 | | | |
| hyperplasia | | | | |
| Seminal- | 0.0000 | | | |
| vesicles | | | | |
| Sensory organs | 0.0000 | | | |
| White blood | 0.0017 | | | |
| cells | | | | |

| | FETUS |
|---|---|
| | % freq. |
| Development | 0.0000 |
| Gastrointestinal | 0.0062 |
| Brain | 0.0000 |
| Hematopoetic | 0.0039 |
| Heart blood vessels | 0.0041 |
| Lungs | 0.0037 |
| Kidneys | 0.0000 |
| Prostate | 0.0000 |
| Sensory organs | 0.0000 |
| | STANDARDIZED/ |
| | SUBTRACTED LIBRARIES |
| | % freq. |
| Breast | 0.0068 |
| Ovary uterus | 0.0023 |
| Endocrine tissue | 0.0000 |
| Fetal | 0.0017 |
| Gastrointestinal | 0.0244 |
| Hematopoetic | 0.0000 |
| Skin muscle | 0.0000 |
| Testicles | 0.0000 |
| Lungs | 0.0000 |
| Nerves | 0.0070 |
| Prostate | 0.0064 |
| Sensory organs | 0.0000 |

Electronic Northern Blot for SEQ. ID. NO: 53

| | NORMAL | TUMOR | RATIOS | |
|---|---|---|---|---|
| | % freq. | % freq. | N/T | T/N |
| Bladder | 0.0093 | 0.0128 | 0.7274 | 1.3748 |
| Breast | 0.0053 | 0.0022 | 2.4463 | 0.4088 |
| Ovary | 0.0030 | 0.0026 | 1.1686 | 0.8557 |
| Endocrine- | 0.0109 | 0.0082 | 1.3396 | 0.7465 |
| tissue | | | | |
| Gastrointestinal | 0.0039 | 0.0048 | 0.8143 | 1.2281 |
| Brain | 0.0042 | 0.0153 | 0.2765 | 3.6171 |
| Hematopoetic | 0.0042 | 0.0000 | undef | 0.0000 |
| Skin | 0.0000 | 0.0000 | undef | undef |
| Hepatic | 0.0050 | 0.0000 | undef | 0.0000 |
| Heart | 0.0064 | 0.0000 | undef | 0.0000 |
| Testicles | 0.0061 | 0.0117 | 0.5224 | 1.9144 |
| Lungs | 0.0075 | 0.0024 | 3.1603 | 0.3164 |
| Stomach- | 0.0193 | 0.0153 | 1.2599 | 0.7937 |
| esophagus | | | | |
| Muscle- | 0.0000 | 0.0000 | undef | undef |
| skeleton | | | | |
| Kidneys | 0.0000 | 0.0137 | 0.0000 | undef |
| Pancreas | 0.0057 | 0.0110 | 0.5143 | 1.9446 |
| Penis | 0.0030 | 0.0000 | undef | 0.0000 |
| Prostate | 0.0048 | 0.0106 | 0.4475 | 2.2349 |
| Uterus | 0.0017 | 0.0000 | undef | 0.0000 |
| Breast- | 0.0145 | | | |
| hyperplasia | | | | |
| Small- | 0.0062 | | | |
| intestine | | | | |
| Prostate- | 0.0000 | | | |
| hyperplasia | | | | |
| Seminal- | 0.0089 | | | |
| Sensory organs | 0.0000 | | | |
| White blood | 0.0044 | | | |
| cells | | | | |

| | FETUS |
|---|---|
| | % freq. |
| Development | 0.0000 |
| Gastrointestinal | 0.0123 |
| Brain | 0.0063 |
| Hematopoetic | 0.0000 |
| Heart blood vessels | 0.0123 |
| Lungs | 0.0037 |
| Kidneys | 0.0000 |
| Prostate | 0.0000 |
| Sensory organs | 0.0000 |
| | STANDARDIZED/ |
| | SUBTRACTED LIBRARIES |
| | % freq. |
| Breast | 0.0136 |
| Ovary uterus | 0.0160 |
| Endocrine tissue | 0.0000 |
| Fetal | 0.0140 |
| Gastrointestinal | 0.0122 |
| Hematopoetic | 0.0057 |
| Skin muscle | 0.0130 |
| Testicles | 0.0156 |
| Lungs | 0.0082 |
| Nerves | 0.0090 |
| Prostate | 0.0128 |
| Sensory organs | 0.0000 |

Electronic Northern Blot for SEQ. ID. NO: 142

| | NORMAL | TUMOR | RATIOS | |
|---|---|---|---|---|
| | % freq. | % freq. | N/T | T/N |
| Bladder | 0.0093 | 0.0026 | 3.6370 | 0.2750 |
| Breast | 0.0067 | 0.0131 | 0.5096 | 1.9621 |
| Ovary | 0.0122 | 0.0130 | 0.9349 | 1.0696 |
| Endocrine- | 0.0036 | 0.0027 | 1.3396 | 0.7465 |
| tissue | | | | |
| Gastrointestinal | 0.0058 | 0.0048 | 1.2214 | 0.8187 |
| Brain | 0.0161 | 0.0044 | 3.6770 | 0.2720 |
| Hematopoetic | 0.0056 | 0.0000 | undef | 0.0000 |
| Skin | 0.0000 | 0.0000 | undef | undef |
| Hepatic | 0.0099 | 0.0065 | 1.5303 | 0.6535 |
| Heart | 0.0117 | 0.0000 | undef | 0.0000 |
| Testicles | 0.0000 | 0.0117 | 0.0000 | undef |
| Lungs | 0.0075 | 0.0189 | 0.3950 | 2.5314 |
| Stomach- | 0.0000 | 0.0000 | undef | undef |
| esophagus | | | | |
| Muscle- | 0.0069 | 0.0060 | 1.1420 | 0.8756 |
| skeleton | | | | |
| Kidneys | 0.0000 | 0.0068 | 0.0000 | undef |
| Pancreas | 0.0057 | 0.0000 | undef | 0.0000 |
| Penis | 0.0000 | 0.0267 | 0.0000 | undef |
| Prostate | 0.0024 | 0.0085 | 0.2797 | 3.5758 |
| Uterus | 0.0033 | 0.0142 | 0.2321 | 4.3088 |
| Breast- | 0.0036 | | | |
| hyperplasia | | | | |
| Small- | 0.0125 | | | |
| intestine | | | | |
| Prostate- | 0.0030 | | | |
| hyperplasia | | | | |
| Seminal- | 0.0000 | | | |
| vesicles | | | | |
| Sensory organs | 0.0000 | | | |
| White blood | 0.0017 | | | |
| cells | | | | |

| | FETUS |
|---|---|
| | % freq. |
| Development | 0.0000 |
| Gastrointestinal | 0.0092 |
| Brain | 0.0125 |
| Hematopoetic | 0.0000 |
| Heart blood vessels | 0.0041 |
| Lungs | 0.0148 |
| Kidneys | 0.0000 |
| Prostate | 0.0000 |
| Sensory organs | 0.0140 |
| | STANDARDIZED/ |
| | SUBTRACTED LIBRARIES |
| | % freq. |
| Breast | 0.0000 |
| Ovary uterus | 0.0023 |
| Endocrine tissue | 0.0000 |
| Fetal | 0.0012 |
| Gastrointestinal | 0.0000 |
| Hematopoetic | 0.0114 |
| Skin muscle | 0.0000 |
| Testicles | 0.0000 |
| Lungs | 0.0410 |
| Nerves | 0.0090 |
| Prostate | 0.0192 |
| Sensory organs | 0.0000 |

Electronic Northern for SEQ. ID: 144

| | NORMAL | TUMOR | RATIOS | |
|---|---|---|---|---|
| | % freq. | % freq. | N/T | T/N |
| B_Lymphoma | 0.0025 | 0.0000 | undef | 0.0000 |
| Bladder | 0.0039 | 0.0141 | 0.2766 | 3.6156 |
| Breast | 0.0062 | 0.0056 | 1.0959 | 0.9125 |
| Large- | 0.0057 | 0.0028 | 2.0184 | 0.4955 |
| intestine | | | | |
| Small- | 0.0000 | 0.0107 | 0.0000 | undef |
| intestine | | | | |
| Ovary | 0.0059 | 0.0024 | 2.4887 | 0.4018 |
| Endocrine- | 0.0016 | 0.0142 | 0.1132 | 8.8363 |
| tissue | | | | |
| Brain | 0.0023 | 0.0140 | 0.1658 | 6.0318 |
| Skin | 0.0037 | 0.0000 | undef | 0.0000 |
| Hepatic | 0.0000 | 0.0063 | 0.0000 | undef |
| Heart | 0.0061 | 0.0137 | 0.4430 | 2.2572 |
| Testicles | 0.0000 | 0.0000 | undef | undef |
| Lungs | 0.0029 | 0.0074 | 0.3947 | 2.5338 |
| Stomach- | 0.0145 | 0.0000 | undef | 0.0000 |
| esophagus | | | | |
| Muscle- | 0.0017 | 0.0185 | 0.0928 | 10.7785 |
| skeleton | | | | |
| Kidneys | 0.0067 | 0.0048 | 1.3927 | 0.7180 |
| Pancreas | 0.0083 | 0.0055 | 1.4958 | 0.6685 |
| Prostate | 0.0066 | 0.0143 | 0.4604 | 2.1719 |
| T lymphoma | 0.0000 | 0.0000 | undef | undef |
| Uterus | 0.0103 | 0.0138 | 0.7497 | 1.3339 |
| White blood | 0.0007 | 0.0000 | undef | 0.0000 |
| cells | | | | |
| Hematopoetic | 0.0040 | | | |
| Penis | 0.0000 | | | |
| Seminal- | 0.0211 | | | |
| vesicles | | | | |
| Sensory organs | 0.0000 | | | |

| | FETUS |
|---|---|
| | % freq. |
| Development | 0.0000 |
| Gastrointestinal | 0.0056 |
| Brain | 0.0063 |
| Hematopoetic | 0.0000 |
| Skin | 0.0000 |
| Hepatic | 0.0000 |
| Heart blood vessels | 0.0071 |
| Lungs | 0.0000 |
| Adrenal gland | 0.0000 |
| Kidneys | 0.0000 |
| Placenta | 0.0303 |
| Prostate | 0.0000 |
| Sensory organs | 0.0000 |
| | STANDARDIZED/ |
| | SUBTRACTED LIBRARIES |
| | % freq. |
| Breast | 0.0068 |
| Breast_t | 0.0000 |
| Large intestine_t | 0.0000 |
| Ovary_n | 0.0000 |
| Ovary_t | 0.0354 |
| Endocrine tissue | 0.0245 |
| Fetal | 0.0029 |
| Gastrointestinal | 0.0122 |
| Hematopoetic | 0.0000 |
| Skin muscle | 0.0000 |
| Testicles_n | 0.0125 |
| Testicles_t | 0.0000 |
| Lungs_n | 0.0000 |
| Lungs_t | 0.0000 |
| Nerves | 0.0131 |
| Kidneys_t | 0.0000 |
| Ovary_Uterus | 0.0225 |
| Prostate_n | 0.0182 |
| Sensory organs | 0.0000 |
| White blood cells | 0.0000 |

Electronic Northern for SEQ. ID: 145

| | NORMAL | TUMOR | RATIOS | |
|---|---|---|---|---|
| | % freq. | % freq. | N/T | T/N |
| B_Lymphom | 0.0025 | 0.0000 | undef | 0.0000 |
| Bladder | 0.0390 | 0.1151 | 0.3387 | 2.9527 |
| Breast | 0.0132 | 0.0014 | 9.3932 | 0.1065 |
| Large | 0.0498 | 0.0114 | 4.3731 | 0.2287 |
| intestine | | | | |
| Small | 0.0165 | 0.0000 | undef | 0.0000 |
| intestine | | | | |
| Ovary | 0.0000 | 0.0072 | 0.0000 | undef |
| Endocrine | 0.0016 | 0.0124 | 0.1293 | 7.7318 |
| tissue | | | | |
| Brain | 0.0278 | 0.0150 | 1.8568 | 0.5386 |
| Skin | 0.0000 | 0.0000 | undef | undef |
| Hepatic | 0.0000 | 0.0127 | 0.0000 | undef |
| Heart | 0.0000 | 0.0550 | 0.0000 | undef |
| Testicles | 0.0000 | 0.0000 | undef | undef |
| Lungs | 0.0156 | 0.0000 | undef | 0.0000 |
| Stomach- | 0.0145 | 0.0064 | 2.2671 | 0.4411 |
| esophagus | | | | |
| Muscle- | 0.0017 | 0.0074 | 0.2319 | 4.3114 |
| skeleton | | | | |
| Kidneys | 0.0067 | 0.0048 | 1.3927 | 0.7180 |
| Pancreas | 0.0248 | 0.0552 | 0.4487 | 2.2285 |
| Prostate | 0.0349 | 0.0638 | 0.5463 | 1.8304 |
| T lymphoma | 0.0076 | 0.0000 | undef | 0.0000 |
| Uterus | 0.0089 | 0.0000 | undef | 0.0000 |
| White blood | 0.0157 | 0.0000 | undef | 0.0000 |
| cells | | | | |
| Hematopoetic | 0.0080 | | | |
| Penis | 0.0027 | | | |
| Seminal- | 0.0000 | | | |
| vesicles | | | | |
| Sensory organs | 0.0000 | | | |

| | FETUS |
|---|---|
| | % freq. |
| Development | 0.0000 |
| Gastrointestinal | 0.0056 |
| Brain | 0.0000 |
| Hematopoetic | 0.0079 |
| Skin | 0.0000 |
| Hepatic | 0.0000 |
| Heart blood vessels | 0.0000 |
| Lungs | 0.0000 |
| Adrenal gland | 0.0000 |
| Kidneys | 0.0124 |
| Placenta | 0.0182 |
| Prostate | 0.0000 |
| Sensory organs | 0.0000 |
| | STANDARDIZED/ |
| | SUBTRACTED LIBRARIES |
| | % freq. |
| Breast | 0.0000 |
| Breast_t | 0.0000 |
| Large intestine_t | 0.0000 |
| Ovary_n | 0.0000 |
| Ovary_t | 0.0203 |
| Endocrine tissue | 0.0000 |
| Fetal | 0.0017 |
| Gastrointestinal | 0.0000 |
| Hematopoetic | 0.0257 |
| Skin muscle | 0.0000 |
| Testicles_n | 0.0000 |
| Testicles_t | 0.0000 |
| Lungs_n | 0.0000 |
| Lungs_t | 0.0000 |
| Nerves | 0.0010 |
| Kidneys_t | 0.0000 |
| Ovary_Uterus | 0.0090 |
| Prostate_n | 0.0000 |
| Sensory organs | 0.0000 |
| White blood cells | 0.0000 |

Electronic Northern for SEQ. ID: 146

| | NORMAL | TUMOR | RATIOS | |
|---|---|---|---|---|
| | % freq. | % freq. | N/T | T/N |
| B_Lymphom | 0.0075 | 0.0000 | undef | 0.0000 |
| Bladder | 0.0078 | 0.0094 | 0.8297 | 1.2052 |
| Breast | 0.0044 | 0.0042 | 1.0437 | 0.9581 |
| Large- | 0.0038 | 0.0000 | undef | 0.0000 |
| intestine | | | | |
| Small- | 0.0165 | 0.0213 | 0.7730 | 1.2937 |
| intestine | | | | |
| Ovary | 0.0030 | 0.0048 | 0.6222 | 1.6073 |
| Endocrine- | 0.0000 | 0.0018 | 0.0000 | undef |
| tissue | | | | |
| Brain | 0.0081 | 0.0070 | 1.1605 | 0.8617 |
| Skin | 0.0037 | 0.0000 | undef | 0.0000 |
| Hepatic | 0.0093 | 0.0063 | 1.4649 | 0.6826 |
| Heart | 0.0030 | 0.0275 | 0.1108 | 9.0287 |
| Testicles | 0.0040 | 0.0059 | 0.6786 | 1.4737 |
| Lungs | 0.0000 | 0.0037 | 0.0000 | undef |
| Stomach- | 0.0000 | 0.0000 | undef | undef |
| esophagus | | | | |
| Muscle- | 0.0017 | 0.0000 | undef | 0.0000 |
| skeleton | | | | |
| Kidneys | 0.0090 | 0.0000 | undef | 0.0000 |
| Pancreas | 0.0050 | 0.0055 | 0.8975 | 1.1142 |
| Prostate | 0.0047 | 0.0091 | 0.5168 | 1.9350 |
| T lymphoma | 0.0051 | 0.0075 | 0.6762 | 1.4788 |
| Uterus | 0.0015 | 0.0046 | 0.3213 | 3.1125 |
| White blood | 0.0027 | 0.0000 | undef | 0.0000 |
| cells | | | | |
| Hematopoetic | 0.0053 | | | |
| Penis | 0.0054 | | | |
| Seminal- | 0.0070 | | | |
| vesicles | | | | |
| Sensory organs | 0.0118 | | | |

| | FETUS |
|---|---|
| | % freq. |
| Development | 0.0139 |
| Gastrointestinal | 0.0028 |
| Brain | 0.0000 |
| Hematopoetic | 0.0000 |
| Skin | 0.0000 |
| Hepatic | 0.0000 |
| Heart blood vessels | 0.0000 |
| Lungs | 0.0000 |
| Adrenal gland | 0.0000 |
| Kidneys | 0.0000 |
| Placenta | 0.0061 |
| Prostate | 0.0000 |
| Sensory organs | 0.0000 |
| | STANDARDIZED/ |
| | SUBTRACTED LIBRARIES |
| | % freq. |
| Breast | 0.0000 |
| Breast_t | 0.0000 |
| Large intestine_t | 0.0000 |
| Ovary_n | 0.0000 |
| Ovary_t | 0.0253 |
| Endocrine tissue | 0.0000 |
| Fetal | 0.0064 |
| Gastrointestinal | 0.0000 |
| Hematopoetic | 0.0257 |
| Skin muscle | 0.0000 |
| Testicles_n | 0.0084 |
| Testicles_t | 0.0000 |
| Lungs_n | 0.0195 |
| Lungs_t | 0.0000 |
| Nerves | 0.0070 |
| Kidneys_t | 0.0000 |
| Ovary_Uterus | 0.0158 |
| Prostate_n | 0.0000 |
| Sensory organs | 0.0155 |
| White blood cells | 0.0000 |

Electronic Northern for SEQ. ID: 147

| | NORMAL | TUMOR | RATIOS | |
|---|---|---|---|---|
| | % freq. | % freq. | N/T | T/N |
| B_Lymphom | 0.0025 | 0.0000 | undef | 0.0000 |
| Bladder | 0.0039 | 0.0141 | 0.2766 | 3.6156 |
| Breast | 0.0062 | 0.0028 | 2.1917 | 0.4563 |
| Large- | 0.0057 | 0.0028 | 2.0184 | 0.4955 |
| intestine | | | | |
| Small- | 0.0000 | 0.0107 | 0.0000 | undef |
| intestine | | | | |
| Ovary | 0.0059 | 0.0024 | 2.4887 | 0.4018 |
| Endocrine- | 0.0016 | 0.0106 | 0.1509 | 6.6272 |
| tissue | | | | |
| Brain | 0.0046 | 0.0080 | 0.5803 | 1.7234 |
| Skin | 0.0037 | 0.0000 | undef | 0.0000 |
| Hepatic | 0.0000 | 0.0000 | undef | undef |
| Heart | 0.0041 | 0.0137 | 0.2954 | 3.3858 |
| Testicles | 0.0000 | 0.0000 | undef | undef |
| Lungs | 0.0039 | 0.0055 | 0.7016 | 1.4253 |
| Stomach- | 0.0145 | 0.0000 | undef | 0.0000 |
| esophagus | | | | |
| Muscle- | 0.0017 | 0.0185 | 0.0928 | 10.7785 |
| skeleton | | | | |
| Kidneys | 0.0045 | 0.0048 | 0.9285 | 1.0770 |
| Pancreas | 0.0066 | 0.0055 | 1.1966 | 0.8357 |
| Prostate | 0.0057 | 0.0130 | 0.4341 | 2.3036 |
| T lymphoma | 0.0000 | 0.0000 | undef | undef |
| Uterus | 0.0074 | 0.0046 | 1.6064 | 0.6225 |
| White blood | 0.0014 | 0.0000 | undef | 0.0000 |
| cells | | | | |
| Hematopoetic | 0.0040 | | | |
| Penis | 0.0000 | | | |
| Seminal- | 0.0211 | | | |
| vesicles | | | | |
| Sensory organs | 0.0000 | | | |

| | FETUS |
|---|---|
| | % freq. |
| Development | 0.0000 |
| Gastrointestinal | 0.0056 |
| Brain | 0.0000 |
| Hematopoetic | 0.0000 |
| Skin | 0.0000 |
| Hepatic | 0.0000 |
| Heart blood vessels | 0.0071 |
| Lungs | 0.0000 |
| Adrenal gland | 0.0000 |
| Kidneys | 0.0000 |
| Placenta | 0.0121 |
| Prostate | 0.0000 |
| Sensory organs | 0.0000 |
| | STANDARDIZED/ |
| | SUBTRACTED LIBRARIES |
| | % freq. |
| Breast | 0.0068 |
| Breast_t | 0.0000 |
| Large intestine_t | 0.0000 |
| Ovary_n | 0.0000 |
| Ovary_t | 0.0304 |
| Endocrine tissue | 0.0245 |
| Fetal | 0.0046 |
| Gastrointestinal | 0.0122 |
| Hematopoetic | 0.0000 |
| Skin muscle | 0.0000 |
| Testicles_n | 0.0209 |
| Testicles_t | 0.0000 |
| Lungs_n | 0.0000 |
| Lungs_t | 0.0000 |
| Nerves | 0.0141 |
| Kidneys_t | 0.0000 |
| Ovary_Uterus | 0.0203 |
| Prostate_n | 0.0182 |
| Sensory organs | 0.0000 |
| White blood cells | 0.0000 |

Electronic Northern for SEQ. ID: 148

| | NORMAL | TUMOR | RATIOS | |
|---|---|---|---|---|
| | % freq. | % freq. | N/T | T/N |
| B_Lymphom | 0.0125 | 0.0000 | undef | 0.0000 |
| Bladder | 0.0039 | 0.0141 | 0.2766 | 3.6156 |
| Breast | 0.0079 | 0.0141 | 0.5636 | 1.7743 |
| Large- | 0.0038 | 0.0057 | 0.6728 | 1.4864 |
| intestine | | | | |
| Small- | 0.0027 | 0.0107 | 0.2577 | 3.8812 |
| intestine | | | | |
| Ovary | 0.0000 | 0.0095 | 0.0000 | undef |
| Endocrine- | 0.0161 | 0.0231 | 0.6964 | 1.4359 |
| tissue | | | | |
| Brain | 0.0156 | 0.0229 | 0.6812 | 1.4681 |
| Skin | 0.0110 | 0.0000 | undef | 0.0000 |
| Hepatic | 0.0000 | 0.0190 | 0.0000 | undef |
| Heart | 0.0132 | 0.0000 | undef | 0.0000 |
| Testicles | 0.0080 | 0.0118 | 0.6786 | 1.4737 |
| Lungs | 0.0097 | 0.0055 | 1.7540 | 0.5701 |
| Stomach- | 0.0000 | 0.0064 | 0.0000 | undef |
| esophagus | | | | |
| Muscle- | 0.0086 | 0.0000 | undef | 0.0000 |
| skeleton | | | | |
| Kidneys | 0.0246 | 0.0048 | 5.1067 | 0.1958 |
| Pancreas | 0.0116 | 0.0055 | 2.0941 | 0.4775 |
| Prostate | 0.0104 | 0.0221 | 0.4682 | 2.1360 |
| T lymphoma | 0.0152 | 0.0000 | undef | 0.0000 |
| Uterus | 0.0089 | 0.0092 | 0.9638 | 1.0375 |
| White blood | 0.0027 | 0.0000 | undef | 0.0000 |
| cells | | | | |
| Hematopoetic | 0.0107 | | | |
| Penis | 0.0054 | | | |
| Seminal- | 0.0493 | | | |
| vesicles | | | | |
| Sensory organs | 0.0000 | | | |

| | FETUS |
|---|---|
| | % freq. |
| Development | 0.0000 |
| Gastrointestinal | 0.0000 |
| Brain | 0.0125 |
| Hematopoetic | 0.0079 |
| Skin | 0.0000 |
| Hepatic | 0.0000 |
| Heart blood vessels | 0.0036 |
| Lungs | 0.0108 |
| Adrenal gland | 0.0000 |
| Kidneys | 0.0309 |
| Placenta | 0.0000 |
| Prostate | 0.0000 |
| Sensory organs | 0.0000 |
| | STANDARDIZED/ |
| | SUBTRACTED LIBRARIES |
| | % freq. |
| Breast | 0.0068 |
| Breast_t | 0.0000 |
| Large intestine_t | 0.0000 |
| Ovary_n | 0.0000 |
| Ovary_t | 0.0557 |
| Endocrine tissue | 0.0735 |
| Fetal | 0.0023 |
| Gastrointestinal | 0.0000 |
| Hematopoetic | 0.0000 |
| Skin muscle | 0.0130 |
| Testicles_n | 0.0125 |
| Testicles_t | 0.0000 |
| Lungs_n | 0.0000 |
| Lungs_t | 0.0000 |
| Nerves | 0.0020 |
| Kidneys_t | 0.0000 |
| Ovary_Uterus | 0.0248 |
| Prostate_n | 0.0121 |
| Sensory organs | 0.0000 |
| White blood cells | 0.0000 |

Electronic Northern for SEQ. ID: 149

| | NORMAL | TUMOR | RATIOS | |
|---|---|---|---|---|
| | % freq. | % freq. | N/T | T/N |
| B_Lymphom | 0.0000 | 0.0000 | undef | undef |
| Bladder | 0.0000 | 0.0000 | undef | undef |
| Breast | 0.0000 | 0.0042 | 0.0000 | undef |
| Large- | 0.0000 | 0.0028 | 0.0000 | undef |
| intestine | | | | |
| Small- | 0.0000 | 0.0000 | undef | undef |
| intestine | | | | |
| Ovary | 0.0000 | 0.0000 | undef | undef |
| Endocrine- | 0.0032 | 0.0000 | undef | 0.0000 |
| tissue | | | | |
| Brain | 0.0000 | 0.0010 | 0.0000 | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lungs | 0.0010 | 0.0000 | undef | 0.0000 |
| Stomach- | 0.0000 | 0.0000 | undef | undef |
| esophagus | | | | |
| Muscle- | 0.0000 | 0.0000 | undef | undef |
| skeleton | | | | |
| Kidneys | 0.0045 | 0.0000 | undef | 0.0000 |
| Pancreas | 0.0000 | 0.0000 | undef | undef |
| Prostate | 0.0019 | 0.0039 | 0.4823 | 2.0732 |
| T lymphoma | 0.0051 | 0.0000 | undef | 0.0000 |
| Uterus | 0.0000 | 0.0000 | undef | undef |
| White blood | 0.0014 | 0.0000 | undef | 0.0000 |
| cells | | | | |
| Hematopoetic | 0.0000 | | | |
| Penis | 0.0000 | | | |
| Seminal- | 0.0000 | | | |
| vesicles | | | | |
| Sensory organs | 0.0000 | | | |

| | FETUS |
|---|---|
| | % freq. |
| Development | 0.0000 |
| Gastrointestinal | 0.0000 |
| Brain | 0.0000 |
| Hematopoetic | 0.0079 |
| Skin | 0.0000 |
| Hepatic | 0.0000 |
| Heart blood vessels | 0.0000 |
| Lungs | 0.0000 |
| Adrenal gland | 0.0000 |
| Kidneys | 0.0000 |
| Placenta | 0.0000 |
| Prostate | 0.0000 |
| Sensory organs | 0.0000 |
| | STANDARDIZED/ |
| | SUBTRACTED LIBRARIES |
| | % freq. |
| Breast | 0.0000 |
| Breast_t | 0.0000 |
| Large intestine_t | 0.0000 |
| Ovary_n | 0.0000 |
| Ovary_t | 0.0000 |
| Endocrine tissue | 0.0000 |
| Fetal | 0.0012 |
| Gastrointestinal | 0.0244 |
| Hematopoetic | 0.0000 |
| Skin muscle | 0.0032 |
| Testicles_n | 0.0000 |
| Testicles_t | 0.0000 |
| Lungs_n | 0.0000 |
| Lungs_t | 0.0000 |
| Nerves | 0.0050 |
| Kidneys_t | 0.0000 |
| Ovary_Uterus | 0.0045 |
| Prostate_n | 0.0000 |
| Sensory organs | 0.0077 |
| White blood cells | 0.0000 |

Electronic Northern for SEQ. ID: 150

| | NORMAL | TUMOR | RATIOS | |
|---|---|---|---|---|
| | % freq. | % freq. | N/T | T/N |
| B_Lymphom | 0.0100 | 0.0000 | undef | 0.0000 |
| Bladder | 0.0117 | 0.0094 | 1.2446 | 0.8035 |
| Breast | 0.0035 | 0.0014 | 2.5048 | 0.3992 |
| Large- | 0.0038 | 0.0028 | 1.3456 | 0.7432 |
| intestine | | | | |
| Small- | 0.0055 | 0.0000 | undef | 0.0000 |
| intestine | | | | |
| Ovary | 0.0059 | 0.0048 | 1.2443 | 0.8036 |
| Endocrine- | 0.0032 | 0.0053 | 0.6036 | 1.6568 |
| tissue | | | | |
| Brain | 0.0029 | 0.0070 | 0.4145 | 2.4127 |
| Skin | 0.0000 | 0.0000 | undef | undef |
| Hepatic | 0.0000 | 0.0190 | 0.0000 | undef |
| Heart | 0.0051 | 0.0000 | undef | 0.0000 |
| Testicles | 0.0040 | 0.0000 | undef | 0.0000 |
| Lungs | 0.0039 | 0.0055 | 0.7016 | 1.4253 |
| Stomach- | 0.0000 | 0.0000 | undef | undef |
| esophagus | | | | |
| Muscle- | 0.0017 | 0.0000 | undef | 0.0000 |
| skeleton | | | | |
| Kidneys | 0.0067 | 0.0000 | undef | 0.0000 |
| Pancreas | 0.0017 | 0.0000 | undef | 0.0000 |
| Prostate | 0.0038 | 0.0052 | 0.7235 | 1.3821 |
| T lymphoma | 0.0101 | 0.0149 | 0.6762 | 1.4788 |
| Uterus | 0.0015 | 0.0046 | 0.3213 | 3.1125 |
| White blood | 0.0048 | 0.0000 | undef | 0.0000 |
| cells | | | | |
| Hematopoetic | 0.0094 | | | |
| Penis | 0.0000 | | | |
| Seminal- | 0.0000 | | | |
| vesicles | | | | |
| Sensory organs | 0.0000 | | | |

| | FETUS |
|---|---|
| | % freq. |
| Development | 0.0000 |
| Gastrointestinal | 0.0028 |
| Brain | 0.0125 |
| Hematopoetic | 0.0079 |
| Skin | 0.0000 |
| Hepatic | 0.0000 |
| Heart blood | 0.0000 |
| vessels | |
| Lungs | 0.0072 |
| Adrenal gland | 0.0000 |
| Kidneys | 0.0185 |
| Placenta | 0.0061 |
| Prostate | 0.0000 |
| Sensory organs | 0.0000 |
| | STANDARDIZED/ |
| | SUBTRACTED LIBRARIES |
| | % freq. |
| Breast | 0.0204 |
| Breast_t | 0.0000 |
| Large intestine_t | 0.0000 |
| Ovary_n | 0.0000 |
| Ovary_t | 0.0152 |
| Endocrine tissue | 0.0000 |
| Fetal | 0.0012 |
| Gastrointestinal | 0.0000 |
| Hematopoetic | 0.0257 |
| Skin muscle | 0.0000 |
| Testicles_n | 0.0042 |
| Testicles_t | 0.0000 |
| Lungs_n | 0.0000 |
| Lungs_t | 0.0000 |
| Nerves | 0.0100 |
| Kidneys_t | 0.0000 |
| Ovary_Uterus | 0.0090 |
| Prostate_n | 0.0000 |
| Sensory organs | 0.0000 |
| White blood cells | 0.0000 |

Electronic Northern for SEQ. ID: 151

| | NORMAL | TUMOR | RATIOS | |
|---|---|---|---|---|
| | % freq. | % freq. | N/T | T/N |
| B_Lymphom | 0.0000 | 0.0000 | undef | undef |
| Bladder | 0.0000 | 0.0023 | 0.0000 | undef |
| Breast | 0.0000 | 0.0000 | undef | undef |
| Large- | 0.0000 | 0.0000 | undef | undef |
| intestine | | | | |
| Small- | 0.0000 | 0.0000 | undef | undef |
| intestine | | | | |
| Ovary | 0.0000 | 0.0000 | undef | undef |
| Endocrine- | 0.0000 | 0.0071 | 0.0000 | undef |
| tissue | | | | |
| Brain | 0.0000 | 0.0010 | 0.0000 | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lungs | 0.0000 | 0.0000 | undef | undef |
| Stomach- | 0.0000 | 0.0000 | undef | undef |
| esophagus | | | | |
| Muscle- | 0.0000 | 0.0000 | undef | undef |
| skeleton | | | | |
| Kidneys | 0.0000 | 0.0000 | undef | undef |
| Pancreas | 0.0000 | 0.0000 | undef | undef |
| Prostate | 0.0009 | 0.0039 | 0.2412 | 4.1464 |
| T lymphoma | 0.0000 | 0.0075 | 0.0000 | undef |
| Uterus | 0.0015 | 0.0000 | undef | 0.0000 |
| White blood | 0.0007 | 0.0000 | undef | 0.0000 |
| cells | | | | |
| Hematopoetic | 0.0000 | | | |
| Penis | 0.0000 | | | |
| Seminal-vesicles | 0.0000 | | | |
| Sensory organs | 0.0000 | | | |

| | FETUS |
|---|---|
| | % freq. |
| Development | 0.0000 |
| Gastrointestinal | 0.0000 |
| Brain | 0.0000 |
| Hematopoetic | 0.0000 |
| Skin | 0.0000 |
| Hepatic | 0.0000 |
| Heart blood vessels | 0.0000 |
| Lungs | 0.0000 |
| Adrenal gland | 0.0000 |
| Kidneys | 0.0000 |
| Placenta | 0.0000 |
| Prostate | 0.0000 |
| Sensory organs | 0.0000 |
| | STANDARDIZED/ |
| | SUBTRACTED LIBRARIES |
| | % freq. |
| Breast | 0.0000 |
| Breast_t | 0.0000 |
| Large intestine_t | 0.0000 |
| Ovary_n | 0.0000 |
| Ovary_t | 0.0000 |
| Endocrine tissue | 0.0000 |
| Fetal | 0.0035 |
| Gastrointestinal | 0.0000 |
| Hematopoetic | 0.0000 |
| Skin muscle | 0.0032 |
| Testicles_n | 0.0000 |
| Testicles_t | 0.0000 |
| Lungs_n | 0.0000 |
| Lungs_t | 0.0000 |
| Nerves | 0.0010 |
| Kidneys_t | 0.0000 |
| Ovary_Uterus | 0.0000 |
| Prostate_n | 0.0000 |
| Sensory organs | 0.0000 |
| White blood cells | 0.0000 |

Electronic Northern for SEQ. ID: 152

| | NORMAL | TUMOR | RATIOS | |
|---|---|---|---|---|
| | % freq. | % freq. | N/T | T/N |
| B_Lymphom | 0.0000 | 0.0000 | undef | undef |
| Bladder | 0.0000 | 0.0047 | 0.0000 | undef |
| Breast | 0.0009 | 0.0014 | 0.6262 | 1.5969 |
| Large- | 0.0038 | 0.0000 | undef | 0.0000 |
| intestine | | | | |
| Small- | 0.0000 | 0.0000 | undef | undef |
| intestine | | | | |
| Ovary | 0.0000 | 0.0024 | 0.0000 | undef |
| Endocrine- | 0.0000 | 0.0000 | undef | undef |
| tissue | | | | |
| Brain | 0.0006 | 0.0010 | 0.5803 | 1.7234 |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lungs | 0.0049 | 0.0000 | undef | 0.0000 |
| Stomach- | 0.0000 | 0.0000 | undef | undef |
| esophagus | | | | |
| Muscle- | 0.0000 | 0.0000 | undef | undef |
| skeleton | | | | |
| Kidneys | 0.0000 | 0.0000 | undef | undef |
| Pancreas | 0.0017 | 0.0000 | undef | 0.0000 |
| Prostate | 0.0075 | 0.0039 | 1.9294 | 0.5183 |
| T lymphoma | 0.0000 | 0.0075 | 0.0000 | undef |
| Uterus | 0.0015 | 0.0000 | undef | 0.0000 |
| White blood | 0.0000 | 0.0000 | undef | undef |
| cells | | | | |
| Hematopoetic | 0.0000 | | | |
| Penis | 0.0027 | | | |
| Seminal- | 0.0070 | | | |
| vesicles | | | | |
| Sensory organs | 0.0000 | | | |

| | FETUS |
|---|---|
| | % freq. |
| Development | 0.0000 |
| Gastrointestinal | 0.0028 |
| Brain | 0.0063 |
| Hematopoetic | 0.0000 |
| Skin | 0.0000 |
| Hepatic | 0.0000 |
| Heart blood vessels | 0.0000 |
| Lungs | 0.0000 |
| Adrenal gland | 0.0000 |
| Kidneys | 0.0062 |
| Placenta | 0.0000 |
| Prostate | 0.0000 |
| Sensory organs | 0.0000 |
| | STANDARDIZED/ |
| | SUBTRACTED LIBRARIES |
| | % freq. |
| Breast | 0.0000 |
| Breast_t | 0.0000 |
| Large intestine_t | 0.0000 |
| Ovary_n | 0.0000 |
| Ovary_t | 0.0000 |
| Endocrine tissue | 0.0000 |
| Fetal | 0.0000 |
| Gastrointestinal | 0.0000 |
| Hematopoetic | 0.0000 |
| Skin muscle | 0.0000 |
| Testicles_n | 0.0000 |
| Testicles_t | 0.0000 |
| Lungs_n | 0.0000 |
| Lungs_t | 0.0000 |
| Nerves | 0.0010 |
| Kidneys_t | 0.0000 |
| Ovary_Uterus | 0.0000 |
| Prostate_n | 0.0000 |
| Sensory organs | 0.0000 |
| White blood cells | 0.0000 |

Electronic Northern for SEQ. ID: 153

| | NORMAL | TUMOR | RATIOS | |
|---|---|---|---|---|
| | % freq. | % freq. | N/T | T/N |
| B_Lymphom | 0.0075 | 0.0000 | undef | 0.0000 |
| Bladder | 0.0078 | 0.0000 | undef | 0.0000 |
| Breast | 0.0026 | 0.0028 | 0.9393 | 1.0646 |
| Large- | 0.0000 | 0.0000 | undef | undef |
| intestine | | | | |
| Small- | 0.0000 | 0.0000 | undef | undef |
| intestine | | | | |
| Ovary | 0.0000 | 0.0000 | undef | undef |
| Endocrine- | 0.0016 | 0.0000 | undef | 0.0000 |
| tissue | | | | |
| Brain | 0.0041 | 0.0110 | 0.3693 | 2.7082 |
| Skin | 0.0000 | 0.0000 | undef | undef |
| Hepatic | 0.0046 | 0.0000 | undef | 0.0000 |
| Heart | 0.0020 | 0.0137 | 0.1477 | 6.7715 |
| Testicles | 0.0000 | 0.0000 | undef | undef |
| Lungs | 0.0010 | 0.0018 | 0.5262 | 1.9004 |
| Stomach | 0.0000 | 0.0000 | undef | undef |
| esophagus | | | | |
| Muscle- | 0.0000 | 0.0074 | 0.0000 | undef |
| skeleton | | | | |
| Kidneys | 0.0000 | 0.0000 | undef | undef |
| Pancreas | 0.0000 | 0.0166 | 0.0000 | undef |
| Prostate | 0.0028 | 0.0078 | 0.3618 | 2.7643 |
| T lymphoma | 0.0025 | 0.0000 | undef | 0.0000 |
| Uterus | 0.0015 | 0.0046 | 0.3213 | 3.1125 |
| White blood | 0.0000 | 0.0000 | undef | undef |
| cells | | | | |
| Hematopoetic | 0.0000 | | | |
| Penis | 0.0000 | | | |
| Seminal- | 0.0000 | | | |
| vesicles | | | | |
| Sensory organs | 0.0000 | | | |

| | FETUS |
|---|---|
| | % freq. |
| Development | 0.0139 |
| Gastrointestinal | 0.0000 |
| Brain | 0.0063 |
| Hematopoetic | 0.0157 |
| Skin | 0.0000 |
| Hepatic | 0.0000 |
| Heart blood vessels | 0.0036 |
| Lungs | 0.0000 |
| Adrenal gland | 0.0000 |
| Kidneys | 0.0000 |
| Placenta | 0.0000 |
| Prostate | 0.0249 |
| Sensory organs | 0.0000 |
| | STANDARDIZED/ |
| | SUBTRACTED LIBRARIES |
| | % freq. |
| Breast | 0.0068 |
| Breast_t | 0.0000 |
| Large intestine_t | 0.0000 |
| Ovary_n | 0.0000 |
| Ovary_t | 0.0152 |
| Endocrine tissue | 0.0000 |
| Fetal | 0.0000 |
| Gastrointestinal | 0.0000 |
| Hematopoetic | 0.0000 |
| Skin muscle | 0.0000 |
| Testicles_n | 0.0000 |
| Testicles_t | 0.0000 |
| Lungs_n | 0.0000 |
| Lungs_t | 0.0000 |
| Nerves | 0.0060 |
| Kidneys_t | 0.0000 |
| Ovary Uterus | 0.0113 |
| Prostate_n | 0.0000 |
| Sensory organs | 0.0000 |
| White blood cells | 0.0000 |

Electronic Northern for SEQ. ID: 154

| | NORMAL | TUMOR | RATIOS | |
|---|---|---|---|---|
| | % freq. | % freq. | N/T | T/N |
| B_Lymphom | 0.0075 | 0.0000 | undef | 0.0000 |
| Bladder | 0.0000 | 0.0023 | 0.0000 | undef |
| Breast | 0.0018 | 0.0014 | 1.2524 | 0.7985 |
| Large- | 0.0019 | 0.0000 | undef | 0.0000 |
| intestine | | | | |
| Small- | 0.0000 | 0.0000 | undef | undef |
| intestine | | | | |
| Ovary | 0.0030 | 0.0072 | 0.4148 | 2.4110 |
| Endocrine- | 0.0000 | 0.0095 | 0.0000 | undef |
| tissue | | | | |
| Brain | 0.0018 | 0.0000 | undef | 0.0000 |
| Skin | 0.0000 | 0.0000 | undef | undef |
| Hepatic | 0.0000 | 0.0000 | undef | undef |
| Heart | 0.0010 | 0.0000 | undef | 0.0000 |
| Testicles | 0.0000 | 0.0118 | 0.0000 | undef |
| Lungs | 0.0000 | 0.0000 | undef | undef |
| Stomach- | 0.0000 | 0.0000 | undef | undef |
| esophagus | | | | |
| Muscle- | 0.0034 | 0.0000 | undef | 0.0000 |
| skeleton | | | | |
| Kidneys | 0.0000 | 0.0048 | 0.0000 | undef |
| Pancreas | 0.0000 | 0.0000 | undef | undef |
| Prostate | 0.0009 | 0.0039 | 0.2412 | 4.1465 |
| T lymphoma | 0.0025 | 0.0000 | undef | 0.0000 |
| Uterus | 0.0015 | 0.0000 | undef | 0.0000 |
| White blood | 0.0014 | 0.0000 | undef | 0.0000 |
| cells | | | | |
| Hematopoetic | 0.0053 | | | |
| Penis | 0.0027 | | | |
| Seminal- | 0.0000 | | | |
| vesicles | | | | |
| Sensory organs | 0.0000 | | | |

| | FETUS |
|---|---|
| | % freq. |
| Development | 0.0000 |
| Gastrointestinal | 0.0000 |
| Brain | 0.0000 |
| Hematopoetic | 0.0000 |
| Skin | 0.0000 |
| Hepatic | 0.0000 |
| Heart blood vessels | 0.0000 |
| Lungs | 0.0000 |
| Adrenal gland | 0.0000 |
| Kidneys | 0.0000 |
| Placenta | 0.0000 |
| Prostate | 0.0000 |
| Sensory organs | 0.0000 |
| | STANDARDIZED/ |
| | SUBTRACTED LIBRARIES |
| | % freq. |
| Breast | 0.0000 |
| Breast_t | 0.0000 |
| Large intestine_t | 0.0000 |
| Ovary_n | 0.0000 |
| Ovary_t | 0.0000 |
| Endocrine tissue | 0.0000 |
| Fetal | 0.0017 |
| Gastrointestinal | 0.0000 |
| Hematopoetic | 0.0000 |
| Skin muscle | 0.0000 |
| Testicles_n | 0.0042 |
| Testicles_t | 0.0000 |
| Lungs_n | 0.0000 |
| Lungs_t | 0.0000 |
| Nerves | 0.0060 |
| Kidneys_t | 0.0000 |
| Ovary_Uterus | 0.0000 |
| Prostate_n | 0.0000 |
| Sensory organs | 0.0000 |
| White blood cells | 0.0000 |

Electronic Northern for SEQ. ID: 155

| | NORMAL | TUMOR | RATIOS | |
|---|---|---|---|---|
| | % freq. | % freq. | N/T | T/N |
| B_Lymphom | 0.0100 | 0.0000 | undef | 0.0000 |
| Bladder | 0.0000 | 0.0117 | 0.0000 | undef |
| Breast | 0.0088 | 0.0098 | 0.8946 | 1.1179 |
| Large- | 0.0019 | 0.0028 | 0.6728 | 1.4864 |
| intestine | | | | |
| Small- | 0.0027 | 0.0000 | undef | 0.0000 |
| intestine | | | | |
| Ovary | 0.0119 | 0.0048 | 2.4886 | 0.4018 |
| Endocrine- | 0.0064 | 0.0019 | 3.3917 | 0.2948 |
| tissue | | | | |
| Brain | 0.0205 | 0.0060 | 3.4255 | 0.2919 |
| Skin | 0.0000 | 0.0000 | undef | undef |
| Hepatic | 0.0046 | 0.0063 | 0.7324 | 1.3653 |
| Heart | 0.0030 | 0.0000 | undef | 0.0000 |
| Testicles | 0.0040 | 0.0000 | undef | 0.0000 |
| Lungs | 0.0049 | 0.0037 | 1.3155 | 0.7602 |
| Stomach- | 0.0072 | 0.0192 | 0.3778 | 2.6472 |
| esophagus | | | | |
| Muscle- | 0.0103 | 0.0037 | 2.7833 | 0.3593 |
| skeleton | | | | |
| Kidneys | 0.0022 | 0.0096 | 0.2321 | 4.3085 |
| Pancreas | 0.0000 | 0.0000 | undef | undef |
| Prostate | 0.0066 | 0.0130 | 0.5065 | 1.9745 |
| T lymphoma | 0.0000 | 0.0075 | 0.0000 | undef |
| Uterus | 0.0046 | 0.0138 | 0.3368 | 2.9694 |
| White blood | 0.0027 | 0.0000 | undef | 0.0000 |
| cells | | | | |
| Hematopoetic | 0.0040 | | | |
| Penis | 0.0080 | | | |
| Seminal- | 0.0070 | | | |
| vesicles | | | | |
| Sensory organs | 0.0235 | | | |

| | FETUS |
|---|---|
| | % freq. |
| Development | 0.0139 |
| Gastrointestinal | 0.0083 |
| Brain | 0.0626 |
| Hematopoetic | 0.0118 |
| Skin | 0.0000 |
| Hepatic | 0.0000 |
| Heart blood vessels | 0.0107 |
| Lungs | 0.0108 |
| Adrenal gland | 0.0254 |
| Kidneys | 0.0124 |
| Placenta | 0.0061 |
| Prostate | 0.0000 |
| Sensory organs | 0.0000 |
| | STANDARDIZED/ |
| | SUBTRACTED LIBRARIES |
| | % freq. |
| Breast | 0.0544 |
| Breast_t | 0.0000 |
| Large intestine_t | 0.0000 |
| Ovary_n | 0.0000 |
| Ovary_t | 0.0051 |
| Endocrine tissue | 0.0979 |
| Fetal | 0.0058 |
| Gastrointestinal | 0.0122 |
| Hematopoetic | 0.0000 |
| Skin muscle | 0.0194 |
| Testicles_n | 0.0167 |
| Testicles_t | 0.0000 |
| Lungs_n | 0.0000 |
| Lungs_t | 0.0000 |
| Nerves | 0.0201 |
| Kidneys_t | 0.0000 |
| Ovary_Uterus | 0.0090 |
| Prostate_n | 0.0000 |
| Sensory organs | 0.0000 |
| White blood cells | 0.0000 |

Electronic Northern for SEQ. ID: 156

| | NORMAL | TUMOR | RATIOS | |
|---|---|---|---|---|
| | % freq. | % freq. | N/T | T/N |
| B_Lymphom | 0.0025 | 0.0000 | undef | 0.0000 |
| Bladder | 0.0039 | 0.0141 | 0.2766 | 3.6156 |
| Breast | 0.0044 | 0.0098 | 0.4473 | 2.2357 |
| Large- | 0.0019 | 0.0057 | 0.3364 | 2.9727 |
| intestine | | | | |
| Small- | 0.0110 | 0.0107 | 1.0306 | 0.9703 |
| intestine | | | | |
| Ovary | 0.0030 | 0.0119 | 0.2489 | 4.0182 |
| Endocrine- | 0.0032 | 0.0089 | 0.3621 | 2.7613 |
| tissue | | | | |
| Brain | 0.0017 | 0.0040 | 0.4352 | 2.2978 |
| Skin | 0.0037 | 0.0789 | 0.0466 | 21.4787 |
| Hepatic | 0.0000 | 0.0063 | 0.0000 | undef |
| Heart | 0.0152 | 0.0000 | undef | 0.0000 |
| Testicles | 0.0120 | 0.0237 | 0.5089 | 1.9650 |
| Lungs | 0.0039 | 0.0037 | 1.0524 | 0.9502 |
| Stomach- | 0.0000 | 0.0000 | undef | undef |
| esophagus | | | | |
| Muscle- | 0.0086 | 0.0000 | undef | 0.0000 |
| skeleton | | | | |
| Kidneys | 0.0090 | 0.0000 | undef | 0.0000 |
| Pancreas | 0.0017 | 0.0055 | 0.2992 | 3.3427 |
| Prostate | 0.0038 | 0.0117 | 0.3216 | 3.1098 |
| T lymphoma | 0.0101 | 0.0224 | 0.4508 | 2.2182 |
| Uterus | 0.0030 | 0.0092 | 0.3213 | 3.1125 |
| White blood | 0.0123 | 0.0607 | 0.2029 | 4.9287 |
| cells | | | | |
| Hematopoetic | 0.0040 | | | |
| Penis | 0.0054 | | | |
| Seminal- | 0.0000 | | | |
| vesicles | | | | |
| Sensory organs | 0.0000 | | | |

| | FETUS |
|---|---|
| | % freq. |
| Development | 0.0000 |
| Gastrointestinal | 0.0028 |
| Brain | 0.0000 |
| Hematopoetic | 0.0079 |
| Skin | 0.0000 |
| Hepatic | 0.0000 |
| Heart blood vessels | 0.0142 |
| Lungs | 0.0072 |
| Adrenal gland | 0.0000 |
| Kidneys | 0.0000 |
| Placenta | 0.0061 |
| Prostate | 0.0000 |
| Sensory organs | 0.0126 |
| | STANDARDIZED/ |
| | SUBTRACTED LIBRARIES |
| | % freq. |
| Breast | 0.0000 |
| Breast_t | 0.0000 |
| Large intestine_t | 0.0000 |
| Ovary_n | 0.0000 |
| Ovary_t | 0.0000 |
| Endocrine tissue | 0.0000 |
| Fetal | 0.0070 |
| Gastrointestinal | 0.0122 |
| Hematopoetic | 0.0000 |
| Skin muscle | 0.0065 |
| Testicles_n | 0.0042 |
| Testicles_t | 0.0000 |
| Lungs_n | 0.0098 |
| Lungs_t | 0.0000 |
| Nerves | 0.0060 |
| Kidneys_t | 0.0000 |
| Ovary Uterus | 0.0023 |
| Prostate_n | 0.0121 |
| Sensory organs | 0.0000 |
| White blood cells | 0.0000 |

Electronic Northern for SEQ. ID: 157

| | NORMAL | TUMOR | RATIOS | |
|---|---|---|---|---|
| | % freq. | % freq. | N/T | T/N |
| B_Lymphom | 0.0025 | 0.0000 | undef | 0.0000 |
| Bladder | 0.0000 | 0.0235 | 0.0000 | undef |
| Breast | 0.0035 | 0.0070 | 0.5010 | 1.9961 |
| Large- | 0.0134 | 0.0000 | undef | 0.0000 |
| intestine | | | | |
| Small- | 0.0000 | 0.0000 | undef | undef |
| intestine | | | | |
| Ovary | 0.0030 | 0.0048 | 0.6222 | 1.6073 |
| Endocrine- | 0.0032 | 0.0000 | undef | 0.0000 |
| tissue | | | | |
| Brain | 0.0006 | 0.0040 | 0.1451 | 6.8935 |
| Skin | 0.0037 | 0.0000 | undef | 0.0000 |
| Hepatic | 0.0000 | 0.0000 | undef | undef |
| Heart | 0.0010 | 0.0275 | 0.0369 | 27.0862 |
| Testicles | 0.0000 | 0.0000 | undef | undef |
| Lungs | 0.0010 | 0.0000 | undef | 0.0000 |
| Stomach- | 0.0072 | 0.0064 | 1.1335 | 0.8822 |
| esophagus | | | | |
| Muscle- | 0.0017 | 0.0000 | undef | 0.0000 |
| skeleton | | | | |
| Kidneys | 0.0022 | 0.0048 | 0.4642 | 2.1540 |
| Pancreas | 0.0000 | 0.0055 | 0.0000 | undef |
| Prostate | 0.0123 | 0.0456 | 0.2687 | 3.7211 |
| T lymphoma | 0.0000 | 0.0000 | undef | undef |
| Uterus | 0.0015 | 0.0046 | 0.3213 | 3.1125 |
| White blood | 0.0000 | 0.0000 | undef | undef |
| cells | | | | |
| Hematopoetic | 0.0013 | | | |
| Penis | 0.0000 | | | |
| Seminal- | 0.0070 | | | |
| vesicles | | | | |
| Sensory organs | 0.0000 | | | |

| | FETUS |
|---|---|
| | % freq. |
| Development | 0.0000 |
| Gastrointestinal | 0.0028 |
| Brain | 0.0000 |
| Hematopoetic | 0.0000 |
| Skin | 0.0000 |
| Hepatic | 0.0000 |
| Heart blood vessels | 0.0000 |
| Lungs | 0.0072 |
| Adrenal gland | 0.0000 |
| Kidneys | 0.0124 |
| Placenta | 0.0000 |
| Prostate | 0.0000 |
| Sensory organs | 0.0000 |
| | STANDARDIZED/ |
| | SUBTRACTED LIBRARIES |
| | % freq. |
| Breast | 0.0000 |
| Breast_t | 0.0000 |
| Large intestine_t | 0.0000 |
| Ovary_n | 0.0000 |
| Ovary_t | 0.0051 |
| Endocrine tissue | 0.0000 |
| Fetal | 0.0000 |
| Gastrointestinal | 0.0000 |
| Hematopoetic | 0.0000 |
| Skin muscle | 0.0032 |
| Testicles_n | 0.0042 |
| Testicles_t | 0.0000 |
| Lungs_n | 0.0098 |
| Lungs_t | 0.0000 |
| Nerves | 0.0040 |
| Kidneys_t | 0.0000 |
| Ovary_Uterus | 0.0045 |
| Prostate_n | 0.0000 |
| Sensory organs | 0.0000 |
| White blood cells | 0.0000 |

Electronic Northern for SEQ. ID: 158

| | NORMAL | TUMOR | RATIOS | |
|---|---|---|---|---|
| | % freq. | % freq. | N/T | T/N |
| B_Lymphom | 0.0000 | 0.0000 | undef | undef |
| Bladder | 0.0039 | 0.0047 | 0.8297 | 1.2052 |
| Breast | 0.0079 | 0.0112 | 0.7045 | 1.4195 |
| Large- | 0.0287 | 0.0142 | 2.0184 | 0.4955 |
| intestine | | | | |
| Small- | 0.0192 | 0.0000 | undef | 0.0000 |
| intestine | | | | |
| Ovary | 0.0059 | 0.0167 | 0.3555 | 2.8128 |
| Endocrine- | 0.0064 | 0.0177 | 0.3621 | 2.7613 |
| tissue | | | | |
| Brain | 0.0087 | 0.0140 | 0.6217 | 1.6085 |
| Skin | 0.0000 | 0.0000 | undef | undef |
| Hepatic | 0.0139 | 0.0063 | 2.1973 | 0.4551 |
| Heart | 0.0020 | 0.0412 | 0.0492 | 20.3146 |
| Testicles | 0.0040 | 0.0000 | undef | 0.0000 |
| Lungs | 0.0039 | 0.0129 | 0.3007 | 3.3256 |
| Stomach- | 0.0217 | 0.0000 | undef | 0.0000 |
| esophagus | | | | |
| Muscle- | 0.0120 | 0.0037 | 3.2472 | 0.3080 |
| skeleton | | | | |
| Kidneys | 0.0045 | 0.0048 | 0.9285 | 1.0770 |
| Pancreas | 0.0050 | 0.0221 | 0.2244 | 4.4570 |
| Prostate | 0.0254 | 0.0469 | 0.5426 | 1.8428 |
| T lymphoma | 0.0051 | 0.0075 | 0.6762 | 1.4788 |
| Uterus | 0.0030 | 0.0092 | 0.3213 | 3.1125 |
| White blood | 0.0027 | 0.0000 | undef | 0.0000 |
| cells | | | | |
| Hematopoetic | 0.0040 | | | |
| Penis | 0.0054 | | | |
| Seminal- | 0.0000 | | | |
| vesicles | | | | |
| Sensory organs | 0.0000 | | | |

| | FETUS |
|---|---|
| | % freq. |
| Development | 0.0278 |
| Gastrointestinal | 0.0056 |
| Brain | 0.0125 |
| Hematopoetic | 0.0039 |
| Skin | 0.0000 |
| Hepatic | 0.0000 |
| Heart blood vessels | 0.0036 |
| Lungs | 0.0000 |
| Adrenal gland | 0.0507 |
| Kidneys | 0.0000 |
| Placenta | 0.0000 |
| Prostate | 0.0000 |
| Sensory organs | 0.0000 |
| | STANDARDIZED/ |
| | SUBTRACTED LIBRARIES |
| | % freq. |
| Breast | 0.0136 |
| Breast_t | 0.0000 |
| Large intestine_t | 0.0000 |
| Ovary_n | 0.0000 |
| Ovary_t | 0.0253 |
| Endocrine tissue | 0.0000 |
| Fetal | 0.0075 |
| Gastrointestinal | 0.0244 |
| Hematopoetic | 0.0000 |
| Skin muscle | 0.0097 |
| Testicles_n | 0.0000 |
| Testicles_t | 0.0000 |
| Lungs_n | 0.0098 |
| Lungs_t | 0.0000 |
| Nerves | 0.0141 |
| Kidneys_t | 0.0000 |
| Ovary_Uterus | 0.0158 |
| Prostate_n | 0.0243 |
| Sensory organs | 0.0000 |
| White blood cells | 0.0000 |

Electronic Northern for SEQ. ID: 159

| | NORMAL | TUMOR | RATIOS | |
|---|---|---|---|---|
| | % freq. | % freq. | N/T | T/N |
| B_Lymphom | 0.0075 | 0.0000 | undef | 0.0000 |
| Bladder | 0.0000 | 0.0141 | 0.0000 | undef |
| Breast | 0.0026 | 0.0028 | 0.9393 | 1.0646 |
| Large- | 0.0077 | 0.0057 | 1.3456 | 0.7432 |
| intestine | | | | |
| Small- | 0.0027 | 0.0107 | 0.2577 | 3.8812 |
| intestine | | | | |
| Ovary | 0.0030 | 0.0095 | 0.3111 | 3.2146 |
| Endocrine- | 0.0064 | 0.0018 | 3.6214 | 0.2761 |
| tissue | | | | |
| Brain | 0.0000 | 0.0060 | 0.0000 | undef |
| Skin | 0.0073 | 0.0000 | undef | 0.0000 |
| Hepatic | 0.0000 | 0.0190 | 0.0000 | undef |
| Heart | 0.0020 | 0.0000 | undef | 0.0000 |
| Testicles | 0.0000 | 0.0059 | 0.0000 | undef |
| Lungs | 0.0010 | 0.0037 | 0.2631 | 3.8007 |
| Stomach- | 0.0072 | 0.0064 | 1.1335 | 0.8822 |
| esophagus | | | | |
| Muscle- | 0.0000 | 0.0000 | undef | undef |
| skeleton | | | | |
| Kidneys | 0.0067 | 0.0048 | 1.3927 | 0.7180 |
| Pancreas | 0.0050 | 0.0000 | undef | 0.0000 |
| Prostate | 0.0123 | 0.0260 | 0.4703 | 2.1264 |
| T lymphoma | 0.0101 | 0.0149 | 0.6762 | 1.4788 |
| Uterus | 0.0044 | 0.0092 | 0.4819 | 2.0750 |
| White blood | 0.0027 | 0.0000 | undef | 0.0000 |
| cells | | | | |
| Hematopoetic | 0.0120 | | | |
| Penis | 0.0000 | | | |
| Seminal- | 0.0000 | | | |
| vesicles | | | | |
| Sensory organs | 0.0000 | | | |

| | FETUS |
|---|---|
| | % freq. |
| Development | 0.0139 |
| Gastrointestinal | 0.0194 |
| Brain | 0.0063 |
| Hematopoetic | 0.0000 |
| Skin | 0.0000 |
| Hepatic | 0.0260 |
| Heart blood vessels | 0.0071 |
| Lungs | 0.0108 |
| Adrenal gland | 0.0000 |
| Kidneys | 0.0000 |
| Placenta | 0.0121 |
| Prostate | 0.0499 |
| Sensory organs | 0.0000 |
| | STANDARDIZED/ |
| | SUBTRACTED LIBRARIES |
| | % freq. |
| Breast | 0.0000 |
| Breast_t | 0.0000 |
| Large intestine_t | 0.0000 |
| Ovary_n | 0.0000 |
| Ovary_t | 0.0608 |
| Endocrine tissue | 0.0000 |
| Fetal | 0.0075 |
| Gastrointestinal | 0.0000 |
| Hematopoetic | 0.0000 |
| Skin muscle | 0.0000 |
| Testicles_n | 0.0000 |
| Testicles_t | 0.0000 |
| Lungs_n | 0.0000 |
| Lungs_t | 0.0000 |
| Nerves | 0.0030 |
| Kidneys_t | 0.0000 |
| Ovary_Uterus | 0.0293 |
| Prostate_n | 0.0061 |
| Sensory organs | 0.0387 |
| White blood cells | 0.0000 |

Electronic Northern for SEQ. ID: 160

| | NORMAL | TUMOR | RATIOS | |
|---|---|---|---|---|
| | % freq. | % freq. | N/T | T/N |
| B_Lymphom | 0.0125 | 0.0000 | undef | 0.0000 |
| Bladder | 0.0000 | 0.0023 | 0.0000 | undef |
| Breast | 0.0044 | 0.0056 | 0.7828 | 1.2775 |
| Large- | 0.0019 | 0.0028 | 0.6728 | 1.4864 |
| intestine | | | | |
| Small- | 0.0055 | 0.0000 | undef | 0.0000 |
| intestine | | | | |
| Ovary | 0.0059 | 0.0048 | 1.2443 | 0.8036 |
| Endocrine- | 0.0048 | 0.0071 | 0.6790 | 1.4727 |
| tissue | | | | |
| Brain | 0.0017 | 0.0050 | 0.3482 | 2.8723 |
| Skin | 0.0037 | 0.0000 | undef | 0.0000 |
| Hepatic | 0.0093 | 0.0000 | undef | 0.0000 |
| Heart | 0.0071 | 0.0275 | 0.2584 | 3.8695 |
| Testicles | 0.0040 | 0.0059 | 0.6786 | 1.4737 |
| Lungs | 0.0097 | 0.0074 | 1.3155 | 0.7601 |
| Stomach- | 0.0072 | 0.0064 | 1.1335 | 0.8822 |
| esophagus | | | | |
| Muscle- | 0.0069 | 0.0037 | 1.8555 | 0.5389 |
| skeleton | | | | |
| Kidneys | 0.0045 | 0.0048 | 0.9285 | 1.0770 |
| Pancreas | 0.0000 | 0.0000 | undef | undef |
| Prostate | 0.0009 | 0.0104 | 0.0904 | 11.0571 |
| T lymphoma | 0.0025 | 0.0000 | undef | 0.0000 |
| Uterus | 0.0059 | 0.0092 | 0.6426 | 1.5563 |
| White blood | 0.0000 | 0.0000 | undef | undef |
| cells | | | | |
| Hematopoetic | 0.0040 | | | |
| Penis | 0.0000 | | | |
| Seminal- | 0.0000 | | | |
| vesicles | | | | |
| Sensory organs | 0.0000 | | | |

| | FETUS |
|---|---|
| | % freq. |
| Development | 0.0000 |
| Gastrointestinal | 0.0083 |
| Brain | 0.0000 |
| Hematopoetic | 0.0118 |
| Skin | 0.0000 |
| Hepatic | 0.0000 |
| Heart blood vessels | 0.0178 |
| Lungs | 0.0181 |
| Adrenal gland | 0.0000 |
| Kidneys | 0.0247 |
| Placenta | 0.0061 |
| Prostate | 0.0249 |
| Sensory organs | 0.0000 |
| | STANDARDIZED/ |
| | SUBTRACTED LIBRARIES |
| | % freq. |
| Breast | 0.0136 |
| Breast_t | 0.0000 |
| Large intestine_t | 0.0000 |
| Ovary_n | 0.0000 |
| Ovary_t | 0.0000 |
| Endocrine tissue | 0.0000 |
| Fetal | 0.0110 |
| Gastrointestinal | 0.0000 |
| Hematopoetic | 0.0000 |
| Skin muscle | 0.0194 |
| Testicles_n | 0.0000 |
| Testicles_t | 0.0000 |
| Lungs_n | 0.0293 |
| Lungs_t | 0.0000 |
| Nerves | 0.0020 |
| Kidneys_t | 0.0000 |
| Ovary_Uterus | 0.0023 |
| Prostate_n | 0.0000 |
| Sensory organs | 0.0000 |
| White blood cells | 0.0000 |

Electronic Northern for SEQ. ID: 161

| | NORMAL | TUMOR | RATIOS | |
|---|---|---|---|---|
| | % freq. | % freq. | N/T | T/N |
| B_Lymphom | 0.0000 | 0.0000 | undef | undef |
| Bladder | 0.0000 | 0.0070 | 0.0000 | undef |
| Breast | 0.0000 | 0.0014 | 0.0000 | undef |
| Large- | 0.0000 | 0.0000 | undef | undef |
| intestine | | | | |
| Small- | 0.0000 | 0.0000 | undef | undef |
| intestine | | | | |
| Ovary | 0.0000 | 0.0000 | undef | undef |
| Endocrine- | 0.0000 | 0.0000 | undef | undef |
| tissue | | | | |
| Brain | 0.0006 | 0.0030 | 0.1934 | 5.1701 |
| Skin | 0.0037 | 0.0000 | undef | 0.0000 |
| Hepatic | 0.0000 | 0.0063 | 0.0000 | undef |
| Heart | 0.0000 | 0.0000 | undef | undef |
| Testicles | 0.0040 | 0.0000 | undef | 0.0000 |
| Lungs | 0.0000 | 0.0000 | undef | undef |
| Stomach- | 0.0072 | 0.0000 | undef | 0.0000 |
| esophagus | | | | |
| Muscle- | 0.0000 | 0.0000 | undef | undef |
| skeleton | | | | |
| Kidneys | 0.0000 | 0.0000 | undef | undef |
| Pancreas | 0.0000 | 0.0000 | undef | undef |
| Prostate | 0.0038 | 0.0117 | 0.3216 | 3.1098 |
| T lymphoma | 0.0025 | 0.0000 | undef | 0.0000 |
| Uterus | 0.0015 | 0.0092 | 0.1606 | 6.2251 |
| White blood | 0.0000 | 0.0000 | undef | undef |
| cells | | | | |
| Hematopoetic | 0.0000 | | | |
| Penis | 0.0000 | | | |
| Seminal- | 0.0000 | | | |
| vesicles | | | | |
| Sensory organs | 0.0000 | | | |

| | FETUS |
|---|---|
| | % freq. |
| Development | 0.0000 |
| Gastrointestinal | 0.0028 |
| Brain | 0.0000 |
| Hematopoetic | 0.0039 |
| Skin | 0.0000 |
| Hepatic | 0.0000 |
| Heart blood vessels | 0.0000 |
| Lungs | 0.0000 |
| Adrenal gland | 0.0000 |
| Kidneys | 0.0000 |
| Placenta | 0.0000 |
| Prostate | 0.0000 |
| Sensory organs | 0.0000 |
| | STANDARDIZED/ |
| | SUBTRACTED LIBRARIES |
| | % freq. |
| Breast | 0.0000 |
| Breast_t | 0.0000 |
| Large intestine_t | 0.0000 |
| Ovary_n | 0.0000 |
| Ovary_t | 0.0000 |
| Endocrine tissue | 0.0000 |
| Fetal | 0.0012 |
| Gastrointestinal | 0.0000 |
| Hematopoetic | 0.0000 |
| Skin muscle | 0.0000 |
| Testicles_n | 0.0000 |
| Testicles_t | 0.0000 |
| Lungs_n | 0.0098 |
| Lungs_t | 0.0000 |
| Nerves | 0.0000 |
| Kidneys_t | 0.0000 |
| Ovary_Uterus | 0.0000 |
| Prostate_n | 0.0000 |
| Sensory organs | 0.0000 |
| White blood cells | 0.0000 |

Electronic Northern for SEQ. ID: 162

| | NORMAL | TUMOR | RATIOS | |
|---|---|---|---|---|
| | % freq. | % freq. | N/T | T/N |
| B_Lymphom | 0.0075 | 0.0000 | undef | 0.0000 |
| Bladder | 0.0039 | 0.0070 | 0.5532 | 1.8078 |
| Breast | 0.0053 | 0.0028 | 1.8786 | 0.5323 |
| Large- | 0.0038 | 0.0057 | 0.6728 | 1.4864 |
| intestine | | | | |
| Small- | 0.0082 | 0.0000 | undef | 0.0000 |
| intestine | | | | |
| Ovary | 0.0030 | 0.0048 | 0.6222 | 1.6073 |
| Endocrine- | 0.0016 | 0.0018 | 0.9054 | 1.1045 |
| tissue | | | | |
| Brain | 0.0214 | 0.0030 | 7.1565 | 0.1397 |
| Skin | 0.0000 | 0.0000 | undef | undef |
| Hepatic | 0.0000 | 0.0063 | 0.0000 | undef |
| Heart | 0.0030 | 0.0000 | undef | 0.0000 |
| Testicles | 0.0040 | 0.0000 | undef | 0.0000 |
| Lungs | 0.0019 | 0.0092 | 0.2105 | 4.7509 |
| Stomach- | 0.0000 | 0.0000 | undef | undef |
| esophagus | | | | |
| Muscle- | 0.0069 | 0.0074 | 0.9278 | 1.0778 |
| skeleton | | | | |
| Kidneys | 0.0022 | 0.0000 | undef | 0.0000 |
| Pancreas | 0.0017 | 0.0055 | 0.2992 | 3.3427 |
| Prostate | 0.0066 | 0.0143 | 0.4604 | 2.1719 |
| T lymphoma | 0.0000 | 0.0000 | undef | undef |
| Uterus | 0.0015 | 0.0046 | 0.3213 | 3.1125 |
| White blood | 0.0068 | 0.0000 | undef | 0.0000 |
| cells | | | | |
| Hematopoetic | 0.0040 | | | |
| Penis | 0.0054 | | | |
| Seminal- | 0.0141 | | | |
| vesicles | | | | |
| Sensory organs | 0.0000 | | | |

| | FETUS |
|---|---|
| | % freq. |
| Development | 0.0000 |
| Gastrointestinal | 0.0056 |
| Brain | 0.0000 |
| Hematopoetic | 0.0039 |
| Skin | 0.0000 |
| Hepatic | 0.0000 |
| Heart blood vessels | 0.0036 |
| Lungs | 0.0036 |
| Adrenal gland | 0.0000 |
| Kidneys | 0.0000 |
| Placenta | 0.0000 |
| Prostate | 0.0000 |
| Sensory organs | 0.0000 |
| | STANDARDIZED/ |
| | SUBTRACTED LIBRARIES |
| | % freq. |
| Breast | 0.0340 |
| Breast_t | 0.0000 |
| Large intestine_t | 0.0000 |
| Ovary_n | 0.0000 |
| Ovary_t | 0.0051 |
| Endocrine tissue | 0.0000 |
| Fetal | 0.0035 |
| Gastrointestinal | 0.0366 |
| Hematopoetic | 0.0000 |
| Skin muscle | 0.0097 |
| Testicles_n | 0.0000 |
| Testicles_t | 0.0000 |
| Lungs_n | 0.0098 |
| Lungs_t | 0.0000 |
| Nerves | 0.0201 |
| Kidneys_t | 0.0000 |
| Ovary_Uterus | 0.0045 |
| Prostate_n | 0.0000 |
| Sensory organs | 0.0000 |
| White blood cells | 0.0000 |

Electronic Northern for SEQ. ID: 163

| | NORMAL | TUMOR | RATIOS | |
|---|---|---|---|---|
| | % freq. | % freq. | N/T | T/N |
| B_Lymphom | 0.0050 | 0.0000 | undef | 0.0000 |
| Bladder | 0.0078 | 0.0117 | 0.6638 | 1.5065 |
| Breast | 0.0070 | 0.0028 | 2.5048 | 0.3992 |
| Large- | 0.0038 | 0.0028 | 1.3456 | 0.7432 |
| intestine | | | | |
| Small- | 0.0055 | 0.0000 | undef | 0.0000 |
| intestine | | | | |
| Ovary | 0.0030 | 0.0024 | 1.2443 | 0.8036 |
| Endocrine- | 0.0096 | 0.0106 | 0.9054 | 1.1045 |
| tissue | | | | |
| Brain | 0.0046 | 0.0130 | 0.3571 | 2.8005 |
| Skin | 0.0000 | 0.0000 | undef | undef |
| Hepatic | 0.0046 | 0.0000 | undef | 0.0000 |
| Heart | 0.0061 | 0.0000 | undef | 0.0000 |
| Testicles | 0.0040 | 0.0059 | 0.6786 | 1.4737 |
| Lungs | 0.0068 | 0.0018 | 3.6835 | 0.2715 |
| Stomach- | 0.0145 | 0.0064 | 2.2671 | 0.4411 |
| esophagus | | | | |
| Muscle- | 0.0000 | 0.0000 | undef | undef |
| skeleton | | | | |
| Kidneys | 0.0022 | 0.0096 | 0.2321 | 4.3081 |
| Pancreas | 0.0050 | 0.0110 | 0.4487 | 2.2285 |
| Prostate | 0.0028 | 0.0065 | 0.4341 | 2.3036 |
| T lymphoma | 0.0025 | 0.0149 | 0.1691 | 5.9152 |
| Uterus | 0.0030 | 0.0000 | undef | 0.0000 |
| White blood | 0.0027 | 0.0000 | undef | 0.0000 |
| cells | | | | |
| Hematopoetic | 0.0040 | | | |
| Penis | 0.0027 | | | |
| Seminal- | 0.0070 | | | |
| vesicles | | | | |
| Sensory organs | 0.0000 | | | |

| | FETUS |
|---|---|
| | % freq. |
| Development | 0.0000 |
| Gastrointestinal | 0.0111 |
| Brain | 0.0063 |
| Hematopoetic | 0.0000 |
| Skin | 0.0000 |
| Hepatic | 0.0000 |
| Heart blood vessels | 0.0107 |
| Lungs | 0.0036 |
| Adrenal gland | 0.0000 |
| Kidneys | 0.0000 |
| Placenta | 0.0121 |
| Prostate | 0.0000 |
| Sensory organs | 0.0000 |
| | STANDARDIZED/ |
| | SUBTRACTED LIBRARIES |
| | % freq. |
| Breast | 0.0136 |
| Breast_t | 0.0000 |
| Large intestine_t | 0.0000 |
| Ovary_n | 0.0000 |
| Ovary_t | 0.0203 |
| Endocrine tissue | 0.0000 |
| Fetal | 0.0139 |
| Gastrointestinal | 0.0122 |
| Hematopoetic | 0.0257 |
| Skin muscle | 0.0130 |
| Testicles_n | 0.0125 |
| Testicles_t | 0.0000 |
| Lungs_n | 0.0098 |
| Lungs_t | 0.0000 |
| Nerves | 0.0100 |
| Kidneys_t | 0.0000 |
| Ovary_Uterus | 0.0180 |
| Prostate_n | 0.0000 |
| Sensory organs | 0.0000 |
| White blood cells | 0.0000 |

Electronic Northern for SEQ. ID: 164

| | NORMAL | TUMOR | RATIOS | |
|---|---|---|---|---|
| | % freq. | % freq. | N/T | T/N |
| B_Lymphom | 0.0000 | 0.0000 | undef | undef |
| Bladder | 0.0078 | 0.0023 | 3.3190 | 0.3013 |
| Breast | 0.0053 | 0.0126 | 0.4175 | 2.3954 |
| Large- | 0.0057 | 0.0057 | 1.0092 | 0.9909 |
| intestine | | | | |
| Small- | 0.0110 | 0.0000 | undef | 0.0000 |
| intestine | | | | |
| Ovary | 0.0119 | 0.0119 | 0.9955 | 1.0046 |
| Endocrine- | 0.0032 | 0.0035 | 0.9054 | 1.1045 |
| tissue | | | | |
| Brain | 0.0174 | 0.0040 | 4.3519 | 0.2298 |
| Skin | 0.0000 | 0.0000 | undef | undef |
| Hepatic | 0.0093 | 0.0063 | 1.4649 | 0.6826 |
| Heart | 0.0112 | 0.0000 | undef | 0.0000 |
| Testicles | 0.0120 | 0.0059 | 2.0357 | 0.4912 |
| Lungs | 0.0068 | 0.0148 | 0.4604 | 2.1719 |
| Stomach- | 0.0000 | 0.0000 | undef | undef |
| esophagus | | | | |
| Muscle- | 0.0069 | 0.0037 | 1.8555 | 0.5389 |
| skeleton | | | | |
| Kidneys | 0.0022 | 0.0048 | 0.4642 | 2.1540 |
| Pancreas | 0.0066 | 0.0000 | undef | 0.0000 |
| Prostate | 0.0019 | 0.0052 | 0.3618 | 2.7643 |
| T lymphoma | 0.0126 | 0.0373 | 0.3381 | 2.9576 |
| Uterus | 0.0030 | 0.0092 | 0.3213 | 3.1125 |
| White blood | 0.0027 | 0.0000 | undef | 0.0000 |
| cells | | | | |
| Hematopoetic | 0.0053 | | | |
| Penis | 0.0000 | | | |
| Seminal- | 0.0000 | | | |
| vesicles | | | | |
| Sensory organs | 0.0000 | | | |

| | FETUS |
|---|---|
| | % freq. |
| Development | 0.0000 |
| Gastrointestinal | 0.0083 |
| Brain | 0.0125 |
| Hematopoetic | 0.0000 |
| Skin | 0.0000 |
| Hepatic | 0.0260 |
| Heart blood vessels | 0.0036 |
| Lungs | 0.0181 |
| Adrenal gland | 0.0000 |
| Kidneys | 0.0062 |
| Placenta | 0.0061 |
| Prostate | 0.0000 |
| Sensory organs | 0.0126 |
| | STANDARDIZED/ |
| | SUBTRACTED LIBRARIES |
| | % freq. |
| Breast | 0.0000 |
| Breast_t | 0.0000 |
| Large intestine_t | 0.0000 |
| Ovary_n | 0.1595 |
| Ovary_t | 0.0000 |
| Endocrine tissue | 0.0000 |
| Fetal | 0.0035 |
| Gastrointestinal | 0.0000 |
| Hematopoetic | 0.0000 |
| Skin muscle | 0.0065 |
| Testicles_n | 0.0042 |
| Testicles_t | 0.0000 |
| Lungs_n | 0.0293 |
| Lungs_t | 0.0000 |
| Nerves | 0.0090 |
| Kidneys_t | 0.0000 |
| Ovary_Uterus | 0.0023 |
| Prostate_n | 0.0182 |
| Sensory organs | 0.0000 |
| White blood cells | 0.0000 |

2.2. Fisher Test

In order to decide whether a partial sequence S of a gene occurs significantly more often or less often in a library for normal tissue than in a library for degenerated tissue, Fisher's exact test, a standard statistical process, is carried out (Hays, W. L., (1991) Statistics, Harcourt Brace College Publishers, Fort Worth).

The null hypothesis reads: The two libraries cannot be distinguished with respect to the frequency of sequences homologous to S. If the null hypothesis can be rejected with high enough certainty, the gene belonging to S is accepted as an advantageous candidate for a cancer gene, and in the next step an attempt is made to achieve lengthening of its sequence.

### EXAMPLE 3

### Automatic Lengthening of the Partial Sequence

Automatic lengthening of partial sequence S is completed in three steps:

1. Determination of all sequences homologous to S from the total set of available sequences using BLAST.

2. Assembling these sequences by means of the standard program GAP4 (Bonfield, J. K., Smith, K. F. and Staden, R. (1995), Nucleic Acids Research 23 4992-4999) (contig formation).

3. Computation of a consensus sequence C from the assembled sequences.

Consensus sequence C will generally be longer than initial sequence S. Its electronic Northern Blot will accordingly deviate from that for S. A repeated Fisher test decides whether the alternative hypothesis of deviation from a uniform expression in the two libraries can be maintained. If this is the case, an attempt is made to lengthen C in the same way as S. This iteration is continued with the consensus sequences Cᵢ (i: iteration index) obtained each time until the alternative hypothesis is rejected (if H₀ Exit; truncation criterion I) or until automatic lengthening is no longer possible (while Cᵢ>Cᵢ₋₁; truncation criterion II).

In the case of truncation criterion II, with the consensus sequence present after the last iteration, a complete or roughly complete sequence of a gene that can be related to cancer with high statistical certainty is acquired.

Analogously to the above described examples, the nucleic acid sequences described in Table I from prostate tumor tissue were found.

Furthermore, for the individual nucleic acid sequences the peptide sequences (ORFs) which are listed in Table II were determined, in which no peptide can be assigned to a few nucleic acid sequences and more than one peptide can be assigned to some nucleic acid sequences. As already mentioned above, both the determined nucleic acid sequences and also the peptide sequences assigned to the nucleic acid sequences are the subject of this invention.

### EXAMPLE 4

### Mapping of Nucleic Acid Sequences on the Human Genome

Human genes were mapped using the Stanford G3 Hybrid Panel (Stewart et al., 1997) which is marketed by Research Genetics, Huntsville, Ala. This panel consists of 83 different genomic DNAs of human-hamster hybrid cell lines and allows resolution of 500 kilobases. The hybrid cell lines were obtained by fusion of irradiated diploid human cells with cells of the Chinese hamster. The retention pattern of the human chromosome fragments is determined by means of gene-specific primers in a polymerase chain reaction and is analyzed using software available from the Stanford RH server (http://www.stanford.edu/RH/rhserver_form2. html). This program determines the STS marker which is nearest the desired gene. The corresponding cytogenetic band was determined using the “Mapview” program of the Genome Database (GDB), (http://gdbwww.dkfz-heidelberg.de). In addition to mapping of genes on the human chromosome set by various experimental methods it is possible to determine the location of genes on this by biocomputer methods. To do this, the known program e-PCR was used (Schuler G D (1998) Electronic PCR: Bridging the Gap Between Genome Mapping and Genome Sequencing. Trends Biotechnol 16: 456-459, Schuler G D (1997). Sequence Mapping By Electronic PCR, Genome Res. 7; 541-550). The database used here no longer corresponds to the one cited in the literature, but is a development that includes data from the public database RHdb (http://www.ebi.ac.uk/RHdb/index.html). Analogously to the mapping by the hybrid panels, the results were evaluated with the aforementioned software and the software of the Whitehead Institute (http://carbon.wi.mit.edu:8000/cgi-bin/contig/rhmapper.pl).

### EXAMPLE 5

### Obtaining Genomic DNA Sequences (BAC Clones)

The genomic BAC clones containing the corresponding cDNAs (http://www.tree.caltech.edu/; Shizuya, H., B. Birren, U-J. Kim, V. Mancino, T. Slepak, Y. Tachiiri, M. Simon (1992) Proc. Natl. Acad. Sci., USA 89: 8794-8797) were isolated with the procedure of “down-to-the-well”. In this procedure a library consisting of BAC clones (the library covers roughly 3× the human genome) is moved into a certain raster so that the DNA of these clones with a specific PCR can be studied. In doing so “pooling” of the DNA of different BAC clones takes place. Combinatorial analysis makes it possible to determine the clones that contain the desired DNA. By fixing the clones the address of the clones in the library can be determined. This address together with the name of the library which is being used unequivocally fixes the clones and thus the DNA sequence of these clones.

The following examples explain the successful isolation of the genomic BAC clones without limiting them thereto.

The libraries used were CITB B and CITB C:

| Seq.ID No. | Identified BACs | | | | |
|---|---|---|---|---|---|
| 3 | 523-P-21 | | | | |
| 20 | 446-E-12 | | | | |
| 33 | 544-K-9 | 552-M-9 | 391-B15 | 228-G-2 | |
| 36 | 420-I-3 | 395-G-4 | 489-A14 | | |
| 44 | 194-A-22 | 438-F-10 | 491-A-16 | | |
| 50 | 423-P-13 | 327-G-1 | 221-M-24 | 197-M-19 | |
| 51 | 244-C-10 | 421-G-19 | | | |
| 53 | 342-M-13 | 347-N-18 | | | |
| 142 | 353-J-9 | 383-F-17 | 404-H-16 | 416-N-2 | 501-A-5 |

TABLE 1

| | | | | | | | Length of | Seq. ID |
|---|---|---|---|---|---|---|---|---|
| | | | | | | Length of | Claimed | of |
| Seq. | | | | Cytogenetic | Nearest | Initial EST | Sequence | Initial |
| ID | Expression | Function | Modules | Localization | Marker | in Bases | in Bases | Sequence |
| 3 | elevated in | Human homolog to | Pyropho | 10q21.1- | SHGC-36251- | 246 | 1258 | |
| | prostate tumors | bovine inorganic | sphatase | q22.1 | D10S1428 | | | |
| | | pyrophosphatase | | | | | | |
| 4 | elevated in | Unknown | | 2q35 | SHGC-32022- | 257 | 894 | |
| | prostate tumors | | | | D2S126 | | | |
| 6 | elevated in | Human homolog to | TBC | | | 225 | 1017 | |
| | prostate tumors | a GTPase activating | | | | | | |
| | | protein from | | | | | | |
| | | Yarrowia lypolytica | | | | | | |
| 7 | elevated in | MLN 62 | MATH; | 17p12-q11.2 | D17S798 | 192 | 671 | |
| | prostate and | | TRAF | | | | | |
| | breast tumors | | | | | | | |
| 10 | elevated in | Unknown | | 1p36.13- | D1S3153- | 225 | 870 | |
| | prostate tumors | | | p36.12 | D1S2740 | | | |
| 12 | elevated in | BCE-1 | | 2q35 | SHGC-32022- | 215 | 1312 | |
| | prostate tumors | | | | D2S126 | | | |
| 13 | elevated in | GATA-3 | | 10p15.1 | D10S1431- | 230 | 1008 | |
| | prostate tumors | | | | SHGC-32335 | | | |
| 15 | elevated in | Unknown | | 19p13.3- | D19S854- | 193 | 706 | |
| | prostate tumors | | | P13.2 | SHGC-36605 | | | |
| 17 | in | Unknown | | 2q33.1-q33.3 | SHGC-36529- | 246 | 450 | |
| | nonstandardized | | | | D2S374 | | | |
| | or subtracted | | | | | | | |
| | libraries only | | | | | | | |
| | detectable in | | | | | | | |
| | prostate and | | | | | | | |
| | brain tumors | | | | | | | |
| 34 | elevated in | Unknown | | 17q11.2 | SHGC-2348 | 386 | 1563 | |
| | prostate tumors | | | | | | | |
| 36 | ca. 5x more | Homolog to NMDA | | 8q24.3 | AFMA128XH5 | 298 | 1557 | |
| | heavily in | | | | | | | |
| | prostate tumors | | | | | | | |
| | than in | | | | | | | |
| | corresponding | | | | | | | |
| | normal tissue | | | | | | | |
| 37 | 5x more heavily | Unknown | | 18q21.1- | D18S831- | 213 | 1381 | |
| | in prostate | | | 18q21.33 | D18S64 | | | |
| | tumors than in | | | | | | | |
| | corresponding | | | | | | | |
| | normal tissue | | | | | | | |
| 39 | ca. 4.5x more | Human homolog to | | 3q22.1-3q23 | SHGC-37132- | 224 | 615 | |
| | heavily in | Sec61 | | | SHGC-30693 | | | |
| | prostate tumors | | | | | | | |
| | than in | | | | | | | |
| | corresponding | | | | | | | |
| | normal tissue | | | | | | | |
| 40 | 8x more heavily | HUMAN INTESTINAL | trefoil | 21q22.3 | | 276 | 834 | |
| | in prostate | TREFOIL FACTOR | | | | | | |
| | tumors than in | HP1.B | | | | | | |
| | corresponding | | | | | | | |
| | normal tissue | | | | | | | |
| 44 | elevated in | Unknown | | 6q12-p12.1 | SHGC-10670- | 207 | 997 | |
| | prostate and | | | | SHGC-31710 | | | |
| | skin tumors | | | | | | | |
| 45 | elevated in | Homolog to human | CATION_{—} | 19q13.2- | SHGC-9668- | 212 | 548 | |
| | prostate tumors | metastatin 1 | CHAN | q13.41 | SHGC-30929 | | | |
| | | | NEL_TRPL | | | | | |
| 46 | elevated in | Unknown | | 9q21.32- | D9S1120- | 241 | 1448 | |
| | prostate tumors | | | q22.1 | D9S1812 | | | |
| 47 | elevated in | Homolog to a | | 16p13.3- | D16S521- | 165 | 1163 | |
| | prostate tumors | protein kinase C | | q24.3 | SHGC-2951 | | | |
| | | substrate | | | | | | |
| 48 | elevated in | Unknown | | 19q13.2- | NIB1805-WI- | 260 | 906 | |
| | prostate tumors | | | q13.41 | 9028 | | | |
| 49 | elevated in | Unknown | | 2p24.3 | D2S1288 | 259 | 1222 | |
| | prostate tumors | | | | | | | |
| 50 | elevated in | Unknown | | | | 277 | 649 | |
| | prostate tumors | | | | | | | |
| 51 | elevated in | Unknown | | | | 272 | 1226 | |
| | prostate tumors | | | | | | | |
| 52 | elevated in | Unknown | | 12q24.22- | D12S1619- | 249 | 1036 | |
| | prostate tumors | | | q24.23 | D12S1915 | | | |
| 53 | elevated in | Homology to the | Ribosomal_{—} | 10q11.1- | D10S1571- | 228 | 758 | |
| | prostate tumors | family of high | L7A | q23.1 | D10S583 | | | |
| | | mobility group | | | | | | |
| | | proteins | | | | | | |
| 142 | elevated in | Calcyclin- | | 1q24-25 | WI-3733-WI- | 317 | 1663 | |
| | prostate tumors | binding protein | | | 5780 | | | |
| 144 | expressed | Unknown | | 2q35 | SHGC-32022- | | 2105 | 4 |
| | 2.1719x in the | | | | D2S126 | | | |
| | prostate tumor | | | | | | | |
| | compared to | | | | | | | |
| | normal prostate | | | | | | | |
| | tissue | | | | | | | |
| 145 | expressed | Human homolog to | TBC | | | | 1125 | 6 |
| | 1.8304x in the | a GTPase | | | | | | |
| | prostate tumor | activating | | | | | | |
| | compared to | protein from | | | | | | |
| | normal prostate | Yarrowia | | | | | | |
| | tissue | lypolytica | | | | | | |
| 146 | expressed | Unknown | | 1p36.l3- | D1S3153- | | 1490 | 10 |
| | 1.935x in the | | | p36.12 | D1S2740 | | | |
| | prostate tumor | | | | | | | |
| | compared to | | | | | | | |
| | normal prostate | | | | | | | |
| | tissue | | | | | | | |
| 147 | expressed BCE-1 | | | 2q35 | SHGC-32022- | | 1692 | 12 |
| | 2.3036x in the | | | | D2S126 | | | |
| | prostate tumor | | | | | | | |
| | compared to | | | | | | | |
| | normal prostate | | | | | | | |
| | tissue | | | | | | | |
| 148 | expressed | Unknown | | 19p13.3- | D19S854- | | 866 | 15 |
| | 2.1361x in the | | | p13.2 | SHGC-36605 | | | |
| | prostate tumor | | | | | | | |
| | compared to | | | | | | | |
| | normal prostate | | | | | | | |
| | tissue | | | | | | | |
| 149 | expressed | Unknown | | 2q33.1-q33.3 | SHGC-36529- | | 992 | 17 |
| | 2.0732x in the | | | | D2S374 | | | |
| | prostate tumor | | | | | | | |
| | compared to | | | | | | | |
| | normal prostate | | | | | | | |
| | tissue | | | | | | | |
| 150 | expressed | Human homolog to | GAMMA_{—} | | | | 1640 | 20 |
| | 1.3821x in the | tyrosine-protein | CARB | | | | | |
| | prostate tumor | kinase 1 from | OXYLASE | | | | | |
| | compared to | Dictyostelium | | | | | | |
| | normal prostate | discoideurm | | | | | | |
| | tissue | | | | | | | |
| 151 | expressed | Human homolog to | zf-C2H2 | 16p11.2 | SHGC-2727- | | 974 | 22 |
| | 4.1464x in the | ZPF-29 from mice | | | SHGC-35326 | | | |
| | prostate tumor | | | | | | | |
| | compared to | | | | | | | |
| | normal prostate | | | | | | | |
| | tissue | | | | | | | |
| 152 | expressed | Homolog to | | 15q25.1- | SHGC-30592- | | 1059 | 27 |
| | 1.9294x in the | DYNANIN-1 | | q25.3 | SHGC-35120 | | | |
| | prostate tumor | | | | | | | |
| | compared to | | | | | | | |
| | normal prostate | | | | | | | |
| | tissue | | | | | | | |
| 153 | expressed | Unknown | | 10q21.1-21.3 | D10S1688- | | 2003 | 31 |
| | 2.7643x in the | | | | SHGC-33649 | | | |
| | prostate tumor | | | | | | | |
| | compared to | | | | | | | |
| | normal prostate | | | | | | | |
| | tissue | | | | | | | |
| 154 | expressed | Human homolog to | HSP70 | | | | 1130 | 33 |
| | 4.1465x in the | NST-1 from mice | | | | | | |
| | prostate tumor | | | | | | | |
| | compared to | | | | | | | |
| | normal prostate | | | | | | | |
| | tissue | | | | | | | |
| 155 | expressed | Phosphatidyl | | 17q11.2 | SHGC-2348 | | 5779 | 34 |
| | 1.9745x. in the | inositol-4- | | | | | | |
| | prostate tumor | phosphate-5- | | | | | | |
| | compared to | kinase type 2 | | | | | | |
| | normal prostate | beta | | | | | | |
| | tissue | | | | | | | |
| 156 | expressed | Lysophospholipase | ESTERASE | 6q12-p12.1 | SHGC-10670- | | 2408 | 44 |
| | 3.1098x in the | | | | SHGC-31710 | | | |
| | prostate tumor | | | | | | | |
| | compared to | | | | | | | |
| | normal prostate | | | | | | | |
| | tissue | | | | | | | |
| 157 | expressed | Homolog to human | CATION_{—} | 19q13.2- | SHGC-9668- | | 1548 | 45 |
| | 3.7211x in the | metastatin 1 | CHANNEL- | q13.41 | SHGC-30929 | | | |
| | prostate tumor | | TRPL | | | | | |
| | compared to | | | | | | | |
| | normal prostate | | | | | | | |
| | tissue | | | | | | | |
| 158 | expressed | Unknown | | 9q21.32- | D9S1120- | | 2319 | 46 |
| | 1.8428x in the | | | q22.1 | D9S1812 | | | |
| | prostate tumor | | | | | | | |
| | compared to | | | | | | | |
| | normal prostate | | | | | | | |
| | tissue | | | | | | | |
| 18 | elevated in | Unknown | | Xq25-28 | DXS1062- | 233 | 418 | |
| | prostate tumors | | | | DXS9754 | | | |
| 19 | only in | Homolog to | | 20q13.2 | D20S197- | 282 | 549 | |
| | prostate and | inositol | | | SHGC-33731 | | | |
| | breast tumors | polyphosphate-4- | | | | | | |
| | | phosphatase type | | | | | | |
| | | 1 beta | | | | | | |
| 20 | elevated in | Unknown | | | | 249 | 999 | |
| | prostate tumors | | | | | | | |
| 21 | elevated in | Unknown | | 4q31.22- | D4S397- | 301 | 311 | |
| | prostate tumors | | | 31.23 | D4S2965 | | | |
| 22 | elevated in | Unknown | | 16p11.2 | SHGC-2727- | 231 | 527 | |
| | prostate tumors | | | | SHGC-35326 | | | |
| 23 | detectable only | Unknown | | 9q21.31- | SHGC-35114- | 224 | 359 | |
| | in prostate | | | 21.32 | D9S1122 | | | |
| | tumors | | | | | | | |
| 24 | elevated in | Unknown | | 2p11.1-2p12 | D2S388- | 242 | 1482 | |
| | prostate tumors | | | | D2S2579 | | | |
| 26 | detectable only | Unknown | | 2q11.2 | D2S2311- | 229 | 316 | |
| | in prostate | | | | D2S2886 | | | |
| | tumors | | | | | | | |
| 27 | elevated in | Homolog to | | 15q25.1- | SHGC-30592- | 209 | 369 | |
| | prostate tumor | DYNAMIN-1 | | q25.3 | SHGC-35120 | | | |
| | and hyperplasia | | | | | | | |
| 29 | detectable in | Unknown | | 6q23.1-23.3 | D6S1835- | 260 | 591 | |
| | normal lung | | | | D6S1569 | | | |
| | tissue, in | | | | | | | |
| | prostate and | | | | | | | |
| | endocrine | | | | | | | |
| | tumors | | | | | | | |
| 31 | elevated in | Unknown | | 10q21.1-21.3 | D10S1688- | 241 | 1631 | |
| | prostate tumors | | | | SHGC-33649 | | | |
| 33 | elevated in | Human homolog to | HSP70 | | | 247 | 844 | |
| | prostate tumors | NST-1 from mice | | | | | | |
| 159 | expressed | Unknown | | 19q13.2- | NIB1805-WI- | | 1467 | 48 |
| | 2.1264x in the | | | q13.41 | 9028 | | | |
| | prostate tumor | | | | | | | |
| | compared to | | | | | | | |
| | normal prostate | | | | | | | |
| | tissue | | | | | | | |
| 160 | expressed | Unknown | | 2p24.3 | D2S1288 | | 1348 | 49 |
| | 11.0571x in the | | | | | | | |
| | prostate tumor | | | | | | | |
| | compared to | | | | | | | |
| | normal prostate | | | | | | | |
| | tissue | | | | | | | |
| 161 | expressed | Unknown | | | | | 1290 | 51 |
| | 3.1098x in the | | | | | | | |
| | prostate tumor | | | | | | | |
| | compared to | | | | | | | |
| | normal prostate | | | | | | | |
| | tissue | | | | | | | |
| 162 | expressed | Unknown | | 12q24.22- | D12S1619- | | 2912 | 52 |
| | 2.1719x in the | | | q24.23 | D12S1915 | | | |
| | prostate tumor | | | | | | | |
| | compared to | | | | | | | |
| | normal prostate | | | | | | | |
| | tissue | | | | | | | |
| 163 | expressed | Homology to the | Ribosom | 10q11.1- | D10S1571- | | 850 | 53 |
| | 2.3036x in the | family of high | al_{—} | q23.1 | D10S583 | | | |
| | prostate tumor | mobility group | L7Ae | | | | | |
| | compared to | proteins | | | | | | |
| | normal prostate | | | | | | | |
| | tissue | | | | | | | |
| 164 | expressed | Calcyclin- | | 1q24-25 | WI-3733-WI- | | 2223 | 142 |
| | 2.7643x in the | binding protein | | | 5780 | | | |
| | prostate tumor | | | | | | | |
| | compared to | | | | | | | |
| | normal prostate | | | | | | | |
| | tissue | | | | | | | |

TABLE 2

| DNA Sequence | Peptide | Sequence | | | | | |
|---|---|---|---|---|---|---|---|
| Seq ID No. | (ORFs) | Seq ID No. | | | | | |
| 3 | 57 | | | | | | |
| 4 | 58 | | | | | | |
| 6 | 59 | 60 | | | | | |
| 7 | 61 | | | | | | |
| 10 | 64 | | | | | | |
| 12 | 66 | 67 | | | | | |
| 13 | 68 | | | | | | |
| 15 | 70 | 71 | | | | | |
| 17 | 73 | 74 | | | | | |
| 18 | 75 | | | | | | |
| 19 | 76 | 77 | | | | | |
| 20 | 78 | 79 | | | | | |
| 21 | 80 | | | | | | |
| 22 | 81 | | | | | | |
| 23 | 82 | | | | | | |
| 24 | 83 | | | | | | |
| 26 | 85 | | | | | | |
| 27 | 86 | | | | | | |
| 29 | 87 | | | | | | |
| 31 | 89 | 90 | | | | | |
| 33 | | | | | | | |
| 34 | 92 | | | | | | |
| 36 | 94 | | | | | | |
| 37 | 95 | | | | | | |
| 39 | | | | | | | |
| 40 | 97 | | | | | | |
| 44 | 101 | 102 | 103 | 104 | 105 | | |
| 45 | 106 | 107 | 108 | | | | |
| 46 | 109 | 110 | 111 | | | | |
| 47 | 112 | 113 | 114 | | | | |
| 48 | 115 | 116 | 117 | 118 | 119 | 120 | 121 |
| 49 | 122 | 123 | 124 | 125 | | | |
| 50 | 126 | 127 | 128 | 129 | | | |
| 51 | 130 | 131 | 132 | | | | |
| 52 | 133 | 134 | 135 | 136 | 137 | | |
| 53 | 138 | 139 | 140 | 141 | | | |
| 142 | 143 | | | | | | |
| 144 | 165 | 166 | | | | | |
| 145 | 167 | | | | | | |
| 146 | 168 | 169 | | | | | |
| 147 | 170 | 171 | | | | | |
| 148 | 172 | 173 | | | | | |
| 149 | 174 | 175 | | | | | |
| 150 | 176 | 177 | | | | | |
| 151 | 178 | | | | | | |
| 152 | 179 | 180 | | | | | |
| 153 | 181 | 182 | | | | | |
| 154 | 183 | | | | | | |
| 155 | 184 | | | | | | |
| 156 | 185 | | | | | | |
| 157 | 186 | 187 | | | | | |
| 158 | 188 | 189 | | | | | |
| 159 | 190 | 191 | | | | | |
| 160 | 192 | 193 | | | | | |
| 161 | 194 | 195 | | | | | |
| 162 | 196 | 197 | | | | | |
| 163 | 198 | | | | | | |
| 164 | 199 | | | | | | |

The inventive nucleic acid sequences Seq. ID No. 3, 4, 6, 7, 10, 12, 13, 15, 17-24, 26, 27, 29, 31-34, 36, 37, 39, 40, 44-53; 142 144-164 of the determined candidate genes and the determined amino acid sequences Seq. ID No. 57-61, 64, 66-68, 70-71, 73-83, 85-87, 89-90, 92, 94, 95, 97, 101-141, 143 and 165-199 are described in the following sequence protocol.

1. A nucleic acid sequence that codes a gene product or a part thereof, comprising
a) a nucleic acid sequence selected from the group Seq. ID No. 3, 4, 6; 10, 12, 15, 17-24, 27, 29, 31-34, 36, 39, 44-53; 142, 144-164
b) an allelic variation of the nucleic acid sequences named under a) or
c) a nucleic acid sequence that is complementary to the nucleic acid sequences named under a) or b).

2. A nucleic acid sequence according to one of the sequences Seq. ID No. 3, 4, 6; 10, 12, 15, 17-24, 27, 29, 31-34, 36, 39, 44-53; 142, 144-164, or a complementary or allelic variant thereof.

3. Nucleic acid sequence Seq. ID No. 3, 4, 6, 7, 10, 12, 13, 15, 17-24, 26, 27, 29, 31-34, 36, 37, 39, 40, 44-53; 142, 144-164, characterized in that it is expressed elevated in prostate tumor tissue.

4. BAC, PAC and Cosmid clones containing functional genes and their chromosomal localization according to sequences Seq. ID No. 3, 4, 6; 10, 12, 15, 17-24, 27, 29, 31-34, 36, 39, 44-53; 142, 144-164 for use as a vehicle for gene transfer.

5. A nucleic acid sequence according to claims 1 to 4, wherein it has 90% homology to a human nucleic acid sequence.

6. A nucleic acid sequence according to claims 1 to 4, wherein it has 95% homology to a human nucleic acid sequence.

7. A nucleic acid sequence comprising a portion of the nucleic acid sequences named in claims 1 to 6, in such a sufficient amount that they hybridize with the sequences according to claims 1 to 6.

8. A nucleic acid sequence according to claims 1 to 7, wherein the size of the fragment has a length of at least 50 to 2000 bp.

9. A nucleic acid sequence according to claims 1 to 7, wherein the size of the fragment has a length of at least 150 to 1700 bp.

10. A nucleic acid sequence according to one of claims 1 to 9, which codes at least one partial sequence of a bioactive polypeptide.

11. An expression cassette comprising a nucleic acid fragment or a sequence according to one of claims 1 to 9, together with at least one control or regulatory sequence.

12. An expression cassette, comprising a nucleic acid fragment or a sequence according to claim 11, in which the control or regulatory sequence is a suitable promoter.

13. An expression cassette according to one of claims 11 and 12, wherein the DNA sequences located on the cassette code a fusion protein that comprises a known protein and a bioactive polypeptide fragment.

14. Use of nucleic acid sequences according to claims 1 to 10 for producing full-length genes.

15. A DNA fragment, comprising a gene, which can be obtained from an application according to claim 14.

16. Host cell, containing as the heterologous part of its expressible genetic information a nucleic acid fragment according to one of claims 1 to 10.

17. Host cell according to claim 16, wherein it is a prokaryotic or eukaryotic cell system.

18. Host cell according to one of claims 16 or 17, wherein the prokaryotic cell system is E. coli and the eukaryotic cell system is an animal, human or yeast cell system.

19. A process for producing a polypeptide or a fragment, wherein the host cells according to claims 16 to 18 are cultivated.

20. An antibody which is directed against a polypeptide or a fragment, which by the nucleic acids of sequences Seq. ID No. 3, 4, 6; 10, 12, 15, 17-24, 27, 29, 31-34, 36, 39, 44-53; 142, 144-164, which can be obtained according to claim 19.

21. An antibody according to claim 20, wherein it is monoclonal.

22. A protein according to claim 20, wherein it originates from a phage display.

23. Polypeptide partial sequences according to sequences Seq. ID No. 57-61, 64, 66-68, 70-71, 73-83, 85-87, 89-90, 92, 94, 95, 97, 101-141, 143 and 165-199.

24. Polypeptide partial sequences according to claim 22, with at least 80% homology to these sequences.

25. Polypeptide partial sequences according to claim 22, with at least 90% homology to these sequences.

26. Use of polypeptide partial sequences according to sequences Seq. ID No. 57-61, 64, 66-68, 70-71, 73-83, 85-87, 89-90, 92, 94, 95, 97, 101-141, 143 and 165-199 as tools for finding active agents against prostate cancer.

27. Use of nucleic acid sequences according to sequences Seq. ID No. 3, 4, 6; 10, 12, 15, 17-24, 27, 29, 31-34, 36, 39, 44-53; 142, 144-164 for expression of polypeptides which can be used as tools for finding active agents against prostate cancer.

28. Use of nucleic acid sequences Seq. ID No. 3, 4, 6; 10, 12, 15, 17-24, 27-29, 31-34, 36, 39, 44 53; 142, 144-164 in sense or antisense form.

29. Use of polypeptide partial sequences Seq. ID No. 57-61, 64, 66-68, 70-71, 73-83, 85-87, 89-90, 92, 94, 95, 97, 101-141, 143 and 165-199 as pharmaceutical agents in gene therapy for treatment of prostate cancer.

30. Use of polypeptide partial sequences Seq. ID No. 57-61, 64, 66-68, 70-71, 73-83, 85-87, 89-90, 92, 94, 95, 97, 101-141, 143 and 165-199 to produce a pharmaceutical agent for treatment of prostate cancer.

31. Pharmaceutical agent, containing at least one polypeptide partial sequence Seq. ID No. 57-61, 64, 66-68, 70-71, 73-83, 85-87, 89-90, 92, 94, 95, 97, 101-141, 143 and 165-199.

32. A nucleic acid sequence according to claims 1 to 10, wherein it is a genomic sequence.

33. A nucleic acid sequence according to claims 1 to 10, wherein it is an mRNA sequence.

34. Genomic genes, their promoters, enhancers, silencers, exon structure, intron structure and their splice variants, obtainable from cDNAs of sequences Seq. ID No. 3, 4, 6; 10, 12, 15, 17-24, 27, 29, 31-34, 36, 39, 44-53; 142, 144-164.

35. Use of the genomic genes according to claim 33, together with suitable regulatory elements.

36. Use according to claim 34, wherein the regulatory element is a suitable promoter and/or enhancer.


SEQUENCE LISTING (201 sequences)

SEQ ID NO: 1-2 - skipped (2 entries)

SEQ ID NO: 3 - DNA, 1200 bp, Homo sapiens
gggctctctccttgtcagtcggcgccgcgtgcgggctggtggctctgtggcagcggcggcggcaggactccggcactatgagcggcttcagcaccgaggagcgcgccgcgccttctccctggagtaccgagtcttcctcaaaaatgagaaaggacaatatatatctccatttcatgatattccaatttatgcagataaggatgtgtttcacatggtagttgaagtaccacgctggtctaatgcaaaaatggagattgctacaaaggaccctttaaaccctattaaacaagatgtgaaaaaaggaaaacttcgctatgttgcgaatttgttcccgtataaaggatatatctggaactatggtgccatccctcagacttgggaagacccagggcacaatgataaacatactggctgttgtggtgacaatgacccaattgatgtgtgtgaaattggaagcaaggtatgtgcaagaggtgaaataattggcgtgaaagttctaggcatattggctatgattgacgaaggggaaaccgactggaaagtcattgccattaatgtggatgatcctgatgcagccaattataatgatatcaatgatgtcaaacggctgaaacctggctacttagaagctactgtggactggtttagaaggtataaggttcctgatggaaaaccagaaaatgagtttgcgtttaatgcagaatttaaagataaggactttgccattgatattattaaaagcactcatgaccattggaaagcattagtgactaagaaaacgaatggaaaaggaatcagttgcatgaatacaactttgtctgagagccccttcaagtgtgatcctgatgctgccagagccattgtggatgctttaccaccaccctgtgaatctgcctgcacagtaccaacagacgtggataagtggttccatcaccagaaaaactaatgagatttctctggaatacaagctgatattgctacatcgtgttcatctggatgtattagaagtaaaagtagtagcttttcaaagctttaaatttgtagaactcatctaactaaagtaaattctgctgtgactaatccaatatactcagaatgttatccatctaaagcatttttcatatctcaactaagataacttttagcacatgcttaaatatcaaaggagttgccattttggaggcacttgtgaataggtgt

SEQ ID NO: 4 - DNA, 894 bp, Homo sapiens
cttcacggagcagcatgctttctcctgcgccgatggctcaccccgctgcccactgcgtggggctgaccgggctgatgtggccgatgttctggggacagctctagaggagctgaaccgccgctaccacccggccttgcggctccagaagcagcagctggtgaatggctaccgacgctttgatccggcccggggtatggaatacacgctggacttgcagctggaggcactgaccccccagggaggccgccggcccctcactcgccgagtgcagctgctccggccgctgagccgcgtggagatcttgcctgtgccctatgtcactgaggcctcacgtctcactgtgctgctgcctctagctgcggctgagcgtgacctggcccctggcttcttggaggcctttgccactgcagcactggagcctggtgatgctgcggcagccctgaccctgctgctactgtatgagccgcgccaggcccagcgcgtggcccatgcagatgtcttcgcacctgtcaaggcccatgtggcagagctggagcggcgtttccccggtgcccgggtgccatggctcagtgtgcagacagccgcaccctcaccactacgcctcatggatctactctccaagaagcacccgctggacacactgttcctgctggccgggccagacacggtgctcacgcctgacttcctgaaccgctgccgcatgcatgccatctccggctggcaggccttctttcccatgcatttccaagccttccacccagctgtggccccaccacaagggcctgggcccccagagctgggccgtgacactggccgctttgatcgccaggcagccagcgaggcctgcttctacaactccgacaacggcagcccgtgggcgcctggcggcagctc

SEQ ID NO: 5 - skipped

SEQ ID NO: 6 - DNA, 1017 bp, Homo sapiens
catagtcagctcgttgatgtgcttgaggatctcattggagccgaacccggacagcatgagggtgtccctctccatgtccaggatggcgcaggccaccagcaggtgcagattggggccagggagccctgtccacagcacctcccacagccgaaggacatccgggaaggggaattccctcttgaaccagatgagcagccaccggaaacagaagcagagagagccggagtcctgggaatccaggaagtcgcagagcagggggtccagcaccctccggagcagcagcagtcgcccgagttgccgcttcatggtctcctggctctcttcaaagctcgagccgctcgagccgaattcggctcgagaaaccagcctgctcctggagcttccctggactcaacttcctaaaggcatgtgaggaaggggtagattccacaatctaatccgggggccatcagagtagagggagtagagaatggatgttgggtaggccatcaataaggtccattctgcgcagtatctcaactgccgttcaacaatcgcaagaggaaggtggagcaggtttcttcatcttacagttgagaaaacagagactcagaagggcttcttagttcatgtttcccttagcgcctcagtgattttttcatggtggcttaggccaaaagaaatatctaaccattcaatttataaataattaggtccccaacgaattaaatattatgtcctaccaacttattagctgcttgaaaaatataatacacataaataaaaaaatatatttttcagttctatttcagtgttaatgagaactacttactaaggagatgtatgcacctattgggacagtgtgcaagttcttcagctgggattgagggtgggcaatgctgcccctcaatttctgcttccaggtgggtggttccatatggtacttgagtttttatcagagggcctgggaaaaccccagtctcacaaaaatattgaaattatcagaagggttatagtggcaatcttatgttgaaagga

SEQ ID NO: 7 - DNA, 671 bp, Homo sapiens
cataagtatggttacaagctgcaggtgtctgcattcctcaatggcaatggcagtggtgagggcacacacctctcactgtacattcgtgtgctgcctggtgcctttgacaatctccttgagtggccctttgcccgccgtgtcaccttctccctgctggatcagagcgaccctgggctggctaaaccacagcacgtcactgagaccttccaccccgacccaaactggaagaatttccagaagccaggcacgtggcggggctccctggatgagagttctctgggctttggttatcccaagttcatctcccaccaggacattcgaaagcgaaactatgtgcgggatgatgcagtcttcatccgtgctgctgttgaactgccccggaagatcctcagctgagtgcaggtggggttcgaggggaaaggacgatggggcatgacctcagtcaggcactggctgaacttggagagggggccggacccccgtcagctgcttctgctgcctaggttctgttaccccatcctccctcccccagccaccaccctcaggtgcctccaattggtgcttcagccctggcccctgtggggaacaggtcttggggtcatgaagggctggaaacaagtgaccccagggcctgtctcccttcttgggtagggcagacatgcttgggtgcc

SEQ ID NO: 8-9 - skipped (2 entries)

SEQ ID NO: 10 - DNA, 870 bp, Homo sapiens
ccttcctcttctggttcagcgtggcctccctaatcaccctcttccacctcttcctcttcaagctcatctacaacgagtactgtgggcctggagccaagcccctcttcaggagtaaggaagatcccagtgtctgagtgaactaacagtcctgctttcagccaccatttgcacaagacacccagcactgaaagtcccgctgccaggagcaagggatcctttggaagcacccgccctttgtgccttgttgggggaaaccggtgacgcagaagtgagtgtggatacaccagagtttgcattggaaggaatgagtgtcacgtggggagggaaggggccagtggaccttttgtaagctttccactcaataaaatgaacctgtatggcaaatacttgaaatggaactcactccttccactttccccctttcttctgtcccaggaaatagatcatcttttgaaaagactcttgtctaggaaaagttgtgtccttttcctaatttaacgtgttctttcttaatgaagttttaatttatttttgttgagattttgctagatggcttttgcatcccctgtagatggtgagtgttggcggtgatgtccgtctcggcgttcggaggccccacggtcccgaggctgggccggggccccccagggtggctgtgctgctgcctgtaggagggtgcgggttgtgctgtcatcctcgggtttgcacgcccttttttaggagcctgtggacatctgtggttttgtactttggggcttcaggggaggtgtttaactttctagtgattgatgattgtcaggttttgaaataccaaagcttttttgttctgtttttaaataaatatctttcaaactttaaaaaaaaaaaaaa

SEQ ID NO: 11 - skipped

SEQ ID NO: 12 - DNA, 1311 bp, Homo sapiens
ttaaaagttatttatgtttaatgcttaaaagtctgaattcacaaacaatctaccattatagaagtactggtggtcaatacaatgcattagaactatgtacaacgcacagtttagtatcaaaatctttctacactgtagagttttacgaaactgttaatgacatcaaacactaagcacttaagacaccatttttttctgctaccacattaggaacgtcaatggacagtccatttcaacttgccgcatccatccatttctagtatgaaattaagtaattttctacttatacaataaagtatatctacacggttcttttgattttgatccatcgcagcaacggcactgtacatcagcctcgagccgattcggctcgagcttgcctgtgccctatgtcactgaggcctcacgtctcactgtgctgctgcctctagctgcggctgagcgtgacctggcccctggcttcttggaggcctttgccactgcagcactggagcctggtgatgctgcggcagccctgaccctgctgctactgtatgagccgcgccaggcccagcgcgtggcccatgcagatgtcttcgcacctgtcaaggcccatgtggcagagctggagcggcgtttccccggtgcccgggtgccatggctcagtgtgcagacagccgcaccctcaccactacgcctcatggatctactctccaagaagcacccgctggacacactgttcctgctggccgggccagacacggtgctcacgcctgacttcctgaaccgctgccgcatgcatgccatctccggctggcaggccttctttcccatgcatttccaagccttccacccagctgtggccccaccacaagggcctgggcccccagagctgggccgtgacactggccgctttgatcgccaggcagccagcgaggcctgcttctacaactccgactacgtggcagcccgtgggcgcctggcggcagcctcagaacaagaagaggagctgctggagagcctggatgtgtacgagctgttcctccacttctccagtctgcatgtgctgcgggcggtggagccggcgctgctgcagcgctaccgggcccagacgtgcagcgcgaggctcagtgaggacctgtaccaccgctgcctccagagcgtgcttgagggcctcggctcccgaacccagctggccatgctactctttgaacaggagcagggcaacagcacctgagcccaccctgtccccgtgggccgtggcatgggcaaaacccaccccacttctcccccaaaaccagagccaccgccagcctcgtgggcagggttggc

SEQ ID NO: 13 - DNA, 1008 bp, Homo sapiens
ccgccatccagcctgtcctttggaccacaccacccctccagcatggtcaccgccatgggttagagccctgctcgatgctcacagggcccccagcgagagtccctgcagtccctttcgacttgcatttttgcaggagcagtatcatgaagcctaaacgcgatggatatatgtttttgaaggcagaaagcaaaattatgtttgccactttgcaaaggagctcactgtggtgtctgtgttccaaccactgaatctggaccccatctgtgaataagccattctgactcatatcccctatttaacagggtctctagtgctgtgaaaaaaaaaaatgctgaacattgcatataacttatattgtaagaaatactgtacaatgactttattgcatctgggtagctgtaaggcatgaaggatgccaagaagtttaaggaatatgggagaaatagtgtggaaattaagaagaaactaggtctgatattcaaatggacaaactgccagttttgtttcctttcactggccacagttgtttgatgcattaaaagaaaggcgaatcatttgttcaaagctgttggcctctgcaaaggaaataccagttctgggcaatcagtgttaccgttcaccagttgccattgagggtttcagagagcctttttctaggcctacatgctttgtgaacaagtccctgtaattgttgtttgtatgtataattcaaagcaccaaaataagaaaagatgtagatttatttcatcatattatacagaccgaactgttgtataaatttatttactgctagtcttaagaactgctttctttcgtttgtttgtttcaatattttccttctctctcaatttttggttgaataaactagattacattcagttggcctaaggtggttgtgctcggagggtttcttgtttcttttccattttgtttttgggatgatatttattaaatagctcctaagagtccggcggcatctgtcttgtccctattcctgcagcctgtgct

SEQ ID NO: 14 - skipped

SEQ ID NO: 15 - DNA, 706 bp, Homo sapiens
gttgggggaaacccacgaggggacgcggccgaggagggtcgctgtccacccgggggcgtgggagtgaggactgcaagagggaggaaggcgggaacctaggaggcctgattaagatggtccatctactggtcttgtcaggtgcctggggcatgcaaatgtgggtgaccttcgtctcaggcttcctgcttttccgaagccttccccgacataccttcggactagtgcagagcaaactcttccccttctacttccacatctccatgggctgtgccttcatcaacctctgcatcttggcttcacagcatgcttgggctcagctcacattctgggaggccagccagctttacctgctgttcctgagccttacgctggccactgtcaacgcccgctggctggaaccccgcaccacagctgccatgtgggccctgcaaaccgtggagaaggagcgaggcctgggtggggaggtaccaggcagccaccagggtcccgatccctaccgccagctgcgagagaaggaccccaagtacagtgctctccgccagaatttcttccgctaccatgggctgtcctctctttgcaatctgggctgcgtcctgagcaatgggctctgtctcgctggccttgccctggaaataaggagcctctagcatgggccctgcatgctaataaatgcttcttcagaaatggcaaaaaaaaaaaaaaaaat

SEQ ID NO: 16 - skipped

SEQ ID NO: 17 - DNA, 450 bp, Homo sapiens
taatgtttaaaattcaattaggatttaccttactgctgtaaaatctggtctattttagtttcctctgggtagttagtgttgctaataagatggacgtaagtgtttttgaactggtgaattctgattgcttttagcccccagttttccaaataggggtgaattctgggtagagatagaacaatcaccaagttaccttgctccaaaaaagaaatttacgtatgggattgttttcaaagcgggaagttagctgtgtaaataacaacaattttatatatttaatctgggcttctccttatcttgaatgatataaaaatctactttctagattaatttagttccatataactttgtattgctttgactgtactgataataaagtttgaaagtgttaaatttaaaaaagaaaaaaagaggcaaaaggaaagacaagaaagggacccgggagggatc

SEQ ID NO: 18 - DNA, 418 bp, Homo sapiens
cgaagattcaaaagctccaaaaacctactgtagacatcgaagaaccaatatatacaatgggccaacaatccagtgtccgcaggctgaagaggagcgtcccctgtgaatccaacgaggccaacgaggccaatgaggccaacaagacgatgccggagaccccaactggggactcagacccgcaacctgctcctaaaaaaatgaaaacatctgagtcctcgaccatactagtggttcgctacaggaggaacgtgaaaagaacatctccagaggaactggtgaatgaccacgcccgagagaacagaatcaaccccgaccaaatggaggaggaggaattcatagaaatacgactaaagccttaaaagtagcaagaagctacatccctcaaacttcggcaatgaaaataaagtttgagaagctg

SEQ ID NO: 19 - DNA, 548 bp, Homo sapiens
gggcccgcatgccactttcctgccccagagtttaaacagaaagaagagtgtacaatccgtggccggagcctgatacggatgagcatccaggggacacctggaacctccccaactccatcaagaccctggtggacaacattcagagatatgtggaagatgggaagaaccagctgctcctggccttgctgaagtgcacagacacggagctgcagctgcgcagagacgcgatcttctgccaggccctggtggccgccgtgtgcaccttctccgagcagctgctggcggccctgggctaccgctacaacaacaatggcgagtacgaggagagcagccgcgacgccagccgcaagtggctggagcaggtggcggccacgggcgtcctgctgcactgccagtccctgctctcgcgagcgacagtgaaggagggacgggccatgctggaggtcatctgggtgacgctgtcagagctggacaatttgaccttctccctttaagggagctgggccgggaactattttgggcaagcaccaaatgtgttttaccgcatt

SEQ ID NO: 20 - DNA, 999 bp, Homo sapiens
caagatgacctctcccgtctacggcaccgacatgatggtgggcatcgggacgtcggatgtggacctggacaaataccgccacacgttctgcagcctgctgggcagggatgaggacagctggggcctctcctacacgggcctcctccaccacaagggcgacaagaccagcttctcgtcgcggttcggccagggctccatcattggcgtgcacctggacacctggcacggcacactcacctttttcaagaacaggaagtgtataggtgtggcagccaccaagctgcagaacaagagattctacccgatggtgtgctccacggcggcccggagcagcatgaaggtcacccgctcctgtgccagcgccacttccctccagtacctgtgctgccaccgcctgcgccactgcggccagactcgggagacacgctggagggtctgccgctgccgccgggcctcaagcaggtgctacacaacaagctgggctgggtcctgagcatgagttgcagccgccgcaaggtccagtgtccgatccccaggcagcgacctccgcccaccccagcagtcgcgagcctcggccctgccagaggaagcgctgccgccggacctgactgacttcccagtggaactgccttcttgggctgggacagccctttcctctgtcccttctttctctgtcccttccttccagccacactccagggcggagttggatgaggcccgtccggagggagccatctcttgctcccgaggctgggacagtcctttctgtgggggctctagggcccctctgctgctgtgctgggtggggaagcggctgccctgagccccaggtcttgtgggaggctgcgaggacgagagcctggctggagcccgcgttgctgttcccacagggcctcggtttttcctaacttgctctgcatgctgtcagcggctgccccgccgtcatagacttaaaggactgcaataaatgtagagttgatgtctaacaaacaaaaaaaa

SEQ ID NO: 21 - DNA, 311 bp, Homo sapiens
gggactttctctgaatgcagatttgttctgaatgttatcaccataggaggatttgtgacagggagtgccatgatcaaatttctgatttttgaatgttgtttgtaatagcaggtttgggatggtttgaaggagtagtagacaaaaggctgaaaaccctgtcggagagcagtagtccaggagagatgaaactgagtgatagaatggaaaagaaaggttttgagaggtgtgttggggaagggaggttggagaaataactttctgattgccaaacatgtgttcagacattctgctcatccttatctatttaattt

SEQ ID NO: 22 - DNA, 527 bp, Homo sapiens
gagacctcttgcaagatgcttctctgccgccataggctggaggttccccgggaaattttcccttccttcctagctgaggaagatccctcacttccgctcgccgcgccaccggtcccacctccccgccccccgctgggtcctagcgccggcccctgtttggcagggtccgggctccgtcggtgcgaggagccgacgccgacgccacggagtcagcacaagtctcatcagagaaaccccgttcaccaaggccatggaagtggaggctgcagaggcccggtccccagcccccggctacaagcgctcgggccgccgctacaagtgcctgtcctgtaccaagacatttccaaacgcgcccagggcagcgcgccacgctgccacacatgggccggcagactgctctgaagaggtggccgaggtgaagccaaagccagagacagaagctaaggcagaggaagccagtggggagaaggtgtcacggtccgacgccaagcctaggcccttatcggtgtccgctatgccccaaggcc

SEQ ID NO: 23 - DNA, 359 bp, Homo sapiens
gttccagacagaagaaatagcaagtgccgagaagctggcatcagaaaaacagaggggagatttgtgtggctgcagccgagggagaccaggaagatctgcatggtgggaaggacctgatgatacagaggtgagaaataagaaaggctgctgactttaccatctgaggccacacatctgctgaaatggagataattaacatcactagaaacagcaagatgacaatataatgtctaagtagtgacatgtttttgcacatttccagcccctttaaatatccacacacacaggaagcacaaaaggaagcacagagatccctgggagaaatgcccggccgccatcttgggtcatcgatgagcctc

SEQ ID NO: 24 - DNA, 1482 bp, Homo sapiens
cggggcgctccggggaggccaggacagctgatggttgtgccagaaacatctcaaggtagctggtccgcccccacttccccatctacctcttgtcctccccccaacaccaccaccaccctggctcccctccctcatgaccgcctggatcctcctgcctgtcagcctgtcagcgttctccatcactggcatatggactgtgcagcccaaggctgtgatgaaccaccatgtatgccctgtggagaactggtcctacaacgagtcctgccctcctgaccctgctgagcaagggggtcccaagacctgctgcaccctggacgatgtccccctcatcagcaagtgtggctcctatcccccagaaagctgcctcttcagcctcattggcaacatgggtgctttcatggtggccctgatctgcctcctgcgctacgggcagctcctggagcagagtcggcactcttgggttaacaccacggcactcatcacaggctgcaccaacgctgcgggcctcttggtggttggcaactttcaggtggatcatgccaggtctctgcactacgttggagctggcgtggccttccctgcggggctgctctttgtttgcctgcactgtgctctctcctaccaaggggccaccgccccgctggacctggctgtggcctatctgcgaagtgtgctggctgtcatcgcctttatcaccctggtcctcagtggagtcttctttgtccatgagagttctcagctgcaacatggggcagccctgtgtgagtgggtgtgtgtcatcgatatcctcattttctatggcaccttcagctacgagtttggggcagtctcctcagacacactggtggctgcactgcagcctacccctggccgggcctgcaagtcctccgggagcagcagcacctccacccacctcaactgtgcccccgagagcatcgctatgatctaaggtctggggagggtggctggcccggcctccacagcaccccaccccatatcttctttccatttatttcgtaccaaaaacaattttgagaaagtattctgttgggatctgggcttcctcacttctggagaagtggccatcccatgcccacctgtgccatggaggagtgggccctgccagctgccacagctgcatgacctgcttccccaccccacggtgtcgttttgtttttaaaggtcacctgtcctcactcacccagccagcccttcaggtgccttctactcccagtgccaaagccagaccactggggtttcctgctgcaggaattgggggctgggaacagcagaggggatagaagtctggtggaggtggagtgggcacgccttagcctacggaaaaacccatttctgggcccactgagctgcactgggattcttcactctgcccctcacttcctttagggcaaataacacagcagaaccacgtgggtattttagtacttttttttatattaaaaaaaaactaaattggcaaaa

SEQ ID NO: 25 - skipped

SEQ ID NO: 26 - DNA, 316 bp, Homo sapiens
gaaatccaaacaactgccattgatttattcatttatttcacaaatatttactgaacgcatccagcatgctctgtggggtgctgtgctggggctgggggtgccaggatgagaaacagccgtgtggctgtgctcttggcttcaccagccagacgagtgttgcctttgcaaggagaaaggactcacaaggcttacacatttgctgccctcagttttgccctttctcaaataaatctcacacatccaatctccttgtggcccattagggagtatataatgaaattaagtaaatgaggaattgcctaaaactaagggagtt

SEQ ID NO: 27 - DNA, 369 bp, Homo sapiens
cttcagccgcattcagcccctacttacctggggaccccggctggggcacgagagtaccaggggggtagggcccaaagggatcaggggaagcctctggcctggagggtatggggcacgcttccccaagggcggacccggcaggaggaagcccaggagctgggtcctgccgcccaggagctgggccctgccacccaggccgggctagggacatggcagggcctgggcatcctgacgctggacttgggcgacctgggaggcacagggaggggagagatgggcgggcccgccccagcgcagtgccggccacacccatgcaccgaagctcctccctgccacgccccaaggcggttgccggagcttaccgggggt

SEQ ID NO: 28 - skipped

SEQ ID NO: 29 - DNA, 591 bp, Homo sapiens
ggcgcatccgagccatggcccagcaggtgtttatgctggacacccagtgctcaccaaagacaccaaacaactttgaccacgctcagtcctgccagctcattattgagctgcctcctgatgaaaaaccaaatggacacaccaagaaaagcgtgtctttcagggaaattgtggtgagcctgctgtctcatcaggtgttactccagaacttatatgacatcttgttagaagagtttgtcaaaggcccctctcctggagaggaaaagacgatacaagtgccagaagccaagctggctggcttcctcagatacatctctatgcagaacttggcagtcatattcgacctgctgctggactcttataggactgccagggagtttgacaccagccccgggctgaagtgcctgctgaagaaagtgtctggcatcgggggcgccgccaacctctaccgccagtctgcgatgagctttaacatttatttccacgccctggtgtgtgctgttctcaccaatcaagaaaccatcacggccgagcaagtgaagaaggtcctttttgaggacgacgagagaagcacggattcttcccagcagtgtt

SEQ ID NO: 30 - skipped

SEQ ID NO: 31 - DNA, 1631 bp, Homo sapiens
gccccagcaggtctccgagcagccactgggacccgtctcagcacatcctggcctttgaaagtctgatatcctgagaggagggcaggttttagggccgcagttccagccagcgtccccagcctggcttccctgccatggactcagtagctcgtggggcttcttaccacccaccagccccgctggggtgcggcctggctgtgggcaaaggaggacttgcctggagatttgagagaagattccttctaccagggctgctgaggggccaggcctgcatcaggggctaggctctggctgggcccggaggctgagactaaggctttcgaccctggtgcctccatgtggatgctgcctcagacaaaggcagtgagccttccctgccaaagtgcccatcccatgggctcggcctcactggtcactgttagcccatgaacacgtgtgggcctcggtcacgtggctttgagggcagtctgaccaggctagaccacacgtgccgtgacagggggtgccattcccctcgcaggctctaatgtgcccacatgtagcctggcagtccaaagaccaagaatcaacttgcaaatctgccattaaactgctgtgcgacttcaggcatatcactgccttctctgggcttcagtgtccttttcatacctagaagtctgcggtctgaggctctttgggttcagacacactgttctaggcttctgtaggggaccttgtgatctgccgtgcccctcctccctgttcttttctgtcctccccaccccaccctcagaagctgcttgctctgcccccaggacaggagcttgacggatgaagtgcagccagccacccaggtgccatttccagtctgacttccagaaatgtgcaccatgtcctagagcacagacccattggctggagcctcctgggagggttcaaaccatcagctctatgagaaatgcccagaaaggctttgccgactccatccgtctgtggaggctgcctgcctccggggtgggatgggtggtttctcctccaattcagacccaagaggtagcccccgagggcatgtacctggtgggaagcagctcaggtacccttgggggttgcagggcccttacgcaggtatttctctctctctcctctctggggtgcgtgtgtgcgtgcgcgtgtgcgtgcctatgcttttctctgtgggcacatcaggatgcccctcggagagcatgtgcacgtgtccccacctgagcgagcgtgtgtgtgtgctcctctgcgtcccaggtttggacgtctagggtttggtgtgcctgtcttctgccctccctgagcccacagggtcagtcaatgtatcttctacgtgcctctccctctgccttctctcacagtgcccccggctccagagctcaggggtaggggttctcctgagggtgcaggggatccttctcatctcctggaccctccagggcactctggtccctattccccagctcctaggcagctgagccgggtcccttaggggaggtgaccaggagctttggtgcagggagctcttggtggggcaaagggctggacccctgccaggtctgtggacatggttatatgcccggggagaggggggtgcagggccccagggatggcccccaatcccacctctg

SEQ ID NO: 32 - skipped

SEQ ID NO: 33 - DNA, 844 bp, Homo sapiens
  modified_base (462): a, t, c, g, other or unknown
tatttttttttttactgatatattgtagtttaataaaacatagtttatacagttcattgaaaaagtattttaatacaaacaccacttatacacaaaaccaaatgttgatattcttgtttttaaaaattcttgatttctctaaaacactaagatgctatctcaatagagattgcttcacattttccagtttcttgatctgtgcatgtcacatgtaaagatccatcccttttcatagtaagaacagctaatatatcacgtaatccattttcttttttatctaaatcctggagtacaacctgtgcaaacttggtttcctctttggcagagttcttcccatcagactcatagagttcaaggcacactgaagatatgcttccaggggcttgcaatgtgtgttgtcttcgagctggcaaaggagtccctgatggaaacagcactgtgaatctactggctcctgattcngtccacacccttaactaaaatatctctggctgaacactctatcataagagagtcttccaccaacaggttttctttcccaataagaattcctgcttctatagctgcaccaatagggatcacttcatcaggagggatagaattgagaagctcaacagctgggaaaagatctttaatcagttgctgtagctttgggattcgagaagaccctccacaaaggacaaccttgttgatatcatctgctgtaaatccattttgatctaagagtcctctgattgcttctatacacttattaaaaagtggagaacaaagaagttcaaatcttgctctggacacattgcaatcaaaatcttgaccttcatataatgagtcaagaaaacagttg

SEQ ID NO: 34 - DNA, 1563 bp, Homo sapiens
aatgtagagaaagtggactcatcccccaagcgttgtctgcccccactctttcctccttgggtcccgccattcttttactgggcagtcgagggcattggaggggaagtgactgccctcagcctcactccctggggccatgaagaaaagctaaacagtctcatggcatctcagaataatgttgggtctcccaagaagaaaggtgtaagaataacgacatggctgattaggcgaggccaggatagggctaaggccaggattcctggctggcatccagtcaccccttctcccatccttccccctcttcttccacaagtccgcagccgagacactgtagtctcccagccacagtgatgagtgccctggagactccactgacctctagatgaaggcccctggccctggttcctgttaattaacctctgggtctttgagtcccccagcacaaacttctttcctgtaccctgcggcttggggtcacagggcatgccgggaagccacagctgaggggcgcagactgaagcagtgctccacctctccttctttagctcaggggttgctggtctgtggcaggcgccacgagtggcccctgtggctgttctcagtggcagtctcttaagttcccaccacaggcagctctttatcccctctccctacttgactctttctcttgcctgtgcttttggcctcaaacaggcctgctggtagcgctcagggcgtgaggctacactcctgccctgcctttcctgtcttcatggtctgccagggcataccttggggaggtggaccaaagacccaggactttttgcagtagccagtcctaccccccagttgtctttttaccaattcagggtgggagagaaaactgcagcaccccagcatgtgagttactcaggtgttgggggctagaagggacagtgcgtttaaacaacactcagagctctggccttaaacctgtggccccccaagtctaggagcctcatctcttcctggcagtcatgcgggcaggaggtcctgaaagggaaaacccattcagacaactgttccccaatctaccagccatctgcaggggtcagtgaccgtggccctctccctcctctagaatgtgccacttatgaagagtgccccatggggaaaaggagactcagctgtcccttggcagcttgtgccagtatcccagggcagaagtttccacaggagcctcttgcccttgcgcagagccactgtgagaggcggtgggagccaacacccttgggggagggggcagtactgctcggcacatcccagcatcaggtcagatcattgaaattaaaaaatgtgaattaagttcatatccaccttttggggaagcaggacaaaccaccaccccaccaagtgtgtgacttctccatatcccactgcagtttccattttttaaatgggaattttcaatcccctgtgcttgtctaacgtctgctttaaaaagtttgagaccctgttactgtttgaaaatgcatgcatgttacgatgaatctccaacctgaggaaaaaaataaaactcaaaaagctttgtgtaaaaaaaaaaaa

SEQ ID NO: 35 - skipped

SEQ ID NO: 36 - DNA, 1557 bp, Homo sapiens
ctacggtcagccagggtagccccatggccccagcccctacccccaagggggctacccacagggtccctacccccaagggggctacccacagggcccctacccacaagagggctacccacagggcccctacccccaagggggctacccccaggggccatatccccagagccccttcggcccccaacccctatggacagccacaggtcttcccaggacaagaccctgactcaccccagcatggaaactaccaggaggagggtcccccatcctactatgacaaccaggacttccctgccaccaactgggatgacaagagcatccgacaggccttcatccgcaaggtgttcctagtgctgaccttgcagctgtcggtgaccctgtccacggtgtctgtgttcacttttgttgcggaggtgaagggctttgtccgggagaatgtctggacctactatgtctcctatgctgtcttcttcatctctctcatcgtcctcagctgttgtggggacttccggcgaaagcacccctggaaccttgttgcactgcagtcggtcctgaccgccagcctgtcgtacatggtggggatgatcgccagcttctacaacaccgaggcagtcatcatggccgtgggcatcaccacagccgtctgcttcaccgtcgtcatcttctccatgcagacccgctacgacttcacctcatgcatgggcgtgctcctggtgagcatggtggtgctcttcatcttcgccattctctgcatcttcatccggaaccgcatcctggagatcgtgtacgcctcactgggcgctctgctcttcacctgcttcctcgcagtggacacccagctgctgctggggaacaagcagctgtccctgagcccagaagagtatgtgtttgctgcgctgaacctgtacacagacatcatcaacatcttcctgtacatcctcaccatcattggccgcgccaaggagtagccgagctccagctcgctgtgcccgctcaggtggcacggctggcctggaccctgcccctggcacggcagtgcagctgtacttcccctctctcttgtccccaggcacagcctagggaaaaggatgcctctctccaaccctcctgtatgtacactgcagatacttccatttggacccgctgtggccacagcatggcccctttagtcctcccgcccccgccaaggggcaccaaggccacgtttccgtgccacctcctgtctactcattgttgcatgagccctgtctgccagcccaccccagggactgggggcagcaccaggtcccggggagagggattgagccaagaggtgagggtgcacgtcttccctcctgtcccagctccccagcctggcgtagagcacccctcccctcccccccacccccctggagtgctgccctctggggacatgcggagtgggggtcttatccctgtgctgagccctgagggcagagaggatggcatgtttcaggggagggggaagccttcctctcaatttgttgtcagtgaaattccaataaatgggatttgctctctgccaaaaaaaaaaaaaaa

SEQ ID NO: 37 - DNA, 1381 bp, Homo sapiens
agtactctgacacctccaccctctactttattagaattggaaggcaaatttttgtccaaaaacctacagacaagtactttgagagaatttccaatataatattagacataatgataattttttccatactcagaatgaaaaactggatattacgtttttgttttggggtttttttgtacaaatttagctaatagctacaggctgagagaattgtaacatagcatgacaaattttgtgttgacttgaaaggaatcacaccattattccttagaagtaattacatgtgttctaacacatttgagacagggttggactcccatttctcatccgagaaattacttaacccttcctggcgctgtacagtcatcttttattctatttcctctttgctgtttgtagtagagacattttgaatgaaacttggcactgcttgattcaaaactgtggaaaccagatctgtttagtctcctgtttgtatgcgtttgctaatggtagctaaataaccagtttttgttgtaaatgcaccaattctgaaggcactttatgtactacatggaggtcatatctggttttgtttttatttttttatcatgaacattaaatgtgatgatgatttcttttccctgcacacatctttccggtgcaatatctatcaattgtgaatctggctgctggtgtataaaaacctggatgtaaagctgagcctacagacctgtcctcaccaactgttttgtgatttctactcaactacaaagatttatttaatgtactcttaatctaactgagttttgttaccaatgacctgttgcatgcttcaataccgtgtactgcctgagttgtgcctcttgtgtgctagattaaaagtgagacagagacttgacttgatcctctgagctcaagctattgagctggtagtggcagaggactgagggtacctgcacagtttgattcttttccacgtgtaagtctccattgcagaattgtcgtgctttgagaaaacacctgaggcagtgtgggagttgaacgaccctgctgtcctttttaacctgtgttgtcctagaccctgtcggggcagtcaggggacactagagatttgatctcatgcgagtcatcaataggacaaaaaagttgtggtttggggaggtctgtttgttacataaaaaggacctttcggtgtaagaaattgccgtttttaccctgccctggctggcatgtgagaagccatggaaggttgtggttgtaaatgagttgtctaaaggggtgcagaggcctgaggtttctaaaagaaggtagatttctacagagctgagtgttggttcctttttcttattggttgaaaattacctggtagtgatcagaaaacttagatgctatgtaacta

SEQ ID NO: 38 - skipped

SEQ ID NO: 39 - DNA, 615 bp, Homo sapiens
gttgctccagtgtttgaaataagaagactcatgtttatctctggagaccttgctggcagtgctagccaggaaacagagtgaccaagggacaagaagggacttgcctaaagccacccagcaactcagcagcagaaccaagatgggccccaggctcctccatatggcccagggcttaccaccctatcacacgtggccttgtctagacccagtcctgagcaggggagaggctcttgagacctgatgccctcctacccacatggttctcccactgccctgtctgctctgctgctacagaggggcagggcctcccccagcccacgcttaggaatgcttggcctctggcaggcaggcagctgtacccaagctggtgggcagggggctggaaggcaccaggcctcaggaggagccccatagtcccgcctgcagcctgtaaccatcggctgggccctgcaaggcccacactcacgccctgtgggtgatggtcacggtgggtgggtgggggctgaccccagcttccaggggactgtcactgtggacgccaaaatggcataactgagataaggtgaataagtgacaaataaagccagttttttacaaggtaaaaaaaaaaaaaac

SEQ ID NO: 40 - DNA, 834 bp, Homo sapiens
ccggaaccagaactggaatccgcccttaccgcttgctgccaaaacagtgggggctgaactgacctctcccctttgggagagaaaaactgtctgggagcttgacaaaggcatgcaggagagaacaggagcagccacagccaggagggagagccttccccaagcaaacaatccagagcagctgtgcaaacaacggtgcataaatgaggcctcctggaccatgaagcgagtcctgagctgcgtcccggagcccacggtggtcatggctgccagagcgctctgcatgctggggctggtcctggccttgctgtcctccagctctgctgaggagtacgtgggcctgtctgcaaaccagtgtgccgtgccagccaaggacagggtggactgcggctacccccatgtcacccccaaggagtgcaacaaccggggctgctgctttgactccaggatccctggagtgccttggtgtttcaagcccctgcaggaagcagaatgcaccttctgaggcacctccagctgcccccggccgggggatgcgaggctcggagcacccttgcccggctgtgattgctgccaggcactgttcatctcagcttttctgtccctttgctcccggcaagcgcttctgctgaaagttcatatctggagcctgatgtcttaacgaataaaggtcccatgctccacccgaggacagttcttcgtgcctgagactttctgaggttgtgctttatttctgctgcgtcgtggacagcgggagggtgtcaggggagagtctgcccaggcctcaagggcaggaaaagactccctaaggagctgcagtgcatgcaaggatatttt

SEQ ID NO: 41-43 - skipped (3 entries)

SEQ ID NO: 44 - DNA, 997 bp, Homo sapiens
tgcctctctgcaatagacagctactgtcaatacatgctgtaatttgacattctgggtcacagatataaggtatttaaaatctatttatgctttatagagaaaccagacattaaaacttcatgcactacttatttcgaattactgtaccttatccaaatttacacctagctattaggatcttcaacccaggtaacaggaataattctgtggtttcatttttctgtaaacaactgaaagaataattagatcatattctagtatgttctgaaatatctttaagactgatcttaaaaactaacttctaagatgatttcatcttctcatagtatagagtttactttgtacacgtttgaaaccaactactgtagaagatgaggaatctattgtaattttttgctttattttcatctgccagtggacttatttgaaattttcactttagtcaaattattttttgtattagtttttgatgcagacataaaaatagcaatcattttaaattgtcaaaatttccagattactggtaaaaattatttgaaaacaaacttatgggtaataaaggctagtcagaaccctataccataaagtgtagttaccatacagattaatatgtagcaaaaatgtatgcttgatatttctcaactgtgttaatttttctgctgtattccagctgaccaaaacaatattaagaatgcatctttataaatgggtgctaattgataatggaaataatttagtaatggactatacaggatgttaataatgaagccatatgtttatgtctggatttaaaaattttaaacaatcatttactatgtcatttttctttaccttgaagaacataaactgttatttcacttctacaaatcagcaagatattatttatggcaagaaatattccattgaaatattgtgctgtaacatgggaaagtgtaaatgtttttcatggtttctatcaatgtgaaataaaatttaattctgaaaaaaaaaagaaaga

SEQ ID NO: 45 - DNA, 548 bp, Homo sapiens
caaacagctggggcccaagatcgtcatcgtgagcaagatgatgaaggacgtgttcttcttcctcttcttcctcgcgtgtggctggtagcctatggcgtggccacggaggggctcctgaggccacgggacagtgacttcccaagtatcctgcgccgcgtcttctaccgtccctacctgcagatcttcgggcagattccccaggaggacatggacgtggccctcatggagcacagcaactgctcgtcggagcccggcttctgggcacaccctcctggggcccaggcgggcacctgcgtctcccagtatgccaactggctggtggtgctgctcctcgtcatcttcctgctcgtggccaacatcctgctggtcaacttgctcattgccatgttcagttacacattcggcaaagtacagggcaacagcgatctctactggaaggcgcaggttaccgcctcatccgggaattccactctcggcccgcgctggccccgccctttatcgtcatctcccacttgcgcctcctgctcaggcaattgtgcaggcgagcc

SEQ ID NO: 46 - DNA, 1448 bp, Homo sapiens
gtatttcgtgagaagttcaacaccaaaactggaacatagttctccttcaagtgttggcgacacgggcgcttcctgattctggaatataactttgtgtaaattaacagccacctatagaagagtccatctgctgtgaaggagagacagagaactctgggttccgtcgtcctgtccacgtgctgtaccaagtgctggtgccagcctgttacctgttctcactgaaaagtctggctaatgctcttgtgtagtcacttctgattctgacaatcaatcaatcaatggcctagagcactgactgttaacacaaacgtcactagcaaagtagcaacagctttaagtctaaatacaaagctgttctgtgtgagaattttttaaaaggctacttgtataataacccttgtcatttttaatgtacaaaacgctattaagtggcttagaatttgaacatttgtggtctttatttactttgcttcgtgtgtgggcaaagcaacatcttccctaaatatatattaccaagaaaagcaagaagcagattaggtttttgacaaaacaaacaggccaaaagggggctgacctggagcagagcatggtgagaggcaaggcatgagagggcaagtttgttgtggacagatctgtgcctactttattactggagtaaaagaaaacaaagttcattgatgtcgaaggatatatacagtgttagaaattaggactgtttagaaaaacaggaatacaatggttgtttttatcatagtgtacacatttagcttgtggtaaatgactcacaaaactgattttaaaatcaagttaatgtgaattttgaaaattactacttaatcctaattcacaataacaatggcattaaggtttgacttgagttggttcttagtattatttatggtaaataggctcttaccacttgcaaataactggccacatcattaatgactgacttcccagtaaggctctctaaggggtaagtaggaggatccacaggatttgagatgctaaggccccagagatcgtttgatccaaccctcttattttcagaggggaaaatggggcctagaagttacagagcatctagctggtgcgctggcacccctggcctcacacagactcccgagtagctgggactacaggcacacagtcactgaagcaggccctgtttgcaattcacgttgccacctccaacttaaacattcttcatatgtgatgtccttagtcactaaggttaaactttcccacccagaaaaggcaacttagataaaatcttagagtactttcatactcttctaagtcctcttccagcctcactttgagtcctccttggggttgataggaattttctcttgctttctcaataaagtctctattcatctcatgtttaatttgtacgcatagaattgctgagaaataaaatgttctgttcaacttaaaaaaaaaaa

SEQ ID NO: 47 - DNA, 1163 bp, Homo sapiens
ctcgggcaagtgcttcagcctggtggagagtcggaagagatgcacgggtcctggaagagacctcacactgatccccagggtccaagcatctcttccgactctcgggcaagtgcttcagcctggtggagtccacgtgagtgcagggtgggtgcgagggtgggctggggcgcagctgcggacccccctcatgccatctgtgtccccaggtacaagtatgagttctgcccgttccacaacgtgacccagcacgagcagaccttccgctggaacgcctacagtgggatcctcggcatctggcacgagtgggagatcgccaacaacaccttcacgggcatgtggatgagggacggtgacgcctgccgttcccggagccggcagagcaaggtggagctggcgtgtggaaaaagcaaccggctggcccatgtgtccgagccgagcacctgcgtctacgcgctgacgttcgagacccccctcgtctgccacccccacgccttgctagtgtacccaaccctgccagaggccctgcagcggcagtgggaccaggtagagcaggacctggccgatgagctgatcaccccccagggccatgagaagttgctgaggacactttttgaggatgctggctacttaaagaccccagaagaaaatgaacccacccagctggagggaggtcctgacagcttggggtttgagaccctggaaaactgcaggaaggctcataaagaactctcaaaggagatcaaaaggctgaaaggtttgctcacccagcacggcatcccctacacgaggcccacagaaacttccaacttggagcacttgggccacgagacgcccagagccaagtctccagagcagctgcggggtgacccaggactgcgtgggagtttgtgaccttgtggtgggagagcagaggtggacgcggccgagagccctacagagaagctggctggtaggacccgcaggaaccagctgaccaggcttgtgctcagagaagcagacaaaacaaagattcaaggttttaattaattcccatactgataaaaataactccatgaattcctgtaaaccattgcataaatgtctatagtgtaaaaaaatttaaacaagtgttaactttaaacagttcgtctacaagtaaatgattataaatactaaaaaaaaaa

SEQ ID NO: 48 - DNA, 906 bp, Homo sapiens
cgaggaccggccttgcgagcggcgacactataaaatggcgcgtgctgcaacccgcgcccgcttcggagagagaaatgctggggtgcagcttcaagcttaggaccacccaccatgcctatccaggtgctgaagggcctgaccatcactcattaagaacagaggaggctgcctgttactcctggtgttgcatccctccagacactctgctgtttcctgcctaggcgtggctgcagccatggctaggaaagcgctgccacccacccacctgggccagagctggttctgctcctgctgcagggacactgagctggctatctcggcgcttcgggcaagaactgcaacaggctctcctgggtcctgcaggtgtacagccgggcccctgccttgtgcctcagctctcgagagctgctgctgccgggtgacctgatccaacctgataaggtgccatcttcagctaccactgcaaggccctgagggcaacagcagcacggcactgcccacccggctgctgatggcctggtgccagctgggagtcctcccggcacttcgaggccactgagccacccttccagccccagcccaccatggacaggggtatccagcttcctcctcaacctcgtcctctgcccctgagccagtgacgcccaaggacatgcctgttacccaggtcctgtacaagcactagctggtaaagggcatgacagtgctggaggccgtcttggagatccaggccatcactggcagcaggctgctctccatggtgccagggcccgccaggccaccaggctcatgctgggacccaacccagtgcacaaggacttggctgctgagccacacacccaggagaaggtggataagtgggctaccaagggcttcctgcaggctaggggaggagccaccccgctt

SEQ ID NO: 49 - DNA, 1222 bp, Homo sapiens
tattagatatggcagaaaaaccatttccactatgcaaagttcttttagacgtcagtgaaaatcaactctcatacctcatggtctctctttaattgaccaaaaccttccatttttctctaaatacaaagcgatctgtgttctgagcaacctttccccgaacacacagcttcagtgcagcacgctgacctgagtatccaccatgtgccaggcacagtgctgggcacacgaggcaccaaggtccgggccacctgcccgcagcaaggcccagctgaggtggtggagggagcccctgaggtcaggggccgtttcggttcagggtggcaggtgtccagcactggggtatggcgtcgaggcttccatggggtgggggaggccagcttccttctgacaggatgggcgcatacagtgcctggtgtgatttgtgcacaacccgtgttccaggtgcacatcctcccaaggagacacccagacccttccagcacgggccggccaagttgctgcggcggaggcagcatttcagctgtgaggaaggtcattggattcatgtgttttatctgtaaaaatggttgtcttaacttcttaacctcatattggtaagtgattgataaaaattggttggtgtttcatgacatgtggacttcttttgaaatagcaagtcaaatgtagtgaccaaattgtggaagagatttctgtcaaataggaaatgtgtaagttcgtctaaaagctgatggttatgtaagttgctcaggcactcagatgacagcagattctgggttctgggagtgttctgtgcctcttacatgccctggaggcctcatggtctcagtgctgaggcggcacacctgtagcacacctgcgtaatgtgcggtctgggccagtcacaaggaattgtgttgtctaagccaaagggggaagctgactgtgatttaccaaaaaaaattctgtaattcaaaccaaaatgtctgcggaatcaccagtttgatactctctgtaatcagaacagtgggcagtgcctgggtgaacgtgtctagcagccactgtgcgggatcgctgtaacaggagtggaatgtacatatttatttacttttctaactgctccaacagccaaatgccttttttatgaccattgtattcagttcattaccaaagaaatgtttgcactttgtaatgatgcctttcagttcaaataaatgggtcacattttcaaatggaaaaaaaaaaa

SEQ ID NO: 50 - DNA, 649 bp, Homo sapiens
agatattatggatggtgaagggaatggtatagaattggagagattatcttactgaacacctgtagtcccagctttctctggaagtggtggtatttgagcaggatgtgcacaaggcaattgaaatgcccataattagtttctcagctttgaatacactataaactcagtggctgaaggaggaaattttagaaggaagctactaaaagatctaatttgaaaaactacaaaagcattaactaaaaaagtttattttccttttgtctgggcagtagtgaaaataactactcacaacattcactatgtttgcgagggattaacacaaataaaagatgcctttttacttaaacaccaagacagagaacttgcccaatactgagaagcaacttccactagagagggaactgttaaatgttttcaacccagttcatctggtggatgtttttgcaggttactctgagaattttgcttatgaaaaatcattatttttagtgtagttcacaataatgtattgaacatacttctaatcaaaggtgctatgtccttgtgtatggtactaaatgtgtcctgtgtacttttgcacaactgagaatcctgcagcttggtttaatgagtgtgttcatgaaataaataatggaggaattgtcaaaaa

SEQ ID NO: 51 - DNA, 1226 bp, Homo sapiens
cccaactccacccagggatcctggtgtacgggctgacctgttatgcttttctgcccttcggccctttggggagccacggcgggaggtggagatccaccggcgatatgtggcccagtcggtccagctctttattctctacttcttcaacctggccgtgctttccacttacctgccccaggataccctcaaactgctccctctgctcactggtctctttgccgtctcccggctgatctactggctgacctttgccgtgggccgctccttccgaggcttcggctacggcctgacgtttctgccactgctgtcgatgctgatgtggaacctctactacatgttcgtggtggagccggagcgcatgctcactgccaccgagagccgcctggactacccggaccacgcccgctcggcctccgactacaggccccgcccctggggctgagcctctccgccctcgccctcggagtagggggtagcggcttgggtctgacacatctttgaaccttgtggccaggcctggacttcgcccccaggcctaggaccgcggtgggtggaaccctgctactgccccaacagggactccaatcaatcggagttctccccttgccggagctgcccttcacctttggggcccgagacagtcataagggatggacttagttttcttgcagggaaaaaggtggacagccgtgtttcttaaggatgctgagggcatggggccaggaccaggggagaggcacagctccttcctgagcagcctctcaccactgccacaaggctccctaatgctggtctctgctccactccccggcttcccgtgaggcaggaggcagagccacagccaaggccctgaccacttctgtgccagttgtctaagcagagcgcctcagggacgctggaaatgccttaaggatagaggctgggcatcacatcaaatgggactgtggtgtttggtgaaaaccttcctgaggatctggattcaggaccctccatgactggcctatttactgtttacagctggccagtgcagagctgctgctcttttacctttttaggcccctgtaacttcccacctttaaactgcccagaaggcatgcctctcccacaggaagaggggagcagacagggaaatctgcctaccaagaggggtgtgtgtgtctttgtgcccacacgtggtggctggggagtgcctggatggtgcggtggttgatgttaacctagtgtgt

SEQ ID NO: 52 - DNA, 1036 bp, Homo sapiens
aactgatattgggaagtttggttgaggttcaagtgtgactccttccagagccacaggtaggggagtgtgaagttgagggggaggaaagctggaaggactctgccttgggagattcccagctctgctttccagcgcttggtggaatctgggctggggaaagacggcaccgggaaactctgcttccccattgtttccatctgatcagctgtggtgtgaggacttctcagacaaaggcaaggcctcgtgcccctgcccagcccattcatggagccctgggccttcttggcttccatagatcctaagctcttgactgtagtttagccagacttgttttgctatcttataagcagttcagaattagggaatgctggttttgaagagcaaaggacaggtagtctagagagggtcgtctggcctgcttgctgggtctttgtaacccagcacttcctcttgccctcctggctttatgtttatggggagaggactcaatagctccaccccttctggcaccagatggggcttggttagtttgcaataagcaccttgcagaggttaaagccagcgggtccctagtcttaggcccagcctgcttgtgtgggctctggcctggcctggtggctggcccagggggcagcagtgcttagagcttctgcagggcttctcttgtttacacagctgcatcagacaatgccatttctccccaccacggaaccttccatctaagatttcttccagggaatgccagcaatcaggcagcacccagctgtgggggcagtggggtgggggagacccacattgatgactttttttttttcttttaatgaagaaacaccaaagaaagctgtggaaaggacctgccccacatgaaaaggataagccaagatggctgtaaacacagagcatttgagctgccactcttggagcacattgatttttcaaaagccagctctgtcaggaaaggaggtgctgttatgagcagctcttccagtgggcaaagaggacgcccataatttcttccattgctagctcatctgtg

SEQ ID NO: 53 - DNA, 758 bp, Homo sapiens
cggcacgagctcccatgggacctaggtttcagcggccgctgcgatgaccaaaataaaggcagatcccgacgggcccgaggctcaggcggaggcgtgttccggggagcgcacctaccaggagctgctggtcaaccagaaccccatcgcgcagccctggcttctcgccgcctcacgcggaagctctacaaatgcatcaagaaagcggtgaagcagaagcagattcggcgcggggtgaaagaggttcagaaatttgtcaacaaaggagaaaaagggatcatggttttggcaggagacacactgcccattgaggtatactgccatctcccagtcatgtgtgaggaccgaaatttgccctatgtctatatcccctctaagacggacctgggtgcagccgcaggtccaagcgccccacctgtgtgataatggtcaagccccatgaggagtaccaggaggcttacgatgagtgcctggaggaggtgcagtccctgcccctacccctatgaggggctccggtagcacctgggcacctgccgctggaagctattgggctggcagcaggacgactggctgtcctcctgcccacccacactgacggcatcttcccagttccccaaggcacgccttcttcccaggcagctctaacagccctttcatgaaggtaatgctagtcttctgtccatcagtgccatttcctgtagaactaaaggctgttccaagaatgtggggtggggaaagtaaatgctaagactaaaaaaaaa

SEQ ID NO: 54-56 - skipped (3 entries)

SEQ ID NO: 57 - protein, 244 aa, Homo sapiens
MVVEVPRWSNAKMEIATKDPLNPIKQDVKKGKLRYVANLFPYKGYIWNYGAIPQTWEDPGHNDKHTGCCGDNDPIDVCEIGSKVCARGEIIGVKVLGILAMIDEGETDWKVIAINVDDPDAANYNDINDVKRLKPGYLEATVDWFRRYKVPDGKPENEFAFNAEFKDKDFAIDIIKSTHDHWKALVTKKTNGKGISCMNTTLSESPFKCDPDAARAIVDALPPPCESACTVPTDVDKWFHHQKN

SEQ ID NO: 58 - protein, 233 aa, Homo sapiens
MEYTLDLQLEALTPQGGRRPLTRRVQLLRPLSRVEILPVPYVTEASRLTVLLPLAAAERDLAPGFLEAFATAALEPGDAAAALTLLLLYEPRQAQRVAHADVFAPVKAHVAELERRFPGARVPWLSVQTAAPSPLRLMDLLSKKHPLDTLFLLAGPDTVLTPDFLNRCRMHAISGWQAFFPMHFQAFHPAVAPPQGPGPPELGRDTGRFDRQAASEACFYNSDNGSPWAPGGS

SEQ ID NO: 59 - protein, 56 aa, Homo sapiens [sequence omitted]

SEQ ID NO: 60 - protein, 130 aa, Homo sapiens
VQGSSRSRLVSRAEFGSSGSSFEESQETMKRQLGRLLLLRRVLDPLLCDFLDSQDSGSLCFCFRWLLIWFKREFPFPDVLRLWEVLWTGLPGPNLHLLVACAILDMERDTLMLSGFGSNEILKHINELTM

SEQ ID NO: 61 - protein, 131 aa, Homo sapiens
HKYGYKLQVSAFLNGNGSGEGTHLSLYIRVLPGAFDNLLEWPFARRVTFSLLDQSDPGLAKPQHVTETFHPDPNWKNFQKPGTWRGSLDESSLGFGYPKFISHQDIRKRNYVRDDAVFIRAAVELPRKILS

SEQ ID NO: 62-63 - skipped (2 entries)

SEQ ID NO: 64 - protein, 78 aa, Homo sapiens [sequence omitted]

SEQ ID NO: 65 - skipped

SEQ ID NO: 66 - protein, 314 aa, Homo sapiens
VIFYLYNKVYLHGSFDFDPSQQRHCTSASSRFGSSLPVPYVTEASRLTVLLPLAAAERDLAPGFLEAFATAALEPGDAAAALTLLLLYEPRQAQRVAHADVFAPVKAHVAELERRFPGARVPWLSVQTAAPSPLRLMDLLSKKHPLDTLFLLAGPDTVLTPDFLNRCRMHAISGWQAFFPMHFQAFHPAVAPPQGPGPPELGRDTGRFDRQAASEACFYNSDYVAARGRLAAASEQEEELLESLDVYELFLHFSSLHVLRAVEPALLQRYRAQTCSARLSEDLYHRCLQSVLEGLGSRTQLAMLLFEQEQGNST

SEQ ID NO: 67 - protein, 178 aa, Homo sapiens
KQASLAAWRSKRPVSRPSSGGPGPCGGATAGWKAWKCMGKKACQPEMACMRQRFRKSGVSTVSGPASRNSVSSGCFLESRSMRRSGEGAAVCTLSHGTRAPGKRRSSSATWALTGAKTSAWATRWAWRGSYSSSRVRAAAASPGSSAAVAKASKKPGARSRSAAARGSSTVRREASVT

SEQ ID NO: 68 - protein, 20 aa, Homo sapiens
PPSSLSFGPHHPSSMVTAMG

SEQ ID NO: 69 - skipped

SEQ ID NO: 70 - protein, 21 aa, Homo sapiens
LGETHEGTRPRRVAVHPGAWE

SEQ ID NO: 71 - protein, 198 aa, Homo sapiens
DGPSTGLVRCLGHANVGDLRLRLPAFPKPSPTYLRTSAEQTLPLLLPHLHGLCLHQPLHLGFTACLGSAHILGGQPALPAVPEPYAGHCQRPLAGTPHHSCHVGPANRGEGARPGWGGTRQPPGSRSLPPAAREGPQVQCSPPEFLPLPWAVLSLQSGLRPEQWALSRWPCPGNKEPLAWALHANKCFFRNGKKKKKN

SEQ ID NO: 72 - skipped

SEQ ID NO: 73 - protein, 30 aa, Homo sapiens
NLVYFSFLWVVSVANKMDVSVFELVNSDCF

SEQ ID NO: 74 - protein, 42 aa, Homo sapiens
NNHQVTLLQKRNLRMGLFSKREVSCVNNNNFIYLIWASPYLE

SEQ ID NO: 75 - protein, 108 aa, Homo sapiens
KIQKLQKPTVDIEEPIYTMGQQSSVRRLKRSVPCESNEANEANEANKTMPETPTGDSDPQPAPKKMKTSESSTILVVRYRRNVKRTSPEERVNDHARENRINPDQWRR

SEQ ID NO: 76 - protein, 62 aa, Homo sapiens
GPACHFPAPEFKQKEECTIRGRSLIRMSIQGTPGTSPTPSRPWWTTFRDMWKMGRTSCSWPC

SEQ ID NO: 77 - protein, 76 aa, Homo sapiens [sequence omitted]

SEQ ID NO: 78 - protein, 36 aa, Homo sapiens
QDDLSRLRHRHDGGHRDVGCGPGQIPPHVLQPAGQG

SEQ ID NO: 79 - protein, 167 aa, Homo sapiens [sequence omitted]

SEQ ID NO: 80 - protein, 96 aa, Homo sapiens
AECLNTCLAIRKLFLQPPFPNTPLKTFLFHSITQFHLSWTTALRQGFQPFVYYSFKPSQTCYYKQHSKIRNLIMALPVTNPPMVITFRTNLHSEKV

SEQ ID NO: 81 - protein, 151 aa, Homo sapiens
LRKIPHFRSPRHRSHLPAPRWVLAPAPVWQGPGSVGARSRRRRHGVSTSLIRETPFTKAMEVEAAEARSPAPGYKRSGRRYKCLSCTKTFPNAPRAARHAATHGPADCSEEVAEVKPKPETEAKAEEASGEKVSRSDAKPRPLSVSAMPQG

SEQ ID NO: 82 - protein, 105 aa, Homo sapiens
RLIDDPRWRPGISPRDLCASFCASCVCGYLKGLEMCKNMSLLRHYIVILLFLVMLIISISADVWPQMVKSAAFLISHLCIIRSFPPCRSSWSPSAAATQISPLFF

SEQ ID NO: 83 - protein, 304 aa, Homo sapiens
WLCQKHLKVAGPPPLPHLPLVLPPTPPPPWLPSLMTAWILLPVSLSAFSITGIWTVQPKAVMNHHVCPVENWSYNESCPPDPAEQGGPKTCCTLDDVPLISKCGSYPPESCLFSLIGNMGAFMVALICLLRYGQLLEQSRHSWVNTTALITGCTNAAGLLVGNFQVDHARSLHYVGAGVAFPAGLLFVCLHCALSYQGATAPLDLAVAYLRSVLAVIAFITLVLSGVFFVHESSQLQHGAALCEWVCVIDILIFYGTFSYEFGAVSSDTLVAALQPTPGRACKSSGSSSTSTHLNCAPESIAMI

SEQ ID NO: 84 - skipped

SEQ ID NO: 85 - protein, 52 aa, Homo sapiens
MCKPCESFLLAKATLVWLVKPRAQPHGCFSSWHPQPQHSTPQSMLDAFSKYL

SEQ ID NO: 86 - protein, 112 aa, Homo sapiens [sequence omitted]

SEQ ID NO: 87 - protein, 192 aa, Homo sapiens
MAQQVFMLDTQCSPKTPNNFDHAQSCQLIIELPPDEKPNGHTKKSVSFREIVVSLLSHQVLLQNLYDILLEEFVKGPSPGEEKTIQVPEAKLAGFLRYISMQNLAVIFDLLLDSYRTAREFDTSPGLKCLLKKVSGIGGAANLYRQSAMSFNIYFHALVCAVLTNQETITAEQVKKVLFEDDERSTDSSQQC

SEQ ID NO: 88 - skipped

SEQ ID NO: 89 - protein, 34 aa, Homo sapiens
PCPQTWQGSSPLPHQELPAPKLLVTSPKGPGSAA

SEQ ID NO: 90 - protein, 100 aa, Homo sapiens
TLPGGSSQWVCALGHGAHFWKSDWKWHLGGWLHFIRQAPVLGAEQAASEGGVGRTEKNREEGHGRSQGPLQKPRTVCLNPKSLRPQTSRYEKDTEAQRRQ

SEQ ID NO: 91 - skipped

SEQ ID NO: 92 - protein, 116 aa, Homo sapiens [sequence omitted]

SEQ ID NO: 93 - skipped

SEQ ID NO: 94 - protein, 321 aa, Homo sapiens
TVSQGSPMAPAPTPKGATHRVPTPKGATHRAPTHKRATHRAPTPKGATPRGHIPRAPSAPNPYGQPQVFPGQDPDSPQHGNYQEEGPPSYYDNQDFPATNWDDKSIRQAFIRKVFLVLTLQLSVTLSTVSVFTFVAEVKGFVRENVWTYYVSYAVFFISLIVLSCCGDFRRKHPWNLVALQSVLTASLSYMVGMIASFYNTEAVIMAVGITTAVCFTVVIFSMQTRYDFTSCMGVLLVSMVVLFIFAILCIFIRNRILEIVYASLGALLFTCFLAVDTQLLLGNKQLSLSPEEYVFAALNLYTDIINIFLYILTIIGRAKE

SEQ ID NO: 95 - protein, 62 aa, Homo sapiens
YSDTSTLYFIRIGRQIFVQKPTDKYFERISNIILDIMIIFSILRMKNWILRFCFGVFLYKFS

SEQ ID NO: 96 - skipped

SEQ ID NO: 97 - protein, 80 aa, Homo sapiens
MAARALCMLGLVLALLSSSSAEEYVGLSANQCAVPAKDRVDCGYPHVTPKECNNRGCCFDSRIPGVPWCFKPLQEAECTF

SEQ ID NO: 98-100 - skipped (3 entries)

SEQ ID NO: 101 - protein, 61 aa, Homo sapiens
MSGFKNFKQSFTMSFFFTLKNINCYFTSTNQQDIIYGKKYSIEILCCNMGKCKCFSWFLSM

SEQ ID NO: 102 - protein, 40 aa, Homo sapiens
MHYLFRITVPYPNLHLAIRIFNPGNRNNSVVSFFCKQLKE

SEQ ID NO: 103 - protein, 37 aa, Homo sapiens
MARNIPLKYCAVTWESVNVFHGFYQCEIKFNSEKKKK

SEQ ID NO: 104 - protein, 31 aa, Homo sapiens
MKIKQKITIDSSSSTVVGFKRVQSKLYTMRR

SEQ ID NO: 105 - protein, 29 aa, Homo sapiens
MIAIFMSASKTNTKNNLTKVKISNKSTGR

SEQ ID NO: 106 - protein, 113 aa, Homo sapiens
MDVALMEHSNCSSEPGFWAHPPGAQAGTCVSQYANWLVVLLLVIFLLVANILLVNLLIAMFSYTFGKVQGNSDLYWKAQVTASSGNSTLGPRWPRPLSSSPTCASCSGNCAGE

SEQ ID NO: 107 - protein, 81 aa, Homo sapiens [sequence omitted]

SEQ ID NO: 108 - protein, 71 aa, Homo sapiens
MAMSKLTSRMLATSRKMTRSSTTSQLAYWETQVPAWAPGGCAQKPGSDEQLLCSMRATSMSSWGICPKICR

SEQ ID NO: 109 - protein, 41 aa, Homo sapiens
MLRPQRSFDPTLLFSEGKMGPRSYRASSWCAGTPGLTQTPE

SEQ ID NO: 110 - protein, 52 aa, Homo sapiens [sequence omitted]

SEQ ID NO: 111 - protein, 58 aa, Homo sapiens [sequence omitted]

SEQ ID NO: 112 - protein, 296 aa, Homo sapiens [sequence omitted]

SEQ ID NO: 113 - protein, 122 aa, Homo sapiens
IFLHYRHLCNGLQEFMELFLSVWELIKTLNLCFVCFSEHKPGQLVPAGPTSQLLCRALGRVHLCSPTTRSQTPTQSWVTPQLLWRLGSGRLVAQVLQVGSFCGPRVGDAVLGEQTFQPFDLL

SEQ ID NO: 114 - protein, 126 aa, Homo sapiens [sequence omitted]

SEQ ID NO: 115 - protein, 63 aa, Homo sapiens
MARKALPPTHLGQSWFCSCCRDTELAISALRARTATGSPGSCRCTAGPLPCASALESCCCRVT

SEQ ID NO: 116 - protein, 66 aa, Homo sapiens
MTVLEAVLEIQAITGSRLLSMVPGPARPPGSCWDPTQCTRTWLLSHTPRRRWISGLPRASCRLGEE

SEQ ID NO: 117 - protein, 48 aa, Homo sapiens
MLGCSFKLRTTHHAYPGAEGPDHHSLRTEEAACYSWCCIPPDTLLFPA

SEQ ID NO: 118 - protein, 66 aa, Homo sapiens
MSLGVTGSGAEDEVEEEAGYPCPWWAGAGRVAQWPRSAGRTPSWHQAISSRVGSAVLLLPSGPCSG

SEQ ID NO: 119 - protein, 46 aa, Homo sapiens
MAAATPRQETAECLEGCNTRSNRQPPLFLMSDGQALQHLDRHGGWS

SEQ ID NO: 120 - protein, 47 aa, Homo sapiens [sequence omitted]

SEQ ID NO: 121 - protein, 43 aa, Homo sapiens
MVGWGWKGGSVASKCREDSQLAPGHQQPGGQCRAAVALRALQW

SEQ ID NO: 122 - protein, 158 aa, Homo sapiens
IQSDLCSEQPFPEHTASVQHADLSIHHVPGTVLGTRGTKVRATCPQQGPAEVVEGAPEVRGRFGSGWQVSSTGVWRRGFHGVGEASFLLTGWAHTVPGVICAQPVFQVHILPRRHPDPSSTGRPSCCGGGSISAVRKVIGFMCFICKNGCLNFLTSYW

SEQ ID NO: 123 - protein, 62 aa, Homo sapiens
MCQAQCWAHEAPRSGPPARSKAQLRWWREPLRSGAVSVQGGRCPALGYGVEASMGWGRPASF

SEQ ID NO: 124 - protein, 72 aa, Homo sapiens
MASRLPWGGGGQLPSDRMGAYSAWCDLCTTRVPGAHPPKETPRPFQHGPAKLLRRRQHFSCEEGHWIHVFYL

SEQ ID NO: 125 - protein, 142 aa, Homo sapiens [sequence omitted]

SEQ ID NO: 126 - protein, 26 aa, Homo sapiens
MFSTQFIWWMFLQVTLRILLMKNHYF

SEQ ID NO: 127 - protein, 54 aa, Homo sapiens [sequence omitted]

SEQ ID NO: 128 - protein, 28 aa, Homo sapiens
MGISIALCTSCSNTTTSRESWDYRCSVR

SEQ ID NO: 129 - protein, 28 aa, Homo sapiens
MNTLIKPSCRILSCAKVHRTHLVPYTRT

SEQ ID NO: 130 - protein, 152 aa, Homo sapiens
MLFCPSALWGATAGGGDPPAICGPVGPALYSLLLQPGRAFHLPAPGYPQTAPSAHWSLCRLPADLLADLCRGPLLPRLRLRPDVSATAVDADVEPLLHVRGGAGAHAHCHREPPGLPGPRPLGLRLQAPPLGLSLSALALGVGGSGLGLTHL

SEQ ID NO: 131 - protein, 72 aa, Homo sapiens [sequence omitted]

SEQ ID NO: 132 - protein, 56 aa, Homo sapiens
MDLVFLQGKRWTAVFLKDAEGMGPGPGERHSSFLSSLSPLPQGSLMLVSAPLPGFP

SEQ ID NO: 133 - protein, 42 aa, Homo sapiens
MPAIRQHPAVGAVGWGRPTLMTFFFSFNEETPKKAVERTCPT

SEQ ID NO: 134 - protein, 30 aa, Homo sapiens
MAVNTEHLSCHSWSTLIFQKPALSGKEVLL

SEQ ID NO: 135 - protein, 48 aa, Homo sapiens
MKRISQDGCKHRAFELPLLEHIDFSKASSVRKGGAVMSSSSSGQRGRP

SEQ ID NO: 136 - protein, 116 aa, Homo sapiens
MLCVYSHLGLSFSCGAGPFHSFLWCFFIKRKKKSHQCGSPPPHCPHSWVLPDCWHSLEEILDGRFRGGEKWHCLMQLCKQEKPCRSSKHCCPLGQPPGQARAHTSRLGLRLGTRWL

SEQ ID NO: 137 - protein, 97 aa, Homo sapiens
MEAKKAQGSMNGLGRGTRPCLCLRSPHTTADQMETMGKQSFPVPSFPSPDSTKRWKAELGISQGRVLPAFLPLNFTLPYLWLWKESHLNLNQTSQYQ

SEQ ID NO: 138 - protein, 139 aa, Homo sapiens
RHELPWDLGFSGRCDDQNKGRSRRARGSGGGVFRGAHLPGAAGQPEPHRAALASRRLTRKLYKCIKKAVKQKQIRRGVKEVQKFVNKGEKGIMVLAGDTLPIEVYCHLPVMCEDRNLPYVYIPSKTDLGAAAGPSAPPV

SEQ ID NO: 139 - protein, 30 aa, Homo sapiens
WSSPMRSTRRLTMSAWRRCSPCPYPYEGLR

SEQ ID NO: 140 - protein, 41 aa, Homo sapiens
HLGTCRWKLLGWQQDDWLSSCPPTLTASSQFPKARLLPRQL

SEQ ID NO: 141 - protein, 31 aa, Homo sapiens
QPFHEGNASLLSISAISCRTKGCSKNVGWGK

SEQ ID NO: 142 - DNA, 1663 bp, Homo sapiens
ggtgggtggagccaggcttggcgggctgtgcgtgctcgcggtgggcggtggcggcggctgcctcgcgaaggttcgagatccgtcgcgtgcgggaggcgggccgcgatcttgcgcagggtcggtgtgggcgcaggctgcagcgccgcgactcgtgcgggtaggcgtctgcgctcggtttgagggctcggcgcggggtttcctgttccttcttctgcgcggctgcagctcgggacttcggcctgacccagcccccatggcttcagaagagctacagaaagatctagaagaggtaaaggtgttgctggaaaaggctactaggaaaagagtacgtgatgcccttacagctgaaaaatccaagattgagacagaaatcaagaacaagatgcaacagaaatcacagaagaaagcagaacttcttgataatgaaaaaccagctgctgtggttgctcccattacaacgggctatacggtgaaaatcagtaattatggatgggatcagtcagataagtttgtgaaaatctacattaccttaactggagttcatcaagttcccactgagaatgtgcaggtgcatttcacagagaggtcatttgatcttttggtaaagaatctaaatgggaagagttactccatgattgtgaacaatctcttgaaacccatctctgtggaaggcagttcaaaaaaagtcaagactgatacagttcttatattgtgtagaaagaaagtggaaaacacaaggtgggattacctgacccaggttgaaaaggagtgcaaagaaaaagagaagccctcctatgacactgaaacagatcctagtgagggattgatgaatgttctaaagaaaatttatgaagatggagacgatgatatgaagcgaaccattaataaagcctgggtggaatcaagagagaagcaagccaaaggagacacggaattttgagactttaaagtcgttttgggaactgtgatgtgatgtggaaatactgatgtttccagtaagggaatattggtgagctgcatatataaatttgacagatagctatttacatagccttctaagtaaaggcaatgaattctccatttcctactggaggatttatttaaataaaatatgcttattaaacactcctgcaaagatggttttattagtaccctggtcattttgttcaaggaagggttatattgcattctcacgtgaaatataaaaagcaagtcttgcccaataaaaacgctacattgtgtgtattttttgttcagctaagaattggaaaagtatttgcttgccttttaagttactgacatcagcttccaccagtgtaaaaattgagtaaaacctgaagttttgcataaaatgcaaatcggtgcctgtgcttgaaggttgctgtagagcatctgaccccttattaccaccttaagcaatgtatatgccatgcattaccatgcactaattcaatcacaggtgtttctatctagatttaaatatatttgtcaatgaatgtggaatagaaaatctaaacatgacaataatagacatatctttgtatggtaccagttagttttgccgtggatcagatggtttataaaagtaataaccataaagcaaaaaataatttgaaagcccgtctattcctatgctcaataaagttaagtttttcttcattaa

SEQ ID NO: 143 - protein, 312 aa, Homo sapiens
VGGARLGGLCVLAVGGGGGCLAKVRDPSRAGGGPRSCAGSVWAQAAAPRLVRVGVCARFEGSARGFLFLLLRGCSSGLRPDPAPMASEELQKDLEEVKVLLEKATRKRVRDALTAEKSKIETEIKNKMQQKSQKKAELLDNEKPAAVVAPITTGYTVKISNYGWDQSDKFVKIYITLTGVHQVPTENVQVHFTERSFDLLVKNLNGKSYSMIVNNLLKPISVEGSSKKVKTDTVLILCRKKVENTRWDYLTQVEKECKEKEKPSYDTETDPSEGLMNVLKKIYEDGDDDMKRTINKAWVESREKQAKGDTEF

SEQ ID NO: 144 - DNA, 2105 bp, Homo sapiens
caggacttcatcggcggagagcccacccccggccgctactgccacggaggctttggggtgctgctgtcgcgcatgctgctgcaacaactgcgcccccacctggaaggctgccgcaacgacatcgtcagtgcgcgccctgacgagtggctgggtcgctgcattctcgatgccaccggggtgggctgcactggtgaccacgagggggtgcactatagccatctggagctgagccctggggagccagtgcaggagggggaccctcatttccgaagtgccctgacagcccaccctgtgcgtgaccctgtgcacatgtaccagctgcacaaagctttcgcccgagctgaactggaacgcacgtaccaggagatccaggagttacagtgggagatccagaataccagccatctggccgttgatggggaccgggcagctgcttggcccgtgggtattccagcaccatcccgcccggcctcccgctttgaggtgctgcgctgggactacttcacggagcagcatgctttctcctgcgccgatggctcaccccgctgcccactgcgtggggctgaccgggctgatgtggccgatgttctggggacagctctagaggagctgaaccgccgctaccacccggccttgcggctccagaagcagcagctggtgaatggctaccgacgctttgatccggcccggggtatggaatacacgctggacttgcagctggaggcactgaccccccagggaggccgccggcccctcactcgccgagtgcagctgctccggccgctgagccgcgtggagatcttgcctgtgccctatgtcactgaggcctcacgtctcactgtgctgctgcctctagctgcggctgagcgtgacctggcccctggcttcttggaggcctttgccactgcagcactggagcctggtgatgctgcggcagccctgaccctgctgctactgtatgagccgcgccaggcccagcgcgtggcccatgcagatgtcttcgcacctgtcaaggcccatgtggcagagctggagcggcgtttccccggtgcccgggtgccatggctcagtgtgcagacagccgcaccctcaccactacgcctcatggatctactctccaagaagcacccgctggacacactgttcctgctggccgggccagacacggtgctcacgcctgacttcctgaaccgctgccgcatgcatgccatctccggctggcaggccttctttcccatgcatttccaagccttccacccagctgtggccccaccacaagggcctgggcccccagagctgggccgtgacactggccgctttgatcgccaggcagccagcgaggcctgcttctacaactccgactacgtggcagcccgtgggcgcctggcggcagcctcagaacaagaagaggagctgctggagagcctggatgtgtacgagctgttcctccacttctccagtctgcatgtgctgcgggcggtggagccggcgctgctgcagcgctaccgggcccagacgtgcagcgcgaggctcagtgaggacctgtaccaccgctgcctccagagcgtgcttgagggcctcggctcccgaacccagctggccatgctactctttgaacaggagcagggcaacagcacctgaccccaccctgtccccgtgggccgtggcatggccacaccccaccccacttctcccccaaaaccagagccacctgccagcctcgctgggcagggctggccgtagccagaccccaagctggcccactggtcccctctctggctctgtgggtccctgggctctggacaagcactgggggacgtgcccccagagccacccacttctcatcccaaacccagtttccctgccccctgacgctgctgattcgggctgtggcctccacgtatttatgcagtacagtctgcctgacgccagccctgcctctgggccctgggggctgggctgtagaagagttgttggggaaggagggagctgaggagggggcatctcccaacttctcccttttggaccctgccgaagctccctgcctttaataaactggccaagtgtggaaaaaaaaaaaaaaaaaaaaaaaaaaaaaaaaaaaa

SEQ ID NO: 145 - DNA, 1125 bp, Homo sapiens
cgggcggcccaccccagatctcctgcacgttgtggggccctcggcgcacgcggttagctggcggtgcagggcctcggcgcgggtcagcacgtcctccacgctcagcttcatagtcagctcgttgatgtgcttgaggatctcattggagccgaagccggacagcatgagggtgtccctctccatgtccaggatggcgcaggccaccagcaggtgcagattggggccagggagccctgtccacagcacctcccacagccgaaggacatccgggaaggggaattccctcttgaaccagatgagcagccaccggaaacagaagcagagagagccggagtcctgggaatccaggaagtcgcagagcagggggtccagcaccctccggagcagcagcagtcgcccgagttgccgcttcatggtctcctggctctcttcaaagctcgagccgctcgagccgaattcggctcgagaaaccagcctgctcctggagcttccctggactcaacttcctaaaggcatgtgaggaaggggtagattccacaatctaatccgggggccatcagagtagagggagtagagaatggatgttgggtaggccatcaataaggtccattctgcgcagtatctcaactgccgttcaacaatcgcaagaggaaggtggagcaggtttcttcatcttacagttgagaaaacagagactcagaagggcttcttagttcatgtttcccttagcgcctcagtgattttttcatggtggcttaggccaaaagaaatatctaaccattcaatttataaataattaggtccccaacgaattaaatattatgtcctaccaacttattagctgcttgaaaaatataatacacataaataaaaaaatatatttttcagttctatttcagtgttaatgagaactacttactaaggagatgtatgcacctattgggacagtgtgcaagttcttcagctgggattgagggtgggcaatgctgcccctcaatttctgcttccaggtgggtggttccatatggtacttgagtttttatcagagggcctgggaaaaccccagtctcacaaaaatattgaaattatcagaagggttatagtggcaatcttatgttgaaagga

SEQ ID NO: 146 - DNA, 1490 bp, Homo sapiens
tcggcactggggggcaggggtcgatcggctcaactatgaagatcactgcttcagcgggcacgccaccatgcacgccgagaacctgtggccggggcggctgtcctccgtccagcagatcctgcagctctctgacctgtggaggctgaccctccagaagcgtggctgcaaggggctggtgaaggtgggtgccccaggcatcctgcaggggatggtgctcagctttggggggctgcagttcacagagaaccacctccagttccaggccgaccccgacgtgctgcacaacagctatgcattgcatggcatccgctacaagaacgaccatatcaacctggccgtgctggcggatgccgagggcaagccctacctacacgtgtccgtggagtcccgtggccagcctgtcaagatctatgcctgcaaggcaggctgcctggacgagccagtggagctgacctcggcgcccacgggccacaccttctcggtcatggtgacacagcccatcacgccactgctctacatctccaccgacctcacacacctgcaggacctgcggcacacgctgcacctcaaggccatcctggcccatgatgagcacatggcccagcaggaccccgggctgcccttcctcttctggttcagcgtggcctccctaatcaccctcttccacctcttcctcttcaagctcatctacaacgagtactgtgggcctggagccaagcccctcttcaggagtaaggaagatcccagtgtctgagtgaactaacagtcctgctttcagccaccatttgcacaagacacccagcactgaaagtcccgctgccaggagcaagggatcctttggaagcacccgccctttgtgccttgttgggggaaaccggtgacgcagaagtgagtgtggatacaccagagtttgcattggaaggaatgagtgtcacgtggggagggaaggggccagtggaccttttgtaagctttccactcaataaaatgaacctgtatggcaaatacttgaaatggaactcactccttccactttccccctttcttctgtcccaggaaatagatcatcttttgaaaagactcttgtctaggaaaagttgtgtccttttcctaatttaacgtgttctttcttaatgaagttttaatttatttttgttgagattttgctagatggcttttgcatcccctgtagatggtgagtgttggcggtgatgtccgtctcggcgttcggaggccccacggtcccgaggctgggccggggccccccagggtggctgtgctgctgcctgtaggagggtgcgggttgtgctgtcatcctcgggtttgcacgcccttttttaggagcctgtggacatctgtggttttgtactttggggcttcaggggaggtgtttaactttctagtgattgatgattgtcaggttttgaaataccaaagcttttttgttctgtttttaaataaatatctttcaaactttcaaaaaaaaaaaaa

SEQ ID NO: 147 - DNA, 1692 bp, Homo sapiens
tttttttttttactagaagttatttatgtttaatgcttaaaagtctgaattcacaaacaatctaccattatagaagtactggtggtcaatacaatgcattagaactatgtacaacgcacagtttagtatcaaaatctttctacactgtagagttttacgaaactgttaatgacatcaaacactaagcacttaagacaccatttttttctgctaccacattaggaacgtcaatggacagtccatttcaacttgcagcatccatccatttctagtatgaaattaagtaattttctacttatacaataaagtatatctacacggttcttttgattttgatccatcgcagcaacggcactgtacatcagcctcgagccgattcggctcgagcttgcctgtgccctatgtcactgaggcctcacgtctcactgtgctgctgcctctagctgcggctgagcgtgacctggcccctggcttcttggaggcctttgccactgcagcactggagcctggtgatgctgcggcagccctgaccctgctgctactgtatgagccgcgccaggcccagcgcgtggcccatgcagatgtcttcgcacctgtcaaggcccatgtggcagagctggagcggcgtttccccggtgcccgggtgccatggctcagtgtgcagacagccgcaccctcaccactacgcctcatggatctactctccaagaagcacccgctggacacactgttcctgctggccgggccagacacggtgctcacgcctgacttcctgaaccgctgccgcatgcatgccatctccggctggcaggccttctttcccatgcatttccaagccttccacccagctgtggccccaccacaagggcctgggcccccagagctgggccgtgacactggccgctttgatcgccaggcagccagcgaggcctgcttctacaactccgactacgtggcagcccgtgggcgcctggcggcagcctcagaacaagaagaggagctgctggagagcctggatgtgtacgagctgttcctccacttctccagtctgcatgtgctgcgggcggtggagccggcgctgctgcagcgctaccgggcccagacgtgcagcgcgaggctcagtgaggacctgtaccaccgctgcctccagagcgtgcttgagggcctcggctcccgaacccagctggccatgctactctttgaacaggagcagggcaacagcacctgaccccaccctgtccccgtgggccgtggcatggccacaccccaccccacttctcccccaaaaccagagccacctgccagcctcgctgggcagggctggccgtagccagaccccaagctggcccactggtcccctctctggctctgtgggtccctgggctctggacaagcactgggggacgtgcccccagagccacccacttctcatcccaaacccagtttccctgccccctgacgctgctgattcgggctgtggcctccacgtatttatgcagtacagtctgcctgacgccagccctgcctctgggccctgggggctgggctgtagaagagttgttggggaaggagggagctgaggagggggcatctcccaacttctcccttttggaccctgccgaagctccctgcctttaataaactggccaagtgtggaaaaaaaaaaaaaaaaaaaaaaaaaaaaaaaaaaaa

SEQ ID NO: 148 - DNA, 866 bp, Homo sapiens
ctacgacccgattggcttcgggctcagctgggaggcgggacgaattattggttgggggaaacccacgaggggacgcggccgaggagggtcgctgtccacccgggggcgtgggagtgaggtaccagattcagcccatttggccccgacgcctctgttctcggaatccgggtgctgcggattgaggtcccggttcctaacggactgcaagatggaggaaggcgggaacctaggaggcctgattaagatggtccatctactggtcttgtcaggtgcctggggcatgcaaatgtgggtgaccttcgtctcaggcttcctgcttttccgaagccttccccgacataccttcggactagtgcagagcaaactcttccccttctacttccacatctccatgggctgtgccttcatcaacctctgcatcttggcttcacagcatgcttgggctcagctcacattctgggaggccagccagctttacctgctgttcctgagccttacgctggccactgtcaacgcccgctggctggaaccccgcaccacagctgccatgtgggccctgcaaaccgtggagaaggagcgaggcctgggtggggaggtaccaggcagccaccagggtcccgatccctaccgccagctgcgagagaaggaccccaagtacagtgctctccgccagaatttcttccgctaccatgggctgtcctctctttgcaatctgggctgcgtcctgagcaatgggctctgtctcgctggccttgccctggaaataaggagcctctagcatgggccctgcatgctaataaatgcttcttcagaaaaaaaaaaaaaaaaaaaaggggagaaagaaaaaggaaaaggcgagaggagcg

SEQ ID NO: 149 - DNA, 992 bp, Homo sapiens
atttctacttctctccaagcgtccaatttataataacatgtgcatcttgttttccctgccaaactaaatttcgtgagggggtacatactgcacagttttttttgtcacatactgcttatgacacatgaatatgcagagcttgtcaagctctttaattaagtttaaaatgctaattgagtgaatcaaaacttaaccattatggttaggctaaaaatgtcagcttgtgtttatatagtgcttacctcagtattggaaatgccatgagtttagtatcagaaggacattattactagtgcattttaaagtgataccagtcatagttgcaaaagaaagtacacaatgggaaatggaagagaaatgtagggaatcaaaacaactagttttttcctttataacggaagttttataattcatcttttatgtaagtgtaattctcattaaaaataccctaaagcttaaagtttgcaaggctgcccagcctaacccacaacagtttgatgctgccccctagcgtttgattcccttcaccttttgctaaaataaggtaatgtttaaattacaattagatttacttactgctgtaaatctggtctattttagtttcctctgggtagttagtgttgctaataagatggacgtaagtgtttttgaactggtgaattctgattgcttttagcccccagttttccaaataggggtgaattctgggtagagatagaacaatcaccaagttaccttgctccaaaaaagaaatttacgtatgggattgttttcaaagcgggaagttagctgtgtaaataacaacaattttatatatttaatctgggcttctccttatcttgaatgatataaaaatctactttctagattaatttagttccatataactttgtattgctttgactgtactgataataaagtttgaaagtgttaaatttaaaaaagaaaaaaagaggcaaaaggaaagacaagaaagggacccgggagggatc

SEQ ID NO: 150 - DNA, 1640 bp, Homo sapiens
ggtccggggcggaaagtgggtcagggccgggccggcggacgcgcagggggggctgcagattctttccaccatggccagacgcccccggaacagcagggcctggcacttcgtcctgagtgcagcccgccgagacgcagatgcccgggccgtggctctagcaggctccactaactggggctacgactctgatgggcagcacagcgactcggactccgaccccgagtactccacgctgccgccatccatccccagtgcggtgcccgtgaccggcgagtccttctgtgactgtgctgggcagagcgaggcctccttctgtagcagcctgcactcggcccaccggggcagggactgccgctgcggagaggaagacgagtgtgagcgtggggcccagcccctgctgggactgcactgcgggcgggcctgggggtgggcagggcagagccccatggagctctcagggctgccccagcctgggcctctcttgcagatttcgactgggtctgggatgacttaaataagtcatcagccaccctgctgagctgtgacaaccgtaaggtcagcttccacatggagtacagctgcggcacagcggccatccggggcaccaaggagctgggggagggccagcacttctgggagatcaagatgacctctcccgtctacggcaccgacatgatggtgggcatcgggacgtcggatgtggacctggacaaataccgccacacgttctgcagcctgctgggcagggatgaggacagctggggcctctcctacacgggcctcctccaccacaagggcgacaagaccagcttctcgtcgcggttcggccagggctccatcattggcgtgcacctggacacctggcacggcacactcacctttttcaagaacaggaagtgtataggtgtggcagccaccaagctgcagaacaagagattctacccgatggtgtgctccacggcggcccggagcagcatgaaggtcacccgctcctgtgccagcgccacttccctccagtacctgtgctgccaccgcctgcgccagtgcggccagactcgggagacacgctggagggtctgccgctgccgccgggcctcaagcaggtgctacacaacaagctgggctgggtcctgagcatgagttgcagccgccgcaaggtccagtgtccgatccccaggcagcgacctccgcccaccccagcagtcgcgagcctcggccctgccagaggaagcgctgccgccggacctgactgacttcccagtggaactgccttcttgggctgggacagccctttcctctgtcccttctttctctgtcccttccttccagccacactccagggcggagttggatgaggcccgtccggagggagccatctcttgctcccgaggctgggacagtcctttctgtgggggctctagggcccctctgctgctgtgctgggtggggaagcggctgccctgagccccaggtcttgtgggaggctgcgaggacgagagcctggctggagcccgcgttgctgttcccacagggcctcggtttttcctaacttgctctgcatgctgtcagcggctgccccgccgtcatagacttaaaggactgcaataaatgtagagttgatgtctaacacccaaaaaaaa

SEQ ID NO: 151 - DNA, 974 bp, Homo sapiens
gcacgctatgctagccacacttccggcaagagcagctagaatcgttccggtgcatattgaataggcgcagaaagggagaaaaagattctacagccctggccacagtactttggtgacacttttcgtggggctctctggaggacttttcccaaggcagatggagaaaacttcgtgaaacccactccttgctattaaaggaaatgttgtggaatataattggacttaggttttgcagagcttgagcatggcctttttgtcctcccaccttctggttcttgaagacattgccggtgacctggccccagactaacacaaggcgggcgtataccgtcagcctgcctggcgtccccttgcctcagcacacacagagacctcttgcaagatgcttctctgccgccataggctggaggttccccgggaacttttcccttccttcctagctgaggaagatccctcacttccgctcgccgcgccaccggtcccacctccccgccccccgctgggtcctagcgccggcccctgtttggcagggtccgggctccgtcggtgcgaggagccgacgccgacgccacggagtcagcacaagtctcatcagagaaaccccgttcaccaaggccatggaagtggaggctgcagaggcccggtccccagcccccggctacaagcgctcgggccgccgctacaagtgcctgtcctgtaccaagacatttccaaacgcgcccaggggagcgcgccacgctgccacacatgggccggcagactgctctgaagaggtggccgaggtgaagccaaagccagagacagaagctaaggcagaggaagccagtggggagaaggtgtcagcgtccggcgccaagcctaggccctatcggtgtccgctatgccccaaggcctacaagacggcacccgacgtgcgcagccaccggcgcagccacacgggggagaagccctttccgtgccccgagtgcggccgc

SEQ ID NO: 152 - DNA, 1059 bp, Homo sapiens
gtgggatgaccgcgtgtataggactctcaggcttttatcctagatcaccactggattgctgacagatagaggacgtgggaccgtgactatcacccctaatctgcagtggatttggctctcggcactcccaggctgggagctggatacctgccctggcagcatgactcagactgcatcacagtcacagactcgcctctgctcctgtggtccagtggccggacaccccctgggatggctcaaaggagtcaggacttggaagtggggacatcagggtagctgaaggaaatccacacacccagagcatctcggagttcagactctcagacctgaagtaggcgcccccgggactgggctaggagttggacggaatggaggatggaggacagcgagaagaaaggaagagaaatgcaaagtgtgggcagccgccaagagtgaaaatagagggaagtgtcatgcaagtgctggacagaaggcggcaggtgggacgagccccacagccccctcctcaaaaacgaccacctccaggactcagtgatccctggggggcaggctctgccagccctcggccacacgtggctccggcacccatggtcccagtgccttggatggagacggccagttctggcggccagatgtggtgctctggaatccagtcccatttccttcctggccacgcctgtccagcggcctcttcagccgcattcagcccctacttacctggggaccccggctggggcacgagagtaccaggggggtagggcccaaagggatcaggggaagcctctggcctggagggtatggggcacgcttccccaagggcggacccggcaggaggaagcccaggagctgggtcctgccgcccaggagctgggccctgccacccaggccgggctagggacatggcagggcctgggcatcctgacgctggacttgggcgacctgggaggcacagggaggggagagatgggcgggcccgccccagcgcagtgccggccacacccatgcaccgaagctcctccctgccacgccccaaggcggttgccggagcttaccgggggt

SEQ ID NO: 153 - DNA, 2003 bp, Homo sapiens
gagagatctgaaataacctttcccagtgggcagggttgccagggttgaggggacagcacataccacccccacccaacctgttcgaggggccctgcatggcacgggatgagtccctgccctgtgcagctgcctggcagtggctgggacaaggatcttgcagccagcacagaggcctcttcaaaggcctctccctcttggcactccaggcaaggcaggtgcccgcttccccaacacctccaggcagtgaccctagggcatgccccagcaggtctccgagcagccactgggacccgtctcagcacatcctggcctttgaaagtctgatatcctgagaggagggcaggttttagggccgcagttccagccagcgtccccagcctggcttccctgccatggactcagtagctcgtggggcttcttaccacccaccagccccgctggggtgcggcctggctgtgggcaaaggaggacttgcctggagatttgagagaagattccttctaccagggctgctgaggggccaggcctgcatcaggggctaggctctggctgggcccggaggctgagactaaggctttcgaccctggtgcctccatgtggatgctgcctcagacaaaggcagtgagccttccctgccaaagtgcccatcccatgggctcggcctcactggtcactgttagcccatgaacacgtgtgggcctcggtcacgtggctttgagggcagtctgaccaggctagaccacacgtgccgtgacagggggtgccattcccctcgcaggctctaatgtgcccacatgtagcctggcagtccaaagaccaagaatcaacttgcaaatctgccattaaactgctgtgcgacttcaggcatatcactgccttctctgggcttcagtgtccttttcatacctagaagtctgcggtctgaggctctttgggttcagacacactgttctaggcttctgtaggggaccttgtgatctgccgtgcccctcctccctgttcttttctgtcctccccaccccaccctcagaagctgcttgctctgcccccaggacaggagcttgacggatgaagtgcagccagccacccaggtgccatttccagtctgacttccagaaatgtgcaccatgtcctagagcacagacccattggctggagcctcctgggagggttcaaaccatcagctctatgagaaatgcccagaaaggctttgccgactccatccgtctgtggaggctgcctgcctccggggtgggatgggtggtttctcctccaattcagacccaagaggtagcccccgagggcatgtacctggtgggaagcagctcaggtacccttgggggttgcagggcccttacgcaggtatttctctctctctcctctctggggtgcgtgtgtgcgtgcgcgtgtgcgtgcctatgcttttctctgtgggcacatcaggatgcccctcggagagcatgtgcacgtgtccccacctgagcgagcgtgtgtgtgtgctcctctgcgtcccaggtttggacgtctagggtttggtgtgcctgtcttctgccctccctgagcccacagggtcagtcaatgtatcttctacgtgcctctccctctgccttctctcacagtgcccccggctccagagctcaggggtaggggttctcctgagggtgcaggggatccttctcatctcctggaccctccagggcactctggtccctattccccagctcctaggcagctgagccgggtcccttaggggaggtgaccaggagctttggtgcagggagctcttggtggggcaaagggctggacccctgccaggtctgtggacatggttatatgcccgggagaggggggtgcagggccccagggatggcccccaatcccacctctgtttattctgtaaactgcaacctataaataacctttagcattcctattgtaacaaaattaatttttatgaaataaattatatttcctagtctaataaaaaaaaaaaaaaaaaaaaa

SEQ ID NO: 154 - DNA, 1130 bp, Homo sapiens
tatttttttttttactgatatattgtagtttaataaaacatagtttatacagttcattgaaaaagtattttaatacaaacaccacttatacacaaaaccaaatgttgatattcttgtttttaaaaattcttgatttctctaaaacactaagatgctatctcaatagagattgcttcacattttccagtttcttgatctgtgcatgtcacatgtaaagatccatcccttttcatagtaagaacagctaatatatcacgtaatccattttcttttttatctaaatcctggagtacaacctgtgcaaacttggtttcctctttggcagagttcttcccatcagactcatagagttcaaggcacactgaagatatgcttccaggggcttgcaatgtgtgttgtcttcgagctggcaaaggagtccctgatggaaacagcactgtgaatctactggctcctgattcgtccacacccttaactaaaatatctctggctgaacactctatcataagagagtcttccaccaacaggttttctttcccaataagaattcctgcttctatagctgcaccaatagggatcacttcatcaggagggatagaattgagaagctcaacagctgggaaaagatctttaatcagttgctgtagctttgggattcgagaagaccctccacaaaggacaaccttgttgatatcatctgctgtaaatccattttgatctaagagtcctctgattgcttctatacacttattaaaaagtggagaacaaagaagttcaaatcttgctctggacacattgcaatcaaaatcttgaccttcatataatgagtcaagaaaacagttggcacttcccaaggttgacaaagaatgtttcgctacttcagcactgttcgttaatttcatcatggctcgcgcatttcctctcacatcatgtttgaaggatctttggaactcagaagctagatactgtgctaaggtttctgtgaaatgtgcaccaccgatgttatcatcagtgtttgttgaaagaacccgatatattccactgttaacttccatgacgctgagagataaggatgttcctccaagcttaaacaccaaaatattgctttttccagtaggggagtcttgtcc

SEQ ID NO: 155 - DNA, 5779 bp, Homo sapiens
ttgcgggaaagagccaaaccctggcgttggggggcccgggcggggagcccctcccgcggtccacagcgacgcctgcccagccctcctccccttccggctccggcacggggccccgaggcgttcggaggccaggcgggtttctgtcaggcccggggaggaggggcgggcggggcggccgctgcctccccgggacgggccgtaccacgcggacggggaggacggggccaggggactgcagggcggctgcaccgcccgggggcggggtgcggacgggccggcgggctccccggggcggggcgggagggcggggcgtggggcggacggaaccaccggggcggggtgggaggtaacgggacgggcgcgaccatggcgcggtgagggagcgggggtggggatcggtccgggggaggcctgaggccgctggcttgtgcgctgtctccgccgcccccctctttcgccgccgccgccgccgccccgggcatgtcgtccaactgcaccagcaccacggcggtggcggtggcgccgctcagcgccagcaagaccaagaccaagaagaagcatttcgtgtgccagaaagtgaagctattccgggccagcgagccgatcctcagcgtcctgatgtggggggtgaaccacacgatcaatgagctgagcaatgttcctgttcctgtcatgctaatgccagatgacttcaaagcctacagcaagatcaaggtggacaatcatctcttcaataaggagaacctgcccagccgctttaagtttaaggagtattgccccatggtgttccgaaaccttcgggagaggtttggaattgatgatcaggattaccagaattcagtgacgcgcagcgcccccatcaacagtgacagccagggtcggtgtggcacgcgtttcctcaccacctacgaccggcgctttgtcatcaagactgtgtccagcgaggacgtggcggagatgcacaacatcttaaagaaataccaccagtttatagtggagtgtcatggcaacacgcttttgccacagttcctgggcatgtaccgcctgaccgtggatggtgtggaaacctacatggtggttaccaggaacgtgttcagccatcggctcactgtgcatcgcaagtatgacctcaagggttctacggttgccagagaagcgagcgacaaggagaaggccaaggacttgccaacattcaaagacaatgacttcctcaatgaagggcagaagctgcatgtgggagaggagagtaaaaagaacttcctggagaaactgaagcgggacgttgagttcttggcacagctgaagatcatggactacagcctgctggtgggcatccacgacgtggaccgggcagagcaggaggagatggaggtggaggagcgggcagaggacgaggagtgtgagaatgatggggtgggtggcaacctactctgctcctatggcacacctccggacagccctggcaacctcctcagctttcctcggttctttggtcctggggaattcgacccctctgttgacgtctatgccatgaaaagccatgaaagttcccccaagaaggaggtgtatttcatggccatcattgatatcctcacgccatacgatacaaagaagaaagctgcacatgctgccaaaacggtgaaacacggggcaggggccgagatctcgactgtgaaccctgagcagtactccaaacgcttcaacgagtttatgtccaacatcctgacgtagttctcttctaccttcagccgagaccgagagactggatatggggtcggggatcgggacttagggagaagggtgtatttgggctagatgggagggtgggagcgagatcgggtttgggagggctttagcaatgagacttgcagcctgtgacaccgaaagagactttagctgaagaggagggggatgtgctgtgtgtgcaccagctcacaggatgtaaccccaccttctgcttacccttgattttttctccccatttgacacccaggttaaaaaggggttccctttttggtaccttgtaaccttttaagataccttggggctagagatgacttcgtgggtttatttgggttttgtttctgaaatttcattgctccaggtttgctatttataatcatatttcatcagcctacccaccctccccatctttgctgagctctcagttcccttcaattaaagagatacccagtagacccagcacaagggtccttccagaaccaagtgctatggatgccagattggagaggtcagacacctcgccctgctgcatttgctcttgtctggattaactttgtaatttatggagtattgtgcacaacttcctccacctttcccttggattcaagtgaaaactgttgcattattcctccatcctgtctggaatacaccaggtcaacaccagagatctcagatcagaatcagagatctcagaggggaataagttcatcctcatgggatggtgaggggcaggaaagcggctgggctcttggacaccctggttctcagagaaccctgtgatgatcacccaagccccaggctgtcttagcccctggagttcagaagtcctctctgtaaagcctgcctcccactaggtcaagaggaactagagtacctttggatttatcaggaccctcatgtttaaatggttatttccctttgggaaaacttcagaaactgatgtatcaaatgaggccctgtgccctcgatctatttccttcttccttctgacctcctcccaggcactcttacttctagccgaactcttagctctgggcagatctccaagcgcctggagtgctttttagcagagacacctcgttaagctccgggatgaccttgtaggagatctgtctcccctgtgcctggagagttacagccagaaaggtgcccccatcttagagtgtggtgtccaaacgtgaggtggcttcctagttacatgaggatgtgatccaggaaatccagtttggaggcttgatgtgggttttgacctggcctcagccttggggctgtgttttccttgttgccccgctctagacttttagcagatctgcagcccacagggtcttttttggaaggagtggcttcctccaggtgttccacctgcttcggagcctgccacccaggccctcagaactgagccacaggctgctctggccaggagagaaacagctctgttgttctgcattgggggaggtacattcctgcatcttctcaccccctcaaccaggaactggggatttgggatgagatatggtcagacttgtagataaccccaaagatgtgaagatcgcttgtgaaaccattttgaatgaatagattggtttcctgtggctccctccaaacctggccaagcccagcttccgaagcaggaaccagcactgtctctgtgcctgactcacagcatataggtcaggaaagaatggagacggcattcttggacttcactggggctgctggattggatgggaaaccttctggaagaggcagatgggggtcaaaccactgcccttgccccaggaaggggccataggtaggtctgaacaactgccgcaagaccactacatgacttagggaacttgaaaccaactggctcatggagaaaacaaatttgacttgggaaagggattatgtaggaataatgtttggacttgatttccccacgtcataatgaagaatggaagtttggatctgctcctcgtcaggcgcagcatctctgaagcttggaaagctgtcttccagcagcctccgtggcctcgggttcctaccggcttctctgcatttggtctgctgatcatgttgccataatgtgtatggaaagtgtaacacattcttactggttaaagacgactaccaggtatctaacttgtttaacattgagattgtgtgtgtgtgtgtatgtttgtgtgttttgtatattgtttacattttgagaggtagcattctgtttcaaatgctttttgtttttctgaccagtattgttgactgggtcataacattttgagctgtggtttggtggattttcaatttttttttttaaaggtcattcgctgtgctatcttcaaaaccttgagtttggcccccaatttttggcattcaaatgtttaaaagctatttatcttggtttatacaagtttcctttctcttctttttgtcatggtattctatttggtctgcagtttgaatgtagagaaagtggactgatcccccaagcgttgtctgcccccactctttcctccttgggtcccgccattcttttactgggcagtcgagggcattggaggggaagtgactgccctcagcctcactccctggggccatgaagaaaagctaaacagtctcatggcatctcagaataatgttgggtctcccaagaagaaaggtgtaagaataacgacatggctgattaggcgaggccaggatagggctaaggccaggattcctggctggcatccagtcaccccttctcccatccttccccctcttcttccacaagtccgcagccgagacactgtagtctcccagccacagtgatgagtgccctggagactccactgacctctagatgaaggcccctggccctggttcctgttaattaacctctgggtctttgagtcccccagcacaaacttctttcctgtaccctgcggcttggggtcacagggcatgccgggaagccacagctgaggggcgcagactgaagcagtgctccacctctccttctttagctcaggggttgctggtctgtggcaggcgccacgagtggcccctgtggctgttctcagtggcagtctcttaagttcccaccacaggcagctctttatcccctctccctacttgactctttctcttgcctgtgcttttggcctcaaacaggcctgctggtagcgctcagggcgtgaggctacactcctgccctgcctttcctgtcttcatggtctgccagggcataccttggggaggtggaccaaagacccaggactttttgcagtagccagtcctaccccccagttgtctttttaccaattcagggtgggagagaaaactgcagcaccccagcatgtgagttactcaggtgttgggggctagaagggacagtgcgtttaaacaacactcagagctctggccttaaacctgtggccccccaagtctaggagcctcatctcttcctggcagtcatgcgggcaggaggtcctgaaagggaaaacccattcagacaactgttccccaatctaccagccatctgcaggggtcagtgaccgtggccctctccctcctctagaatgtgccacttatgaagagtgccccatggggaaaaggagactcagctgtcccttggcagcttgtgccagtatcccagggcagaagtttccacaggagcctcttgcccttgcgcagagccactgtgagaggcggtgggagccaacacccttgggggagggggcagtactgctcggcacatcccagcatcaggtcagatcactgaaattaaaaaatgtgaattaagttcatatccaccttttggggaagcaggacaaaccaccaccccaccaagtgtgtgacttctccatatcccactgcagtttccattttttaaatgggaattttcaatcccctgtgcttgtctaacgtctgctttaaaaagtttgagaccctgttactgtttgaaaatgcatgcatgttacgatgaatctccaacctgaggaaaaaaataaaactcaaaaagctttgtgtaaaaaaaaaaaaaaaaaagaatgagaggagagcagggggcggg

SEQ ID NO: 156 - DNA, 2408 bp, Homo sapiens
gcacgcccttcggccgcggccgggccgcccgctccttccttccgcttgcgctgtgagctgaggcggtgtatgtgcggcaataacatgtcaaccccgctgcccgccatcgtgcccgccgcccggaaggccaccgctgcggtgattttcctgcatggattgggagatactgggcctgttaggcctgttacattaaatatgaacgtggctatgccttcatggtttgatattattgggctttcaccagattcacaggaggatgaatctgggattaaacaggcagcagaaaatataaaagctttgattgatcaagaagtgaagaatggcattccttctaacagaattattttgggagggttttctcagggaggagctttatctttatatactgcccttaccacacagcagaaactggcaggtgtcactgcactcagttgctggcttccacttcgggcttcctttccacagggtcctatcggtggtgctaatagagatatttctattctccagtgccacggggattgtgaccctttggttcccctgatgtttggttctcttacggtggaaaaactaaaaacattggtgaatccagccaatgtgacctttaaaacctatgaaggtatgatgcacagttcgtgtcaacaggaaatgatggatgtcaagcaattcattgataaactcctacctccaattgattgacgtcactaagaggccttgtgtagaagtacaccagcatcattgtagtagagtgtaaaccttttcccatgcccagtcttcaaatttctaatgttttgcagtgttaaaatgttttgcaaatacatgccaataacacagatcaaataatatctcctcatgagaaatttatgatcttttaagtttctatacatgtattcttataagacgacccaggatctactatattagaatagatgaagcaggtagcttcttttttctcaaatgtaattcagcaaaataatacagtactgccaccagattttttattacatcatttgaaaattagcagtatgcttaatgaaaatttgttcaggtataaatgagcagttaagatataaacaatttatgcatgctgtgacttagtctatggatttattccaaaattgcttagtcaccatgcagtgtctgtatttttatatatgtgttcatatatacataatgattataatacataataagaatgaggtggtattacattattcctaataatagggataatgctgtttattgtcaagaaaaagtaaaatcgttctcttcaattaatggcccttttattttgggaccaggcttttattttccctgatattatttctatttaatactcttttctctcaagaaaaaaaaaaaagtttgttttttctttattgtccttcatagcaggccaagtattgcctctctgcaatagacagctactgtcaatacatgctgtaatttgacattctgggtcacagatataaggtatttaaaatctatttatgctttatagagaaaccagacattaaaacttcatgcactacttatttcgaattactgtaccttatccaaatttacacctagctattaggatcttcaacccaggtaacaggaataattctgtggtttcatttttctgtaaacaactgaaagaataattagatcatattctagtatgttctgaaatatctttaagactgatcttaaaaactaacttctaagatgatttcatcttctcatagtatagagtttactttgtacacgtttgaaaccaactactgtagaagatgaggaatctattgtaattttttgctttattttcatctgccagtggacttatttgaaattttcactttagtcaaattattttttgtattagtttttgatgcagacataaaaatagcaatcattttaaattgtcaaaatttccagattactggtaaaaattatttgaaaacaaacttatgggtaataaaggctagtcagaaccctataccataaagtgtagttaccatacagattaatatgtagcaaaaatgtatgcttgatatttctcaactgtgttaatttttctgctgtattccagctgaccaaaacaatattaagaatgcatctttataaatgggtgctaattgataatggaaataatttagtaatggactatacaggatgttaataatgaagccatatgtttatgtctggatttaaaaattttaaacaatcatttactatgtcatttttctttaccttgaagaacataaactgttatttcacttctacaaatcagcaagatattatttatggcaagaaatattccattgaaatattgtgctgtaacatgggaaagtgtaaatgtttttcatggtttctatcaatgtgaaataaaatttaattctgaaaaaaaaaaaaaaaaaaaaagagag

SEQ ID NO: 157 - DNA, 1548 bp, Homo sapiens
ctgggctttcacgctgctgtgcgaggaactgcgccagagcctgagcggaggctgggggcagcctcgccagcgggggccccgggcctggccatgcctcactgagccagcgcctgcgcctctacctcgccgacagctggaaccagtgcgacctagtggctctcacctgcttcctcctgggcgtgggctgccggctgaccccgggtttgtaccacctgggccgcactgtcctctgcatcgacttcatggttttcacggtgcggctgcttcacatcttcacggtcaacaaacagctggggcccaagatcgtcatcgtgagcaagatgatgaaggacgtgttcttcttcctcttcttcctcggcgtgtggctggtagcctatggcgtggccacggaggggctcctgaggccacgggacagtgacttcccaagtatcctgcgccgcgtcttctaccgtccctacctgcagatcttcgggcagattccccaggaggacatggacgtggccctcatggagcacagcaactgctcgtcggagcccggcttctgggcacaccctcctggggcccaggcgggcacctgcgtctcccagtatgccaactggctggtggtgctgctcctcgtcatcttcctgctcgtggccaacatcctgctggtcaacttgctcattgccatgttcagttacacattcggcaaagtacagggcaacagcgatctctactggaaggcgcaggttaccgcctcatccgggaattccactctcggcccgcgctggccccgccctttatcgtcatctcccacttgcgcctcctgctcaggcaattgtgcaggcgaccccggagcccccagccgtcctccccggccctcgagcatttccgggtttacctttctaaggaagccgagcggaagctgctaacgtgggaatcggtgcataaggagaactttctgctggcacgcgctagggacaagcgggagagcgactccgagcgtctgaagcgcacgtcccagaaggtggacttggcactgaaacagctgggacacatccgcgagtacgaacagcgcctgaaagtgctggagcgggaggtccagcagtgtagccgcgtcctggggtgggtggccgaggccctgagccgctctgccttgctgcccccaggtgggccgccaccccctgacctgcctgggtccaaagactgagccctgctggcggacttcaaggagaagcccccacaggggattttgctcctagagtaaggctcatctgggcctcggcccccgcacctggtggccttgtccttgaggtgagccccatgtccatctgggccactgtcaggaccacctttgggagtgtcatccttacaaaccacagcatgcccggctcctcccagaaccagtcccagcctgggaggatcaaggcctggatcccgggccgttatccatctggaggctgcagggtccttggggtaacagggaccacagacccctcaccactcacagattcctcacactggggaaataaagccatttcagaggaaaaaaaaaaaaaaaaaaaaaaa

SEQ ID NO: 158 - DNA, 2319 bp, Homo sapiens
  modified_base (165): a, t, c, g, other or unknown
aacgtcattggtaacagcaagtcccagacaccagcccccagttccgaagtggttttggattcaaagagacaagttgagagagaggaaaccaaccatgagatccaggaggggaaagaagagcctcagagggacaggctgccgcaggagccaggccgggagcaggtngtggaagacagacctgtaggtggaagaggcttcgggggagccggagaactgggccagaccccacaggtgcaggctgccctgtcagtgagnccaggaaaatccagagatggagggccctgagcgagaccagcttgtcatccccgacggacaggaggaggagcaggaagctgccggggaagggagaaaccagcagaaactgagaggagaagatgactacaacatggatgaaaatgaagcagaatctgagacagacaagcaagcagccctggcagggaatgacagaaacatagatgtttttaatgttgaagatcagaaaagagacaccataaatttacttgatcagcgtgaaaagcggaatcatacactctgaattgaactggaatcacatatttcacaacagggccgaagagatgactataaaatgttcatgagggactgaatactgaaaactgtgaaatgtactaaataaaatgtacatctgaangatgattattgtgnaaattttagtatgcactttgtgtaggaaaaaatggnaatnggtcttttaaacagcttttggggggntactttnggaagtgtctnaataanggtgtcacnaatttttggntagtanggtatttcgtgagnaagnnttcaacaccaaaactnggaacatagttctccttcaagtgttggcgacancgggnngcttcctgattctggaatataactttgtgtaaattaacagccacctatagaagagtccatctgctgtgaaggagagacagagaactctgggttccgtcgtcctgtccacgtgctgtaccaagtgctggtgccagcctgttacctgttctcactgaaaagtctggctaatgctcttgtgtagtcacttctgattctgacaatcaatcaatcaatggncctagangcactgactgttaacacaaacgtcactagncaaagtagncaacnagctttaagtctaaatacaaagctgttctgtgtgagaattttttaaaaggctacttgtataataacccttgtcatttttaatgtacaaaacgctattaagtggcttagaatttgaacatttgtggntctttatttactttgcttncgtgtgtgggcaaagcaacatcttccctaaatatatattaccaagaaaangcaagaagcagattaggntttttgacnnaaaacanaacaggccnnaaaagggggcntgnacctggagcagagcatggtgnagaggcaaggcatgnagagggcaagtttgttgtggacagatctgtgcctactttattactggagtaaaangaaaacaaagttncattgatgtcgnaaggatatatacagtgttnagaaattnnaggnactngtttnagaaaaacaggaatacnnaatggnttgntttttatcatangtgntacacatttagcttgtggntaaatngactcacaaaactganttttaaaatcaagttaatgtgaattttgaaaattactacttaatcctaattcacaataacaatggcattaaggtttgacttgagttggttcttagtattatttatggtaaataggctcttaccacttgcnaaataactggnccacatcattaatgactgacttcccnagtaanggctctctaaggggtaagtnaggaggatccacaggatttgagatgctaaggccccagagatcgtttgatnccaaccctcttattttcnagaggggaaaatggggcctnagnaagttacangagcatcntnagcntggtgcgctggncaccccntggccntcnacacnagactcccngagtagctggganctacaggcacacagtcactgaagcaggcccntgtttgcaattcacgttgccnacctnccaacnttaaacattncttcatatgtgatgtccttagtcacntaaggttaaanctttncccacccagaaaaggcaacttagataaaatcttagagtactttcatactcttctaangtcctcttccagcctcactttgagtcctccttnggggttgatnnnaggaattttctcttgctttctcaataaagtctctattcatctcatgtttaatttgtacgcatagaattgctgagaaataaaatgttctgttcaacttannnnnaaaaaaaaaanaaaaaaaaaaa

SEQ ID NO: 159 - DNA, 1467 bp, Homo sapiens
cgaggaccggccttgcgagcggcgacgactataaaatggcgcgtgctgcaacccgcgcccgcttcggagagagaaatgctggggtgcagcttcaagcttaggaccacccaccatgcctatccaggtgctgaagggcctgaccatcactcattaagaacagaggaggctgcctgttactcctggtgttgcatccctccagacactctgctgtttcctgcctaggcgtggctgcagccatggctaggaaagcgctgccacccacccacctgggccagagctggttctgctcctgctgcagggacactgagctggctatctcggcgcttcgggcaagaactgcaacaggctctcctgggtcctgcaggtgtacagccgggcccctgccttgtgcctcagctctcgagagctgctgctgccgggtgacctgatccaacctgataaggtgccatcttcagctaccactgcaaggccctgagggcaacagcagcacggcactgcccacccggctgctgatggcctggtgccagctgggagtcctcccggcacttcgaggccactgagccacccttccagccccagcccaccatggacaggggtatccagcttcctcctcaacctcgtcctctgcccctgagccagtgacgcccaaggacatgcctgttacccaggtcctgtaccagcactagctggtcaagggcatgacagtgctggaggccgtcttggagatccaggccatcactggcagcaggctgctctccatggtgccagggcccgccaggccaccaggctcatgctgggacccaacccagtgcacaaggacttggctgctgagccacacacccaggagaaggtggataagtgggctaccaagggcttcctgcaggctaggggaggagccacccccgcttccctattgtgaccaggcctatggggaggagctgtccatacgccaccgtgagacctgggcctggctctcaaggacagacaccgcctggcctggtgctccaggggtgaagcaggccagaatcctgggggagctgctcctggtttgagctgcattcaggaagtgcgggacatggtaggggaggcaaaaagccttgggcactaccctccctgtggagctgttcggtgtccgtcgagctagccacaccctgacaccatgttcaagggtaccggaagagaagggtgtctgcccccaacctcccctgtgggtgtcactggccagatgtcatgagggaagcaggccttgtgagtggacactgaccatgagtccctggggggagtgatcccccaggcatcgtgtgccatgttgcacttctgcccaggcagcagggtgggtgggtaccatgggtgcccacccctccaccacatggggccccaaagcactgcaggccaagcagggcaaccccacacccttgacataaaagcatcttgaagcttttaaaaaaaaaaaaaaaaaaaaaataa

SEQ ID NO: 160 - DNA, 1348 bp, Homo sapiens
cgtaactgcaggttgtgtgagtgcgttaaaaaaaaaaaaaaaaaaaagaatccctatacctcatttgtatttttaaaatgcgtgatgttttatgaaattgtgtccattttttaggtattagatatggcagaaaaaccatttccactatgcaaagttcttttagacgtcagtgaaaatcaactctcatacctcatggtctctctttaattgaccaaaaccttccatttttctctaaatacaaagcgatctgtgttctgagcaacctttccccgaacacacagcttcagtgcagcacgctgacctgagtatccaccatgtgccaggcacagtgctgggcacacgaggcaccaaggtccgggccacctgcccgcagcaaggcccagctgaggtggtggagggagcccctgaggtcaggggccgtttcggttcagggtggcaggtgtccagcactggggtatggcgtcgaggcttccatggggtgggggaggccagcttccttctgacaggatgggcgcatacagtgcctggtgtgatttgtgcacaacccgtgttccaggtgcacatcctcccaaggagacacccagacccttccagcacgggccggccaagttgctgcggcggaggcagcatttcagctgtgaggaaggtcattggattcatgtgttttatctgtaaaaatggttgtcttaacttcttaacctcatattggtaagtgattgataaaaattggttggtgtttcatgacatgtggacttcttttgaaatagcaagtcaaatgtagtgaccaaattgtggaagagatttctgtcaaataggaaatgtgtaagttcgtctaaaagctgatggttatgtaagttgctcaggcactcagatgacagcagattctgggttctgggagtgttctgtgcctcttacatgccctggaggcctcatggtctcagtgctgaggcggcacacctgtagcacacctgcgtaatgtgcggtctgggccagtcacaaggaattgtgttgtctaagccaaagggggaagctgactgtgatttaccaaaaaaaattctgtaattcaaaccaaaatgtctgcggaatcaccagtttgatactctctgtaatcagaacagtgggcagtgcctgggtgaacgtgtctagcagccactgtgcgggatcgctgtaacaggagtggaatgtacatatttatttacttttctaactgctccaacagccaaatgccttttttatgaccattgtattcagttcattaccaaagaaatgtttgcactttgtaatgatgcctttcagttcaaataaatgggtcacattttcaaatggaaaaaaaaaaaaaaaaaaaaaa

SEQ ID NO: 161 - DNA, 1290 bp, Homo sapiens
cccaactccacccagggatcctggtgtacgggctgacctgttatgcttttctgcccttcggccctttggggagccacggcgggaggtggagatccaccggcgatatgtggcccagtcggtccagctctttattctctacttcttcaacctggccgtgctttccacttacctgccccaggataccctcaaactgctccctctgctcactggtctctttgccgtctcccggctgatctactggctgacctttgccgtgggccgctccttccgaggcttcggctacggcctgacgtttctgccactgctgtcgatgctgatgtggaacctctactacatgttcgtggtggagccggagcgcatgctcactgccaccgagagccgcctggactacccggaccacgcccgctcggcctccgactacaggccccgcccctggggctgagcctctccgccctcgccctcggagtagggggtagcggcttgggtctgacacatctttgaaccttgtggccaggcctggacttcgcccccaggcctaggaccgcggtgggtggaaccctgctactgccccaacagggactccaatcaatcggagttctccccttgccggagctgcccttcacctttggggcccgagacagtcataagggatggacttagttttcttgcagggaaaaaggtggacagccgtgtttcttaaggatgctgagggcatggggccaggaccaggggagaggcacagctccttcctgagcagcctctcaccactgccacaaggctccctaatgctggtctctgctccactccccggcttcccgtgaggcaggaggcagagccacagccaaggccctgaccacttctgtgccagttgtctaagcagagcgcctcagggacgctggaaatgccttaaggatagaggctgggcatcacatcaaatgggactgtggtgtttggtgaaaaccttcctgaggatctggattcaggaccctccatgactggcctatttactgtttacagctggccagtgcagagctgctgctcttttacctttttaggcccctgtaacttcccacctttaaactgcccagaaggcatgcctctcccacaggaagaggggagcagacagggaaatctgcctaccaagaggggtgtgtgtgtctttgtgcccacacgtggtggctggggagtgcctggatggtgcggtggttgatgttaacctagtgtgtgtgtgtgtgtgtgtgtgtgtgtgtgtgtgtgtgtgtgtaacaataaattactaccagtcaaaaa

SEQ ID NO: 162 - DNA, 2912 bp, Homo sapiens
  modified_base (915): a, t, c, g, other or unknown
gtcgaccgtggccgctggctggctggacaggcgggtgtgaggagttgcagacccaaacccacgtgcattttgggacaattgctttttaaaacgtttttatgccaaaaatccttcattgtgattttcagaaccacgtcagatataccaagtgactgtctgtggggtttgacaactgtggaaaggcgagcagaaaactccggcggtctgaggccatggaggtggttgctgcatttgagagggagtagggggctagatgtggctcctagtgcaaaccggaaaccatggcaccttccagagccgtggtctcaaggagtcagagcagggctggccctcagtagctgcagggagctttgatgcaacttatttgtaagaaggatttttaaattttttatgggtagaattgtagtcaggaaaacagaaagggcttgaaatttaataagtgctgctggaaggggatattccaagccggcaagaggattcagcagctgtggtggggaaacatttctcctgaaagactgaacgtgtttcttcatgacagctgctcaaagcaggtttctgagatagctgaccgagctatggtaaatctctttgtcaaattacgaaaacttcagggtgaaatcctatgcttccatgtacattacatggcttaagattaaacaaaaacatttttcaagtctctaactagagtgaactctagagcacagtagttcagaaactatttagagcttccaggatatatttcacagcttcaggcatgtgatcagttagagccgatgaaacctatgcccgcctgtatatatattagcagcttagctagttcataacctgtatattctaaagactgctaaggttttgttttcattttaaatcctagctgattgttgtggtcaatgaaatacccagtttctggagggnccaggtgggaaatgctttcactggaccaacacacaaatgatcatcctgnaggatctgagcttccctangactccacacaataacngttggggcacccnttttangagaagactgttgaaacccacagcactcgttggggtatgaggaaaccagggncttggcacaggaagttcccctttgtagctaaaagtccagaaagaaagggttcatctttttgacttccaactgatattgggaagtttggttgaggttcaagtgtgactccttccagagccacaggtaggggagtgtgaagttgagggggaggaaagctggaaggactctgccttgggagattcccagctctgctttccagcgcttggtggnaatctgggctggggaaagacnggcaccngggaaactctgcttccccattgtttccatctgatcagctgtggtgtgaggacttctcagacaaaggcaaggcctcngtgcccctgcccagcccattcatggagccctgggccttcttggcttccatagatcctaagctcttgactgtagtttagccagacttgttttgctatcttatnaagcagttcagaattangggaatgctggttttgaagagcaaaggacaggtagtctagnagagggntcgtctggncctgcttgctgggntcnntttgtaacccagcacttccntcttgccctcctggctttatgtttatnggggagaggactcaatnagctccaccccttcttggcaccagatggggctttggttagtttngcaataagcaccttgcaganggnttaaagccagcgggtcccntagtcttnnaggcccagcctgctgtgtgtgggctctggcctggcctggtggctggcccagggcggagcaagtgcttagagcttctgcagggcttctcttgtttacacagctgcatcagacaatgccatttctccccaccacggaaccttccatctaagatttcttccangggaatgccagcaatcaggcagcacccagctgtgggggcagtggggtgggggagacccacattgatgactttttttttttcttttaatgaagaaacaccaaagaaagctgtggaaaggacctgccccacatgaaaaggataagccaagatggctgtaaacacagagcatttgagctgccactcttggagcacattgatttttcaaaagccagctctgtcaggaaaggaggtgctgttatgagcagctcttccagtgggncaaagaggacgcccatnaatttcttccattgctagctcatctgtggggaccaatttggtgttaagcaacctgtggcctgcacttgtggcctcgaaggaagcacaaaccctccatccacttcccatttcctctgcccttttccacctcccccttccatcccaccagctgccnagtggctncccagaaagccttattgagccccttgttgacacttggggctgcggnaggcctctcccntactggtctggcctttcctgagaggcaggtcttccgtcctcagagcctttctggaacaaggagaatgcctgtgnncaggtggnnacacnacnaggcctggcctgtncgctnctcnacttgtcttnccagcggggangcttncacgttgccngagtggaangaanccatgaccnntccacttngcttnccaaggntgnctagggaagtttcagggtacgctggnttcccctcntccagctgganggccgagtttctggggnacntgcagatttttnctactctgtgantcgattcaatgcccgatgcttctgtttncattcccgaccctttctactatgcattttccttttatcaggtgtataaagttaaatactgtgtatttatcactaaaaagtacatgaacttaagagacaactaagcctttcgtgtttttccacaggtgtttaagcttctctgtacagttgaaataaacagacagcaaaatggtgccaanaaaaaaaaaaaaaaaaaaaaaaaaa

SEQ ID NO: 163 - DNA, 850 bp, Homo sapiens
ccgtgttgtacggcggacttctcgcgcagtgaatgacctggaagtgatgcctaaagctgtggaccgcgcgggatcgcctccctgggactaggtttcagcggccgctgcgatgaccaaaataaaggcagatcccgacgggcccgaggctcaggcggaggcgtgttccggggagcgcacctaccaggagctgctggtcaaccagaaccccatcgcgcagccctggcttctcgccgcctcacgcggaagctctacaaatgcatcaagaaagcggtgaagcagaagcagattcggcgcggggtgaaagaggttcagaaatttgtcaacaaaggagaaaaagggatcatggttttggcaggagacacactgcccattgaggtatactgccatctcccagtcatgtgtgaggaccgaaatttgccctatgtctatatcccctctaagacggacctgggtgcagccgcaggtccaagcgccccacctgtgtgataatggtcaagccccatgaggagtaccaggaggcttacgatgagtgcctggaggaggtgcagtccctgcccctacccctatgaggggctccggtagcacctgggcacctgccgctggaagctattgggctggcagcaggacgactggctgtcctcctgcccacccacactgacggcatcttcccagttccccaaggcacgccttcttcccaggcagctctaacagccctttcatgaaggtaatgctagtcttctgtccatcagtgccatttcctgtagaactaaaggctgttccaagaatgtggggtggggaaagtaaatgctaagactaaaaaaaaaaaaaaaaaaaaaaaaaaaaaaaaaaa

SEQ ID NO: 164 - DNA, 2223 bp, Homo sapiens
ggtgggtggagccaggcttggcgggctgtgcgtgctcgcggtgggcggtggcggcggctgcctcgcgaaggttcgagatccgtcgcgtgcgggaggcgggccgcgatcttgcgcagggtcggtgtgggcgcaggctgcagcgccgcgactcgtgcgggtaggcgtctgcgctcggtttgagggctcggcgcggggtttcctgttccttcttctgcgcggctgcagctcgggacttcggcctgacccagcccccatggcttcagaagagctacagaaagatctagaagaggtaaaggtgttgctggaaaaggctactaggaaaagagtacgtgatgcccttacagctgaaaaatccaagattgagacagaaatcaagaacaagatgcaacagaaatcacagaagaaagcagaacttcttgataatgaaaaaccagctgctgtggttgctcccattacaacgggctatacggtgaaaatcagtaattatggatgggatcagtcagataagtttgtgaaaatctacattaccttaactggagttcatcaagttcccactgagaatgtgcaggtgcatttcacagagaggtcatttgatcttttggtaaagaatctaaatgggaagagttactccatgattgtgaacaatctcttgaaacccatctctgtggaaggcagttcaaaaaaagtcaagactgatacagttcttatattgtgtagaaagaaagtggaaaacacaaggtgggattacctgacccaggttgaaaaggagtgcaaagaaaaagagaagccctcctatgacactgaaacagatcctagtgagggattgatgaatgttctaaagaaaatttatgaagatggagacgatgatatgaagcgaaccattaataaagcctgggtggaatcaagagagaagcaagccaaaggagacacggaattttgagactttaaagtcgttttgggaactgtgatgtgatgtggaaatactgatgtttccagtaagggaatattggtgagctgcatatataaatttgacagatagctatttacatagccttctaagtaaaggcaatgaattctccatttcctactggaggatttatttaaataaaatatgcttattaaacactcctgcaaagatggttttattagtaccctggtcattttgttcaaggaagggttatattgcattctcacgtgaaatataaaaagcaagtcttgcccaataaaaacgctacattgtgtgtattttttgttcagctaagaattggaaaagtatttgcttgccttttaagttactgacatcagcttccaccagtgtaaaaattgagtaaaacctgaagttttgcataaaatgcaaatcggtgcctgtgcttgaaggttgctgtagagcatctgaccccttattaccaccttaagcaatgtatatgccatgcattaccatgcactaattcaatcacaggtgtttctatctagatttaaatatatttgtcaatgaatgtggaatagaaaatctaaacatgacaataatagacatatctttgtatggtaccagttagttttgccgtggatcagatggtttataaaagtaataaccataaagcaaaaaataatttgaaagcccgtctattcctatgctcaataaagttaagtttttcttcattagaacagttttatgatttatttgtctaggagtatgtcagaaaaatcaggcttttagtaggaattactcctattccccctgaagtcaggaccagtgcctgtgatctccattactttattttcctggaggtattagccaacacagttagatcagagaaagcaattgaagccaggcatgaagtggcgcccgtaatcccagctactaaggctggaggatcacttcagcccaggagtttaaggctgcagtgagctatgatgatgccactgtactgcagcttgggtaacagattgagaacctgtctcattaaaaaaaaaaaaaaaaaaaaagccgttagacacacaggaaaaatccagaagggtaaactaaactaaagtccaattaatatgggaatttggaagaagtggtaggatttaaaatacagaaccagtttatgttaggatagtataagtaaatctgaacacattatggcctctgtaattggggttgcacatgacagatggcagcacaatcattgaaagttcagatatggtaaagtgggtagatggttttatgtctagtactga

SEQ ID NO: 165 - protein, 546 aa, Homo sapiens
QDFIGGEPTPGRYCHGGFGVLLSRMLLQQLRPHLEGCRNDIVSARPDEWLGRCILDATGVGCTGDHEGVHYSHLELSPGEPVQEGDPHFRSALTAHPVRDPVHMYQLHKAFARAELERTYQEIQELQWEIQNTSHLAVDGDRAAAWPVGIPAPSRPASRFEVLRWDYFTEQHAFSCADGSPRCPLRGADRADVADVLGTALEELNRRYHPALRLQKQQLVNGYRRFDPARGMEYTLDLQLEALTPQGGRRPLTRRVQLLRPLSRVEILPVPYVTEASRLTVLLPLAAAERDLAPGFLEAFATAALEPGDAAAALTLLLLYEPRQAQRVAHADVFAPVKAHVAELERRFPGARVPWLSVQTAAPSPLRLMDLLSKKHPLDTLFLLAGPDTVLTPDFLNRCRMHAISGWQAFFPMHFQAFHPAVAPPQGPGPPELGRDTGRFDRQAASEACFYNSDYVAARGRLAAASEQEEELLESLDVYELFLHFSSLHVLRAVEPALLQRYRAQTCSARLSEDLYHRCLQSVLEGLGSRTQLAMLLFEQEQGNST

SEQ ID NO: 166 - protein, 267 aa, Homo sapiens
TAAACMPSPAGRPSFPCISKPSTQLWPHHKGLGPQSWAVTLAALIARQPARPASTTPTTWQPVGAWRQPQNKKRSCWRAWMCTSCSSTSPVCMCCGRWSRRCCSATGPRRAARGSVRTCTTAASRACLRASAPEPSWPCYSLNRSRATAPDPTLSPWAVAWPHPTPLLPQNQSHLPASLGRAGRSQTPSWPTGPLSGSVGPWALDKHWGTCPQSHPLLIPNPVSLPPDAADSGCGLHVFMQYSLPDASPASGPWGLGCRRVVGEGGS

SEQ ID NO: 167 - protein, 138 aa, Homo sapiens
MKRQLGRLLLLRRVLDPLLCDFLDSQDSGSLCFCFRWLLIWFKREFPFPDVLRLWEVLWTGLPGPNLHLLVACAILDMERDTLMLSGFGSNEILKHINELTMKLSVEDVLTRAEALHRQLTACAEGPTTCRRSGVGRP

SEQ ID NO: 168 - protein, 250 aa, Homo sapiens
RHWGAGVDRLNYEDHCFSGHATMHAENLWPGRLSSVQQILQLSDLWRLTLQKRGCKGLVKVGAPGILQGMVLSFGGLQFTENHLQFQADPDVLHNSYALHGIRYKNDHINLAVLADAEGKPYLHVSVESRGQPVKIYACKAGCLDEPVELTSAPTGHTFSVMVTQPITPLLYISTDLTHLQDLRHTLHLKAILAHDEHMAQQDPGLPFLFWFSVASLITLFHLFLFKLIYNEYCGPGAKPLFRSKEDPSV

SEQ ID NO: 169 - protein, 151 aa, Homo sapiens
PVEADPPEAWLQGAGEGGCPRHPAGDGAQLWGAAVHREPPPVPGRPRRAAQQLCIAWHPLQERPYQPGRAGGCRGQALPTRVRGVPWPACQDLCLQGRLPGRASGADLGAHGPHLLGHGDTAHHATALHLHRPHTPAGPAAHAAPQGHPGP

SEQ ID NO: 170 - protein, 314 aa, Homo sapiens; identical to SEQ ID NO: 66

SEQ ID NO: 171 - protein, 267 aa, Homo sapiens; identical to SEQ ID NO: 166

SEQ ID NO: 172 - protein, 258 aa, Homo sapiens
YDPIGFGLSWEAGRIIGWGKPTRGRGRGGSLSTRGRGSEVPDSAHLAPTPLFSESGCCGLRSRFLTDCKMEEGGNLGGLIKMVHLLVLSGAWGMQMWVTFVSGFLLFRSLPRHTFGLVQSKLFPFYFHISMGCAFINLCILASQHAWAQLTFWEASQLYLLFLSLTLATVNARWLEPRTTAAMWALQTVEKERGLGGEVPGSHQGPDPYRQLREKDPKYSALRQNFFRYHGLSSLCNLGCVLSNGLCLAGLALEIRSL

SEQ ID NO: 173 - protein, 207 aa, Homo sapiens
DGPSTGLVRCLGHANVGDLRLRLPAFPKPSPTYLRTSAEQTLPLLLPHLHGLCLHQPLHLGFTACLGSAHILGGQPALPAVPEPYAGHCQRPLAGTPHHSCHVGPANRGEGARPGWGGTRQPPGSRSLPPAAREGPQVQCSPPEFLPLPWAVLSLQSGLRPEQWALSRWPCPGNKEPLAWALHANKCFFRKKKKKKRGERKRKRREE

SEQ ID NO: 174 - protein, 61 aa, Homo sapiens [sequence omitted]

SEQ ID NO: 175 - protein, 57 aa, Homo sapiens
CLPQYWKCHEFSIRRTLLLVHFKVIPVIVAKESTQWEMEEKCRESKQLVFSFITEVL

SEQ ID NO: 176 - protein, 251 aa, Homo sapiens
AWGPAPAGTALRAGLGVGRAEPHGALRAAPAWASLADFDWVWDDLNKSSATLLSCDNRKVSFHMEYSCGTAAIRGTKELGEGQHFWEIKMTSPVYGTDMMVGIGTSDVDLDKYRHTFCSLLGRDEDSWGLSYTGLLHHKGDKTSFSSRFGQGSIIGVHLDTWHGTLTFFKNRKCIGVAATKLQNKRFYPMVCSTAARSSMKVTRSCASATSLQYLCCHRLRQCGQTRETRWRVCRCRRASSRCYTTSWAGS

SEQ ID NO: 177 - protein, 190 aa, Homo sapiens
GPAAAADPPACLPSLAALAQAVAAQVLEGSGAGTGAGDLHAAPGRRGAHHRVESLVLQLGGCHTYTLPVLEKGECAVPGVQVHANDGALAEPRREAGLVALVVEEARVGEAPAVLIPAQQAAERVAVFVQVHIRRPDAHHHVGAVDGRGHLDLPEVLALPQLLGAPDGRCAAAVLHVEADLTVVTAQQGG

SEQ ID NO: 178 - protein, 178 aa, Homo sapiens
LRKIPHFRSPRHRSHLPAPRWVLAPAPVWQGPGSVGARSRRRRHGVSTSLIRETPFTKAMEVEAAEARSPAPGYKRSGRRYKCLSCTKTFPNAPRGARHAATHGPADCSEEVAEVKPKPETEAKAEEASGEKVSASGAKPRPYRCPLCPKAYKTAPDVRSHRRSHTGEKPFPCPECGR

SEQ ID NO: 179 - protein, 206 aa, Homo sapiens
REVSCKCWTEGGRWDEPHSPLLKNDHLQDSVIPGGQALPALGHTWLRHPWSQCLGWRRPVLAARCGALESSPISFLATPVQRPLQPHSAPTYLGTPAGAREYQGGRAQRDQGKPLAWRVWGTLPQGRTRQEEAQELGPAAQELGPATQAGLGTWQGLGILTLDLGDLGGTGRGEMGGPAPAQCRPHPCTEAPPCHAPRRLPELTGG

SEQ ID NO: 180 - protein, 133 aa, Homo sapiens
ELDGMEDGGQREERKRNAKCGQPPRVKIEGSVMQVLDRRRQVGRAPQPPPQKRPPPGLSDPWGAGSASPRPHVAPAPMVPVPWMETASSGGQMWCSGIQSHFLPGHACPAASSAAFSPYLPGDPGWGTRVPGG

SEQ ID NO: 181 - protein, 138 aa, Homo sapiens
RMKCSQPPRCHFQSDFQKCAPCPRAQTHWLEPPGRVQTISSMRNAQKGFADSIRLWRLPASGVGWVVSPPIQTQEVAPEGMYLVGSSSGTLGGCRALTQVFLSLSSLGCVCACACACLCFSLWAHQDAPRRACARVPT

SEQ ID NO: 182 - protein, 132 aa, Homo sapiens
VHGREARLGTLAGTAALKPALLSGYQTFKGQDVLRRVPVAARRPAGACPRVTAWRCWGSGHLPCLECQEGEAFEEASVLAARSLSQPLPGSCTGQGLIPCHAGPLEQVGWGWYVLSPQPWQPCPLGKVISDL

SEQ ID NO: 183 - protein, 327 aa, Homo sapiens
GQDSPTGKSNILVFKLGGTSLSLSVMEVNSGIYRVLSTNTDDNIGGAHFTETLAQYLASEFQRSFKHDVRGNARAMMKLTNSAEVAKHSLSTLGSANCFLDSLYEGQDFDCNVSRARFELLCSPLFNKCIEAIRGLLDQNGFTADDINKVVLCGGSSRIPKLQQLIKDLFPAVELLNSIPPDEVIPIGAAIEAGILIGKENLLVEDSLMIECSARDILVKGVDESGASRFTVLFPSGTPLPARRQHTLQAPGSISSVCLELYESDGKNSAKEETKFAQVVLQDLDKKENGLRDILAVLTMKRDGSLHVTCTDQETGKCEAISIEIAS

SEQ ID NO: 184 - protein, 416 aa, Homo sapiens
MSSNCTSTTAVAVAPLSASKTKTKKKHFVCQKVKLFRASEPILSVLMWGVNHTINELSNVPVPVMLMPDDFKAYSKIKVDNHLFNKENLPSRFKFKEYCPMVFRNLRERFGIDDQDYQNSVTRSAPINSDSQGRCGTRFLTTYDRRFVIKTVSSEDVAEMHNILKKYHQFIVECHGNTLLPQFLGMYRLTVDGVETYMVVTRNVFSHRLTVHRKYDLKGSTVAREASDKEKAKDLPTFKDNDFLNEGQKLHVGEESKKNFLEKLKRDVEFLAQLKIMDYSLLVGIHDVDRAEQEEMEVEERAEDEECENDGVGGNLLCSYGTPPDSPGNLLSFPRFFGPGEFDPSVDVYAMKSHESSPKKEVYFMAIIDILTPYDTKKKAAHAAKTVKHGAGAEISTVNPEQYSKRFNEFMSNILT

SEQ ID NO: 185 - protein, 208 aa, Homo sapiens
MCGNNMSTPLPAIVPAARKATAAVIFLHGLGDTGPVRPVTLNMNVAMPSWFDIIGLSPDSQEDESGIKQAAENIKALIDQEVKNGIPSNRIILGGFSQGGALSLYTALTTQQKLAGVTALSCWLPLRASFPQGPIGGANRDISILQCHGDCDPLVPLMFGSLTVEKLKTLVNPANVTFKTYEGMMHSSCQQEMMDVKQFIDKLLPPID

SEQ ID NO: 186 - protein, 256 aa, Homo sapiens
LPKYPAPRLLPSLPADLRADSPGGHGRGPHGAQQLLVGARLLGTPSWGPGGHLRLPVCQLAGGAAPRHLPARGQHPAGQLAHCHVQLHIRQSTGQQRSLLEGAGYRLIREFHSRPALAPPFIVISHLRLLLRQLCRRPRSPQPSSPALEHFRVYLSKEAERKLLTWESVHKENFLLARARDKRESDSERLKRTSQKVDLALKQLGHIREYEQRLKVLEREVQQCSRVLGWVAEALSRSALLPPGGPPPPDLPGSKD

SEQ ID NO: 187 - protein, 288 aa, Homo sapiens
AEAGGSLASGGPGPGHASLSQRLRLYLADSWNQCDLVALTCFLLGVGCRLTPGLYHLGRTVLCIDFMVFTVRLLHIFTVNKQLGPKIVIVSKMMKDVFFFLFFLGVWLVAYGVATEGLLRPRDSDFPSILRRVFYRPYLQIFGQIPQEDMDVALMEHSNCSSEPGFWAHPPGAQAGTCVSQYANWLVVLLLVIFLLVANILLVNLLIAMFSYTFGKVQGNSDLYWKAQVTASSGNSTLGPRWPRPLSSSPTCASCSGNCAGDPGAPSRPPRPSSISGFTFLRKPSGSC

SEQ ID NO: 188 - protein, 163 aa, Homo sapiens
  MOD_RES (81): Any amino acid
IYGVSFLIFNIKNIYVSVIPCQGCLLVCLRFCFIFIHVVVIFSSQFLLVSPFPGSFLLLLLSVGDDKLVSLRALHLWIFLXSLTGQPAPVGSGPVLRLPRSLFHLQVCLPXPAPGLAPAAACPSEALLSPPGSHGWFPLSQLVSLNPKPLRNWGLVSGTCCYQ

SEQ ID NO: 189 - protein, 150 aa, Homo sapiens
  MOD_RES (21): Any amino acid
PLSFLMYKTLLSGLEFEHLWXFIYFAXVCGQSNIFPKYILPRKXKKQIRXFDXKXNRPXKGAXTWSRAWXRGKAXRGQVCCGQICAYFITGVKXKQSXIDVXRIYTVXRNXRXXFXKNRNTXWXXFYHXXYTFSLWXNXLTKLXFKIKLM

SEQ ID NO: 190 - protein, 121 aa, Homo sapiens
LVKGMTVLEAVLEIQAITGSRLLSMVPGPARPPGSCWDPTQCTRTWLLSHTPRRRWISGLPRASCRLGEEPPPLPYCDQAYGEELSIRHRETWAWLSRTDTAWPGAPGVKQARILGELLLV

SEQ ID NO: 191 - protein, 117 aa, Homo sapiens
RWHLIRLDQVTRQQQLSRAEAQGRGPAVHLQDPGEPVAVLARSAEIASSVSLQQEQNQLWPRWVGGSAFLAMAAATPRQETAECLEGCNTRSNRQPPLFLMSDGQALQHLDRHGGWS

SEQ ID NO: 192 - protein, 168 aa, Homo sapiens
NTNQFLSITYQYEVKKLRQPFLQIKHMNPMTFLTAEMLPPPQQLGRPVLEGSGCLLGRMCTWNTGCAQITPGTVCAHPVRRKLASPTPWKPRRHTPVLDTCHPEPKRPLTSGAPSTTSAGPCCGQVARTLVPRVPSTVPGTWWILRSACCTEAVCSGKGCSEHRSLCI

SEQ ID NO: 193 - protein, 158 aa, Homo sapiens; identical to SEQ ID NO: 122

SEQ ID NO: 194 - protein, 164 aa, Homo sapiens
AWGRSPGLATRFKDVSDPSRYPLLRGRGRRGSAPGAGPVVGGRAGVVRVVQAALGGSEHALRLHHEHVVEVPHQHRQQWQKRQAVAEASEGAAHGKGQPVDQPGDGKETSEQREQFEGILGQVSGKHGQVEEVENKELDRLGHISPVDLHLPPWLPKGPKGRKA

SEQ ID NO: 195 - protein, 154 aa, Homo sapiens
PVMLFCPSALWGATAGGGDPPAICGPVGPALYSLLLQPGRAFHLPAPGYPQTAPSAHWSLCRLPADLLADLCRGPLLPRLRLRPDVSATAVDADVEPLLHVRGGAGAHAHCHREPPGLPGPRPLGLRLQAPPLGLSLSALALGVGGSGLGLTHL

SEQ ID NO: 196 - protein, 163 aa, Homo sapiens
  MOD_RES (1)..(2): Any amino acid
XXLXSXVXXSWXXSTXATXKXPRWXDKXXSXQARPXXVXTXXQAFSLFQKGSEDGRPASQERPDQXGRGLPQPQVSTRGSIRLSGXPLGSWWDGRGRWKRAEEMGSGWRVCASFEATSAGHRLLNTKLVPTDELAMEEIXGRPLXPTGRAAHNSTSFPDRAGF

SEQ ID NO: 197 - protein, 152 aa, Homo sapiens
  MOD_RES (36): Any amino acid
ATCGLHLWPRRKHKPSIHFPFPLPFSTSPFHPTSCXVAXQKALLSPLLTLGAAXGLSXTGLAFPERQVFRPQSLSGTRRMPVXRWXHXXAWPVRXSTCLXSGXAXTLPEWXEXMTXPLXXPRXXREVSGYAGFPSSSWXAEFLGXXQIFXTL

SEQ ID NO: 198 - protein, 143 aa, Homo sapiens
SCGPRGIASLGLGFSGRCDDQNKGRSRRARGSGGGVFRGAHLPGAAGQPEPHRAALASRRLTRKLYKCIKKAVKQKQIRRGVKEVQKFVNKGEKGIMVLAGDTLPIEVYCHLPVMCEDRNLPYVYIPSKTDLGAAAGPSAPPV

SEQ ID NO: 199 - protein, 228 aa, Homo sapiens
MASEELQKDLEEVKVLLEKATRKRVRDALTAEKSKIETEIKNKMQQKSQKKAELLDNEKPAAVVAPITTGYTVKISNYGWDQSDKFVKIYITLTGVHQVPTENVQVHFTERSFDLLVKNLNGKSYSMIVNNLLKPISVEGSSKKVKTDTVLILCRKKVENTRWDYLTQVEKECKEKEKPSYDTETDPSEGLMNVLKKIYEDGDDDMKRTINKAWVESREKQAKGDTEF

SEQ ID NO: 200 - DNA, 13 bp, Artificial Sequence
  Description of Artificial Sequence Partial cDNA sequence e.g., EST or contig S
gcctcaagttatc

SEQ ID NO: 201 - DNA, 29 bp, Artificial Sequence
  Description of Artificial Sequence Consensus sequence C
atgtcctagcctcaagttatcagatgcaa